(19) United States
(12) Patent Application Publication
(10) Pub. No.: US 2023/0304024 A1
(43) Pub. Date: Sep. 28, 2023

(54) Modulation of Gene Expression Via Transcription Factor-Chemically Induced Proximity (TF-CIP)

(71) Applicant: The Board of Trustees of the Leland Stanford Junior University, Stanford, CA (US)

(72) Inventors: Gerald R. Crabtree, Stanford, CA (US); Sai Gourisankar, Stanford, CA (US); Andrey Krokhotin, Stanford, CA (US); Chiung-Ying Chang, Stanford, CA (US); Wendy Christine Wenderski, Stanford, CA (US); Samuel Heeju Kim, Stanford, CA (US); Nathanael Gray, Stanford, CA (US); Xiaofan Liu, Stanford, CA (US); Zhengnian Li, Stanford, CA (US); Tinghu Zhang, Stanford, CA (US)

(21) Appl. No.: 18/143,492

(22) Filed: May 4, 2023

Related U.S. Application Data
(63) Continuation of application No. PCT/US2021/058231, filed on Nov. 5, 2021.
(60) Provisional application No. 63/110,575, filed on Nov. 6, 2020.

(51) Int. Cl. C12N 15/67; A61K 47/55; C07K 14/47; A61P 35/00

(52) U.S. Cl. CPC C12N 15/67; A61K 47/55; C07K 14/4702; A61P 35/00

(57) ABSTRACT

Methods of modulating transcription of a target gene in a cell (which may be in vitro or in vivo) are provided. Aspects of the methods employ a transcription factor-chemical inducer of proximity (TF-CIP) system to modulate, e.g., enhance or reduce, transcription of a target gene in a cell. Embodiments of the methods include providing in a cell a chemical inducer of proximity (CIP) which links a first endogenous anchor transcription factor that binds to a promoter of the target gene and a second endogenous transcription modulating factor (e.g., a transcription factor or transcription repressor), wherein CIP mediated linkage of the anchor transcription factor and transcription modulating factor modulates transcription of the target gene in the cell. Also provided are compositions that find use in practicing methods of the invention.

### CROSS-REFERENCE TO RELATED APPLICATIONS

This application is a continuation application of PCT Application Serial No. PCT/US2021/058231 filed on Nov. 5, 2021; which application claims priority to the filing date of U.S. Provisional Patent Application Ser. No. 63/110,575, filed Nov. 6, 2020; the disclosures of which applications are incorporated herein by reference.

### GOVERNMENT RIGHTS

This invention was made with Government support under contract CA163915 awarded by the National Institutes of Health. The Government has certain rights in the invention.

### REFERENCE TO AN ELECTRONIC SEQUENCE LISTING

The contents of the electronic sequence listing (STAN-1783_SEQ_LIST.xml; Size: 15,606 bytes; and Date of Creation: Jun. 1, 2023) is herein incorporated by reference in its entirety.

### INTRODUCTION

Methods of controlled regulation of gene expression have been increasingly important in a wide range of areas, including, but not limited, to gene therapy, synthetic biology, plant management, environmental clean-up, bacterial and microbial management and synthetic genetic circuits. Control of gene expression holds vast potential at revolutionizing therapeutics, animal models, and biotechnological processes and is useful to integrate multiple input signals for cell-based therapy and animal model development. Despite rapid advances in recent years, precise control of gene expression remains a challenge due to unpredictability stemming from unintended interactions between biological components, such as transcription factors, etc. A fundamental goal in cellular engineering is to predictably and efficiently express genes at a desired level and under precise control. Such genetically engineered cells hold great promise for advancing therapeutics, diagnostics, animal models, and biotechnological processes.

To date, a variety of different gene modulation technologies for modulating gene expression in a cell have been developed. Such gene modulation technologies include RNA interference, DNA editing and expression, and chemical compounds that suppress, enhance, or modify gene expression. These can be in the form of RNA, DNA, or protein, and can be introduced into cells in culture through direct application to media, lipofection, electroporation, or viral transduction.

However, because of the wide applicability of gene modulation to both research and therapeutic applications, there is a continued interest in the development of new ways to modulate transcription of a target gene in a cell, specifically to modulate expression of genes without genetic modification.

### SUMMARY

Methods of modulating transcription of a target gene in a cell, without genetic modification, where the methods may be in vitro or in vivo, are provided. Aspects of the methods employ a transcription factor-chemical inducer of proximity (TF-CIP) system to modulate, e.g., enhance or reduce, transcription of a target gene in a cell. Embodiments of the methods include providing in a cell a chemical inducer of proximity (CIP) which links a first endogenous anchor transcription factor that binds to a promoter of the target gene and a second endogenous transcription modulating factor (e.g., a transcription factor or transcription repressor), wherein CIP mediated linkage of the anchor transcription factor and transcription modulating factor modulates transcription of the target gene in the cell without a need for genetic modification. Also provided are compositions that find use in practicing methods of the invention.

### BRIEF DESCRIPTION OF THE DRAWINGS

FIG. 1 provides a general overview of TF-CIP mediated modulation of transcription. An anchor transcription factor binding ligand A recruits or hijacks by chemically induced proximity a second transcription factor that binds ligand B to activate or repress transcription of a target gene or genes to produce a therapeutic effect.

FIG. 2 illustrates general chemical structures of embodiments of TF-CIPs. As illustrated, a TF-CIP according to an embodiment includes a moiety that binds one transcription regulator linked by a chemical linker to a second moiety that binds to an anchoring transcription factor that provides genomic localization. This structure includes linkers of different length and composition. As illustrated, TF-CIPs may also be configured as “molecular glues” in which the A and B moieties are incorporated into a molecular glue that also uses the interactions between the two proteins to aid the interaction.

FIG. 3 provides a description of building a TF-CIP by rational design. FIG. 3 illustrates the design of a TF-CIP to hijack BCL6 to kill ER-positive breast cancer cells or AR-positive prostatic cancer cells. BCL6 is a transcription factor and oncogene that prevents death of a variety of cancer cells including breast cancer cells by binding epigenetic repressors, BOOR, NCOR and SMRT (PMID 18280243, 15531890, 10898795). Several inhibitors of BCL6's repressive function have been produced by other groups that prevent the binding of BOOR, NCOR and SMRT to the site formed by the dimeric surface of BCL6 (PMID15531890), however these inhibitors have not been sufficiently active to be used therapeutically (PMID30335946). Chemical linkage of BCL6 inhibitors, such as B13812(PMID33208943) to estrogen compounds that then bind and induce proximity to the cell death (proapoptotic) promoters, such as those for TP53, PUMA, BIM and others, convert the inhibitor of cell death to a powerful activator of cell death.

FIG. 4 provides chemical structures of TF-CIPs and their components designed to hijack BCL6's repressive activity and convert it to an activator of cell death, e.g., as illustrated in FIG. 3. Each structure illustrated in FIG. 4, except compounds 1 and 4, which are linker controls) includes an estrogen receptor binding moiety connected by a chemical linker to a BCL6 inhibitor based on the previously described molecule B13812 (PMID33208943). Also shown is a structure using a similar strategy employing an androgen analogue linked to a BCL6 inhibitor based on the previously described molecule B13812(PMID33208943). Also shown is a structure containing the BCL6 inhibitor and a linker to control for linker effects.

FIGS. 5A to 5C illustrate the development of reporters for activation of cell death or pro-apoptotic pathways by the disinhibited BCL6 protein or the pro-apoptotic protein FOXO3A. FIG. 5A: The BCL6 reporter consists of an array of BCL6 binding sites taken from different pro-apoptotic genes including TP53, PUMA, BIM and others. These genes have the ability to kill cells when simply overexpressed (PMID:11463391). Ten base pairs on either side of the actual human BCL6 binding site are included to take advantage of the fact that transcriptional specificity is due to the concerted binding of proteins in an enhanceosome (PMID:9510247, 18206362).

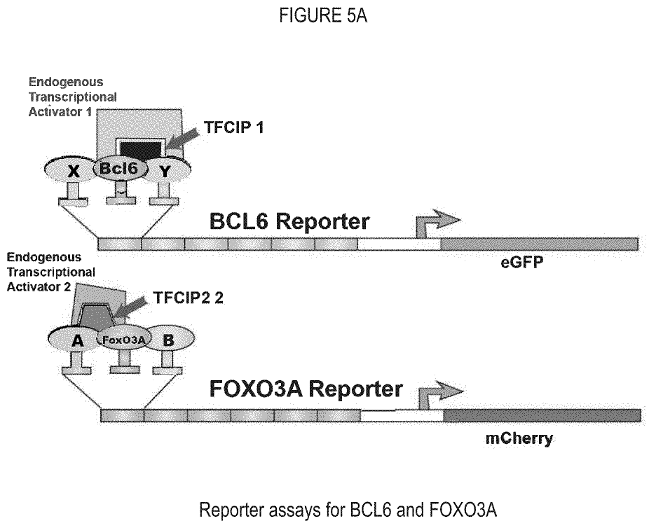

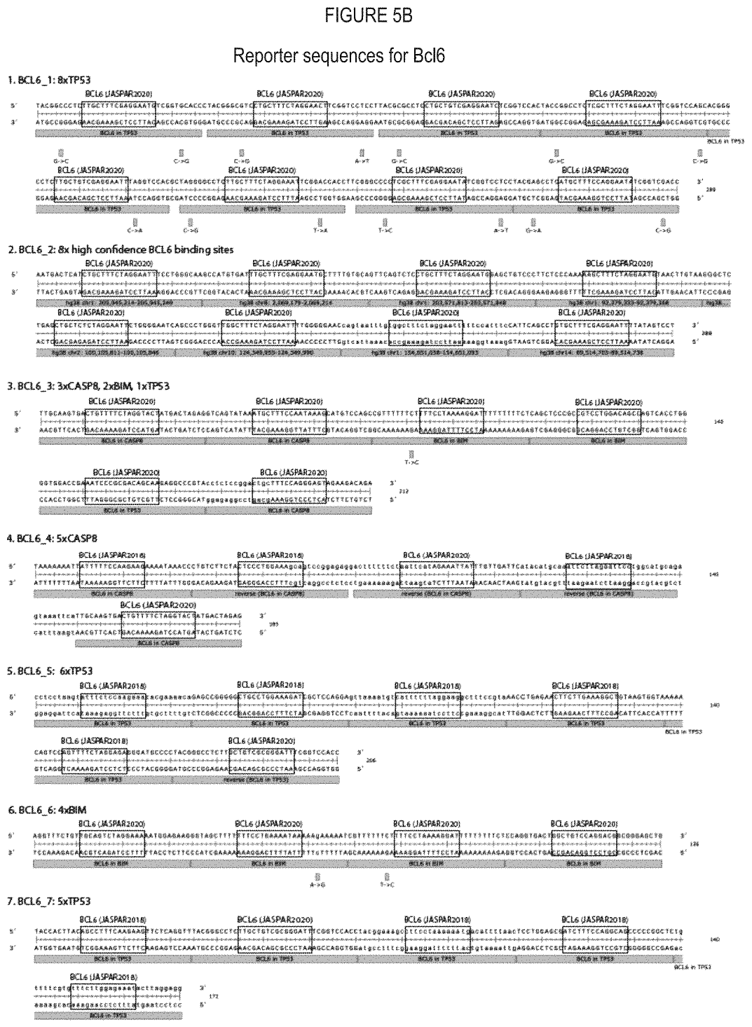

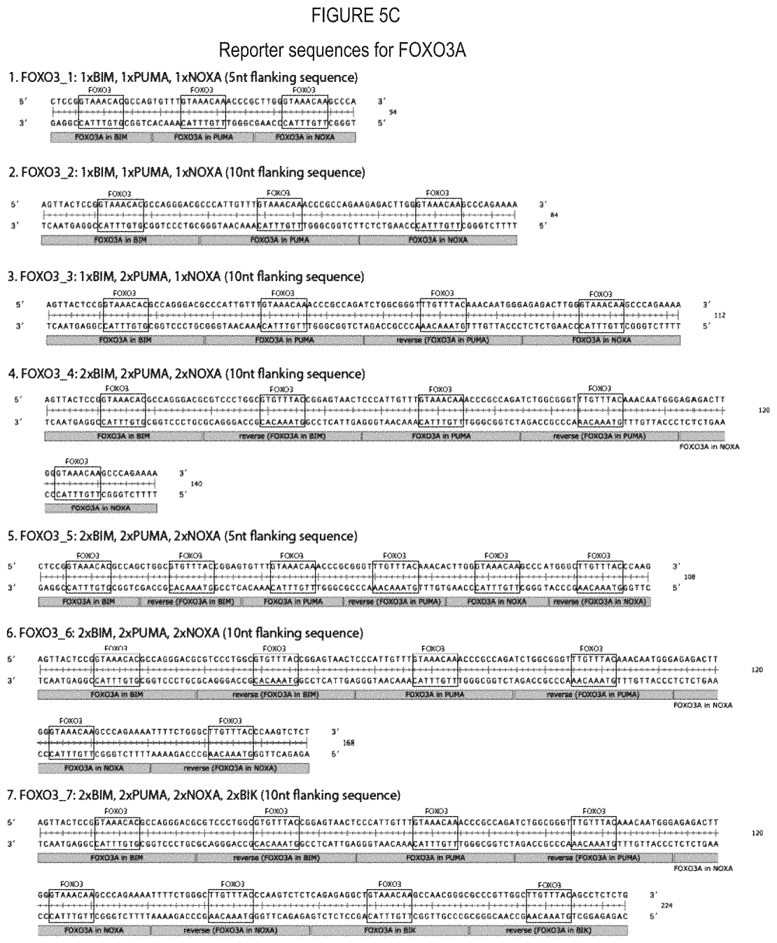

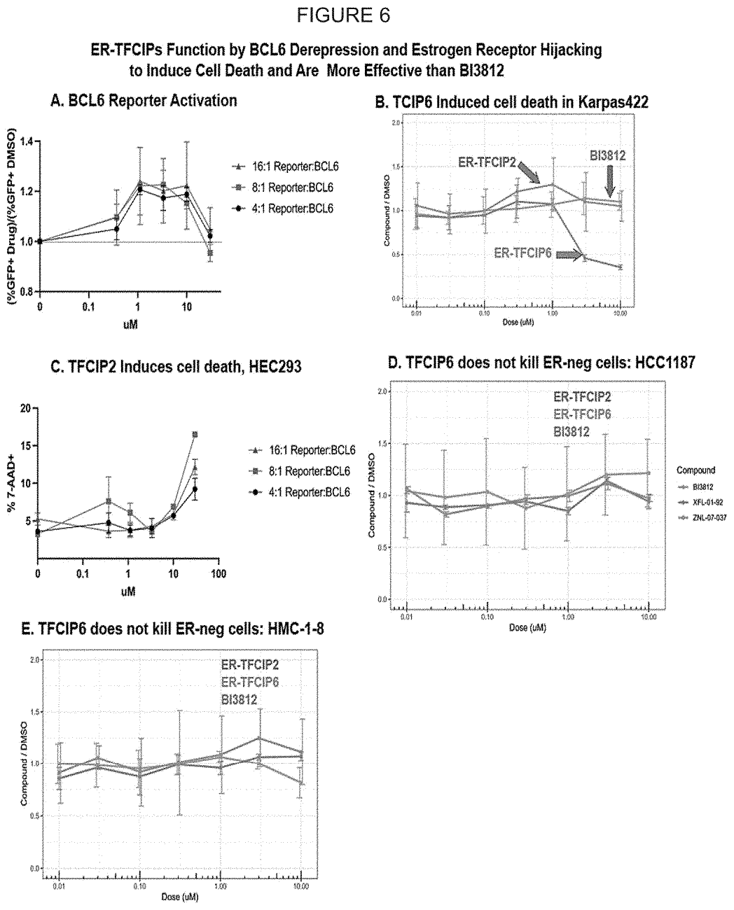

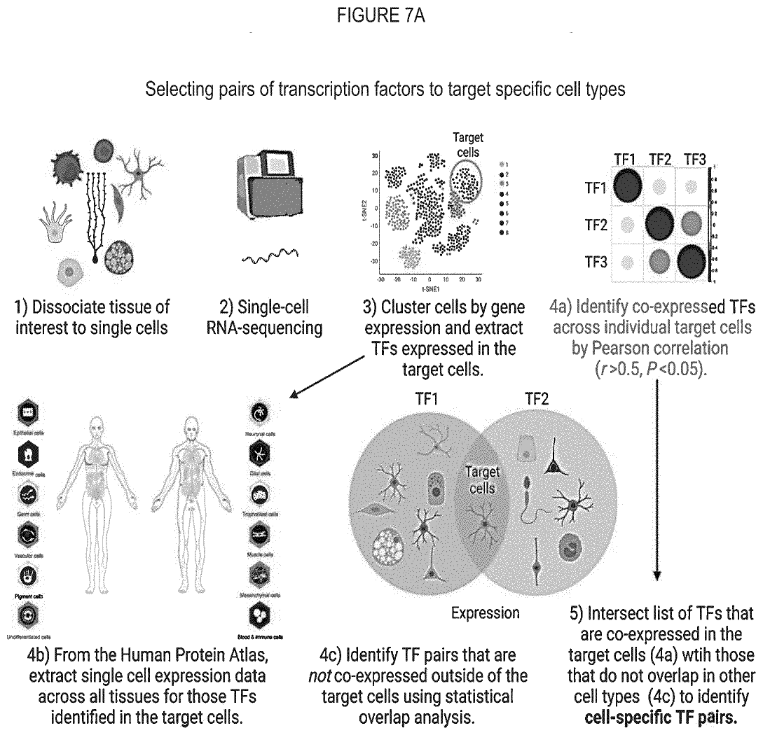

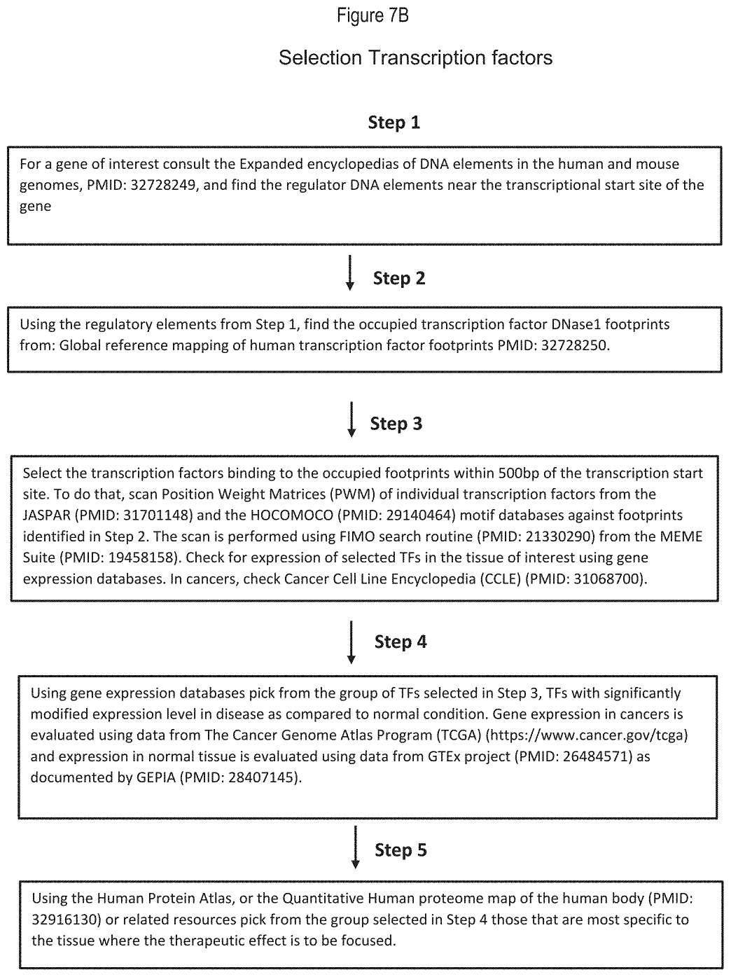

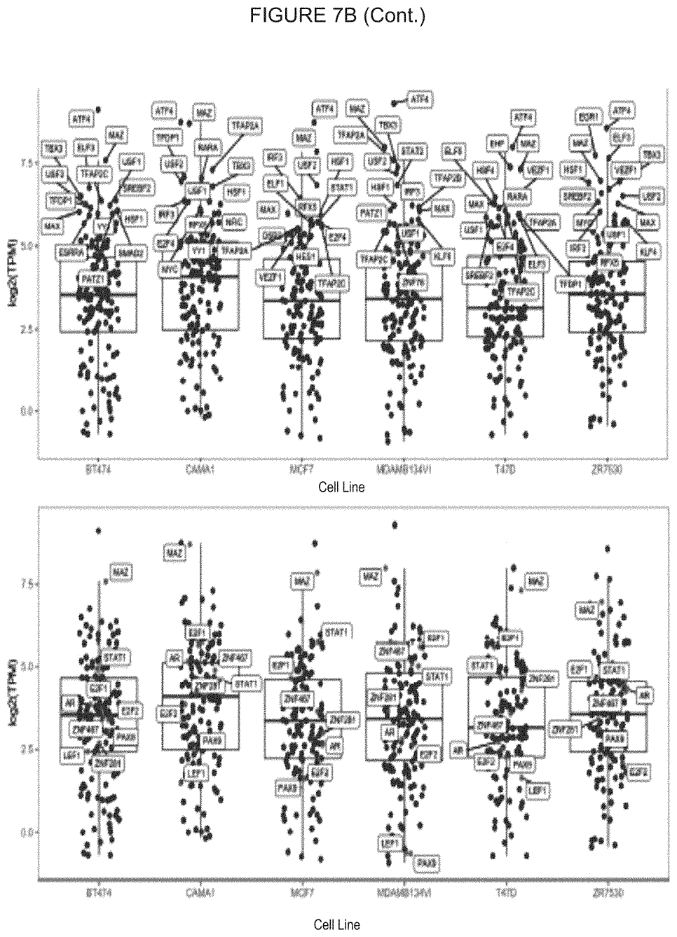

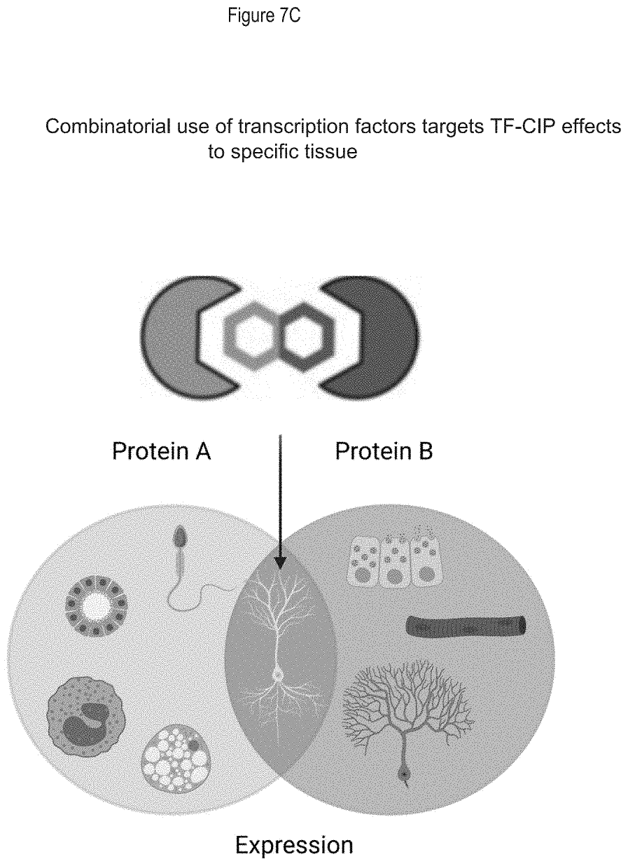

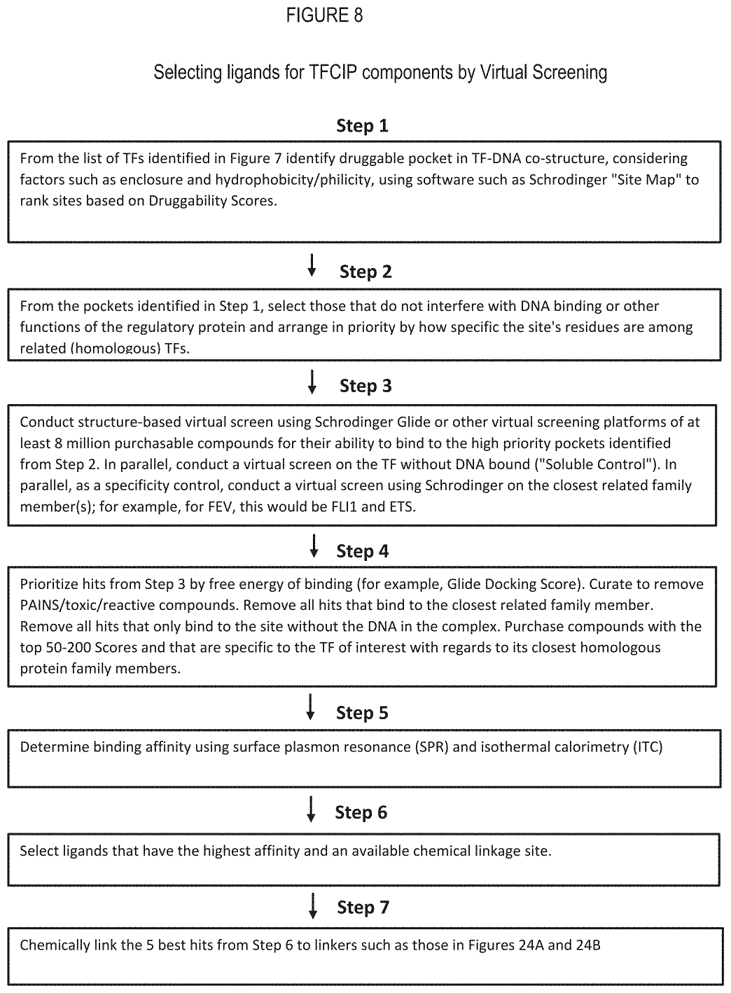

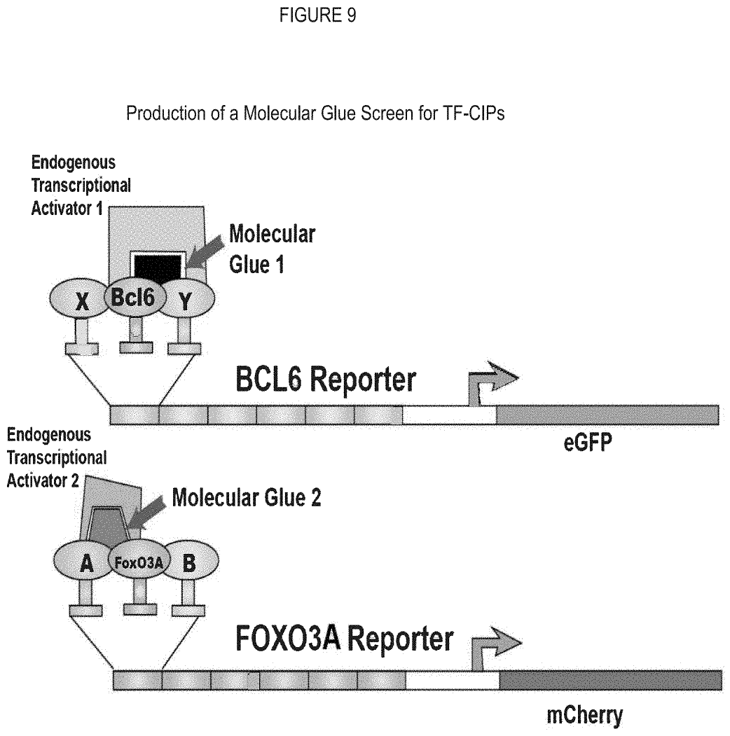

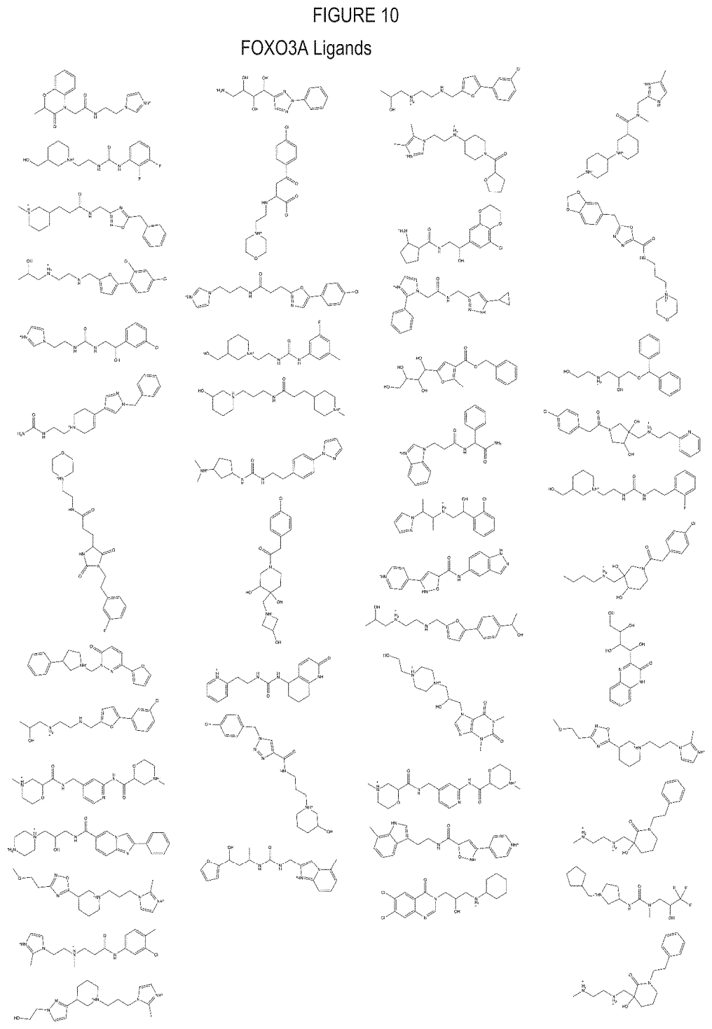

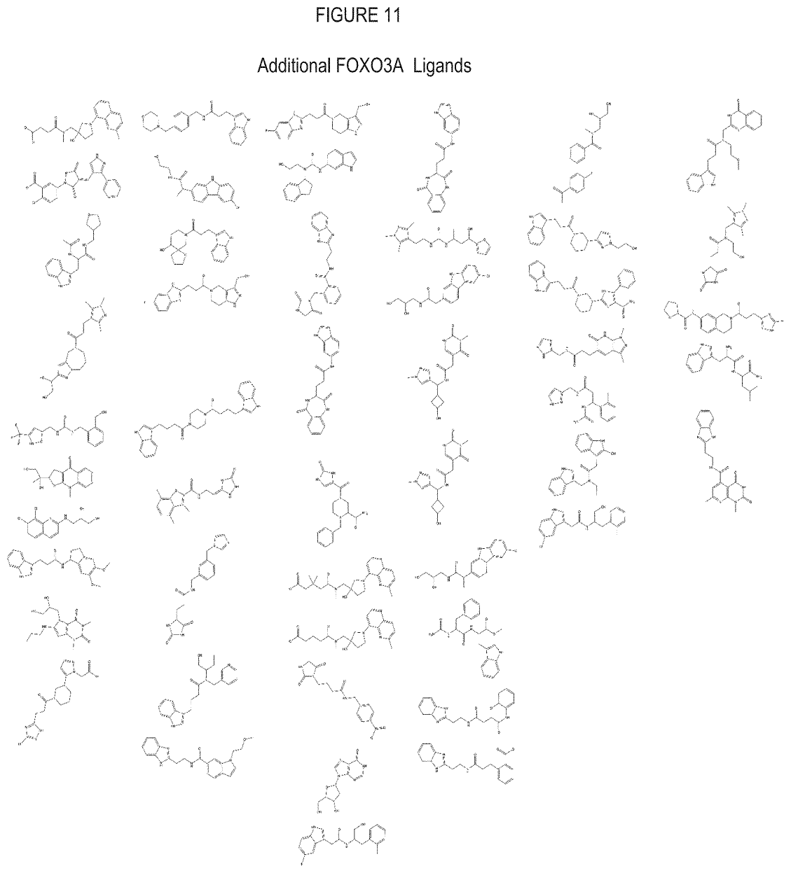

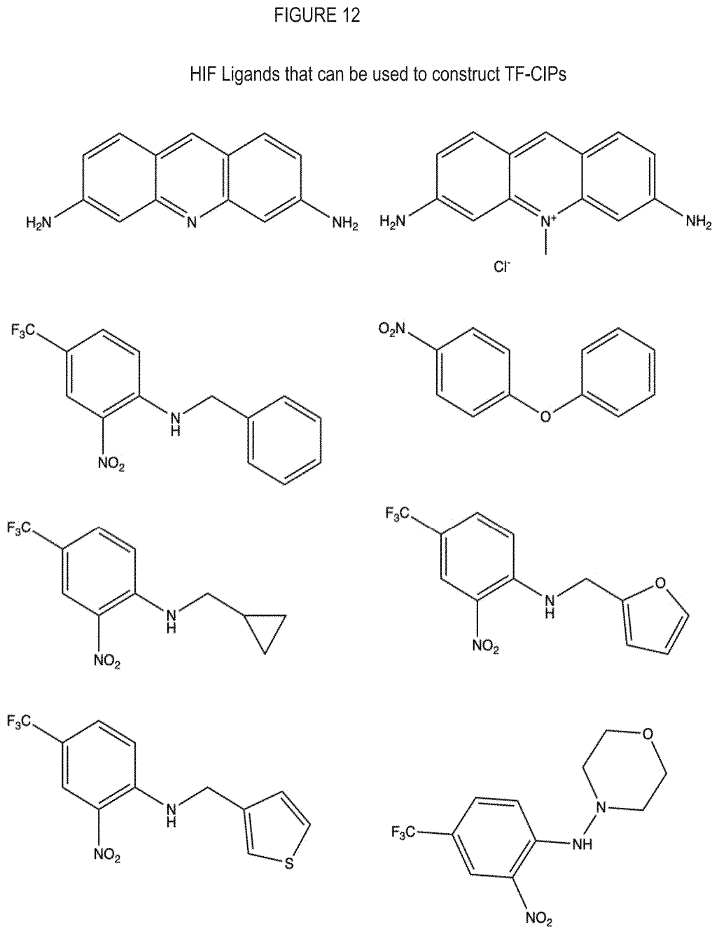

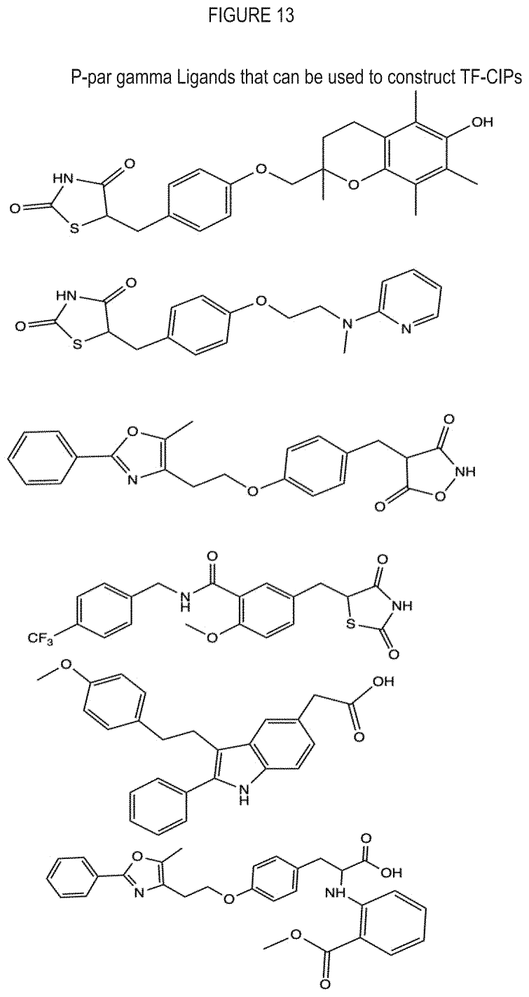

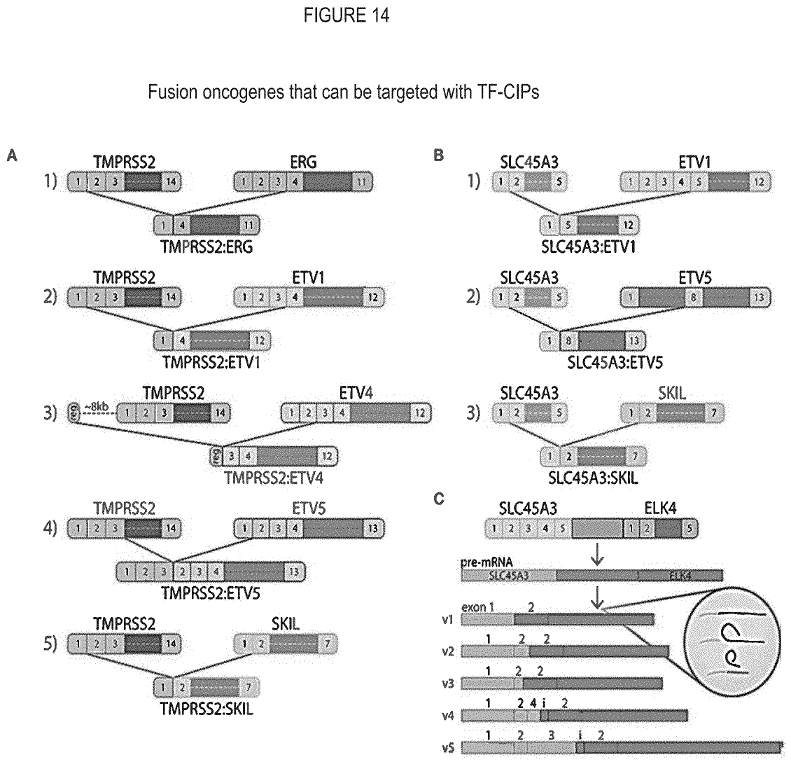

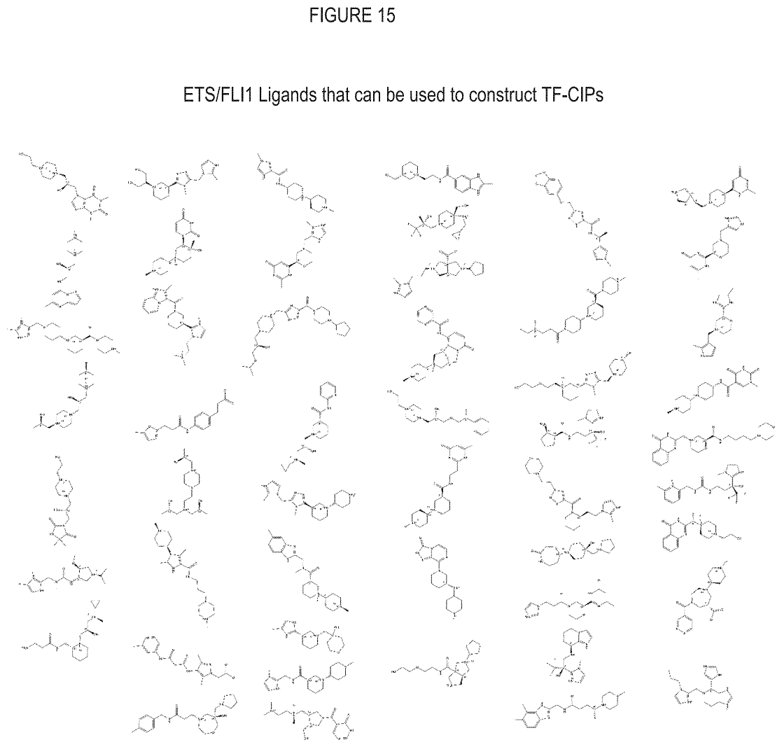

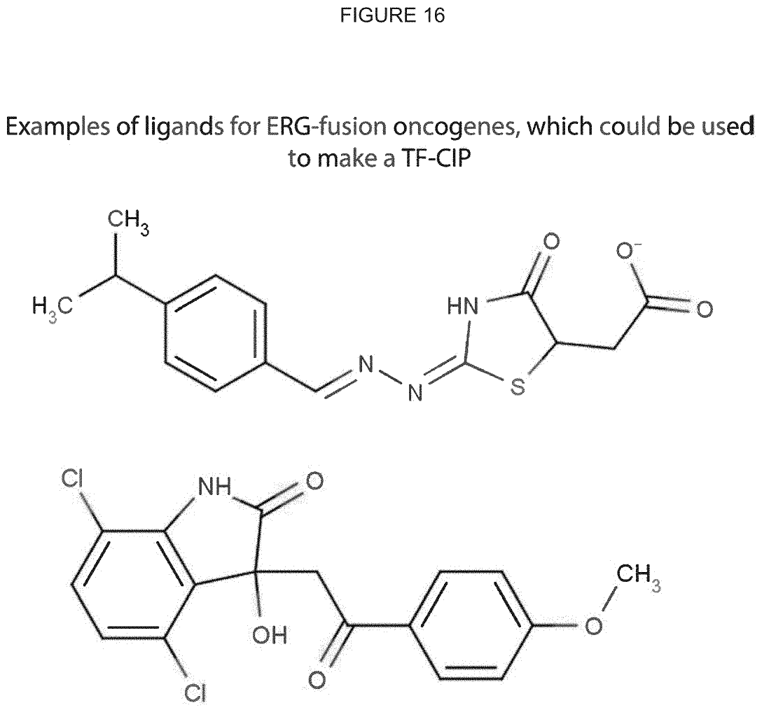

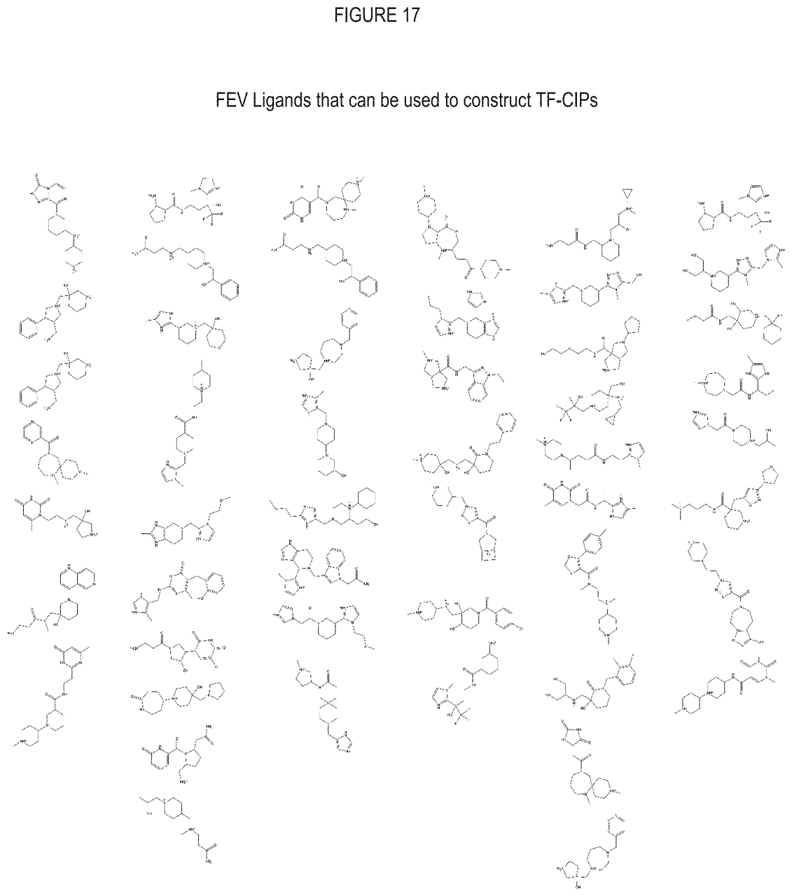

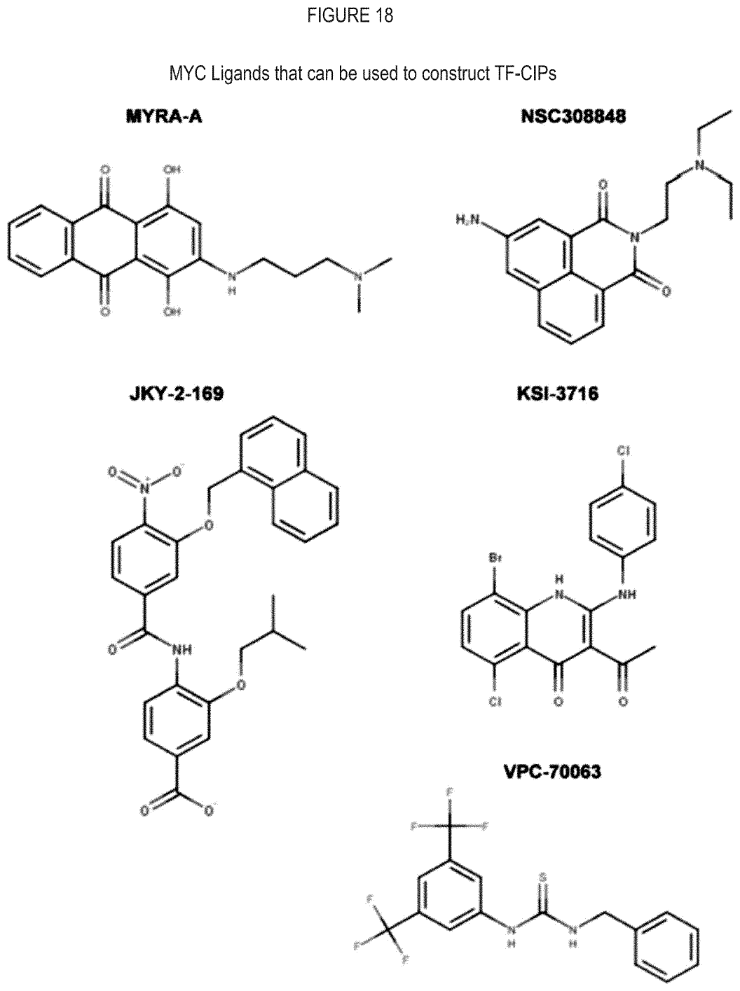

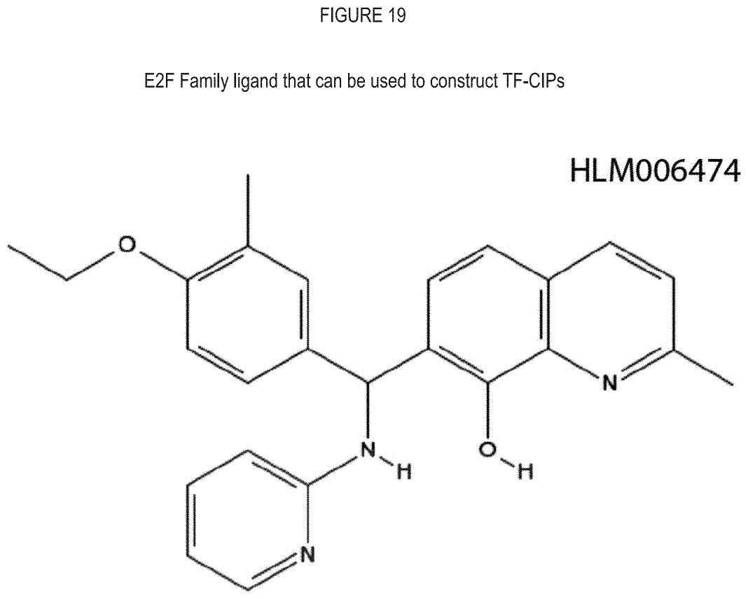

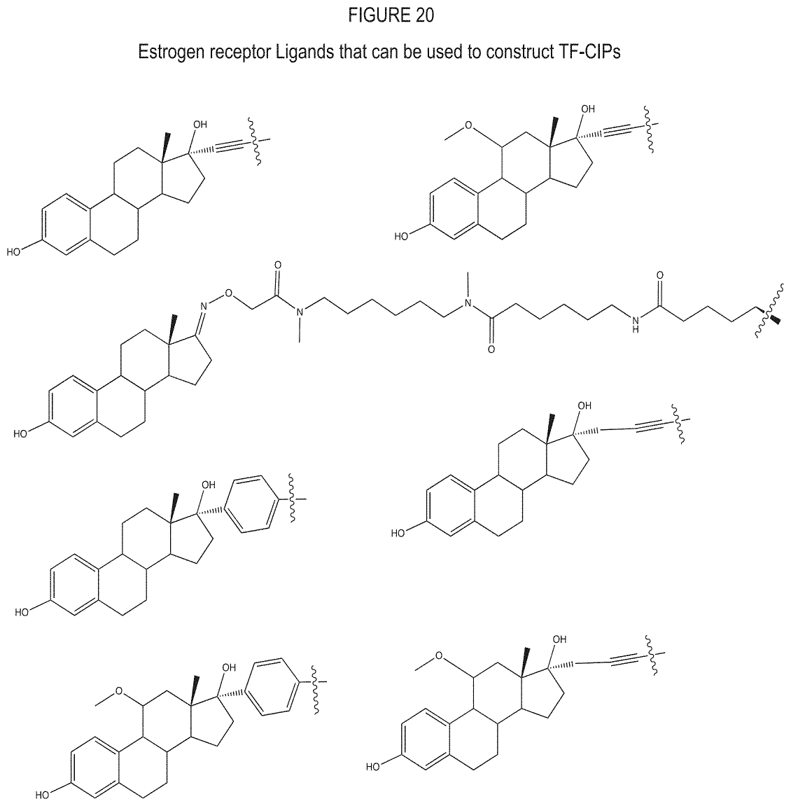

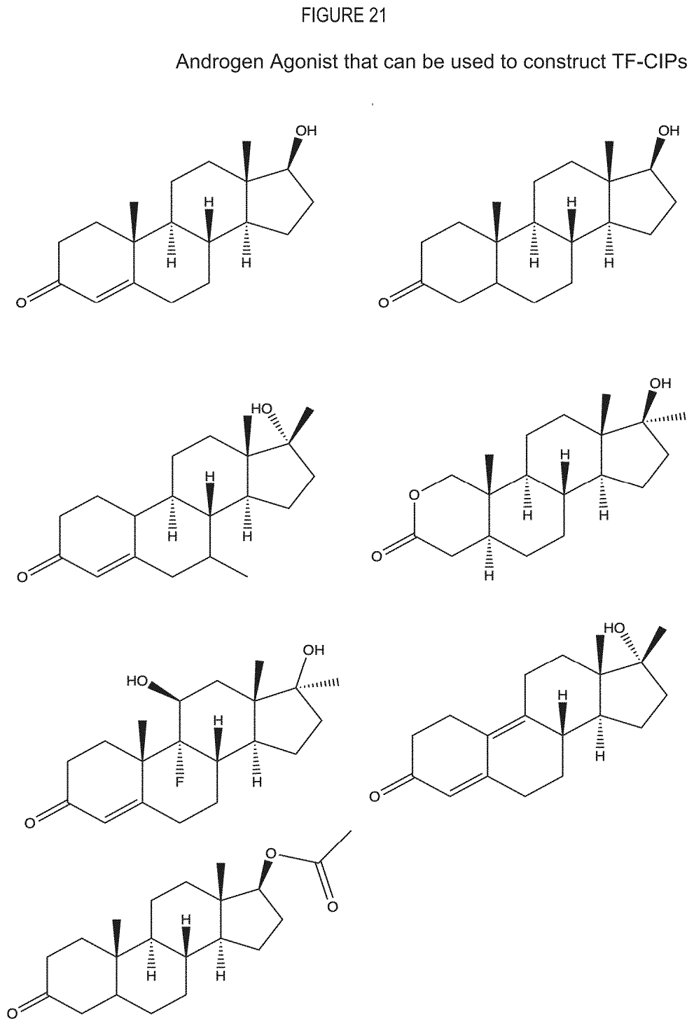

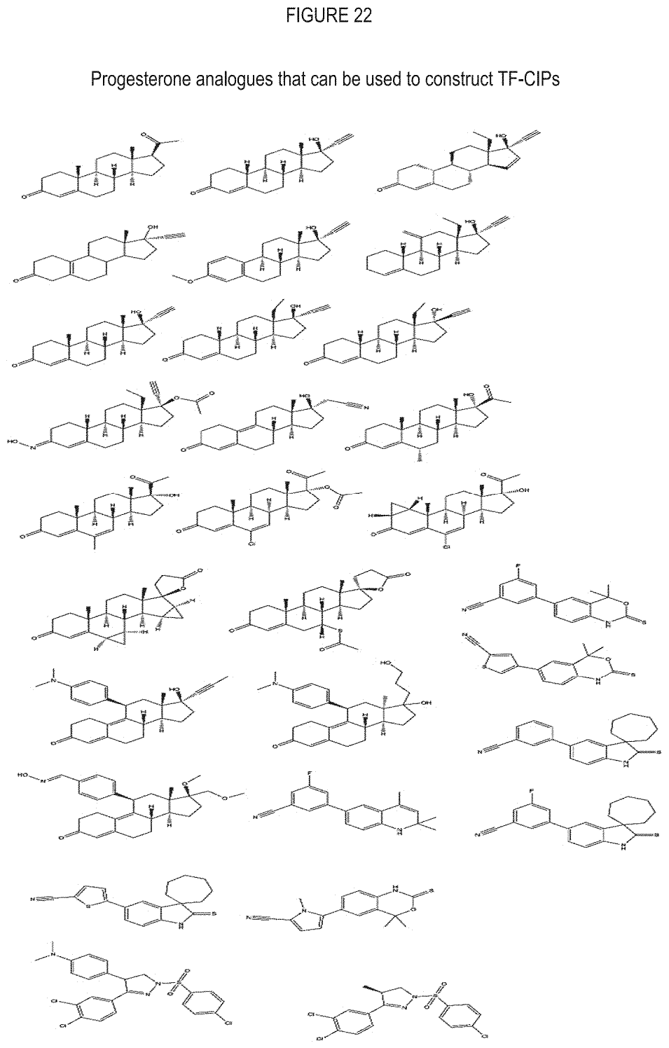

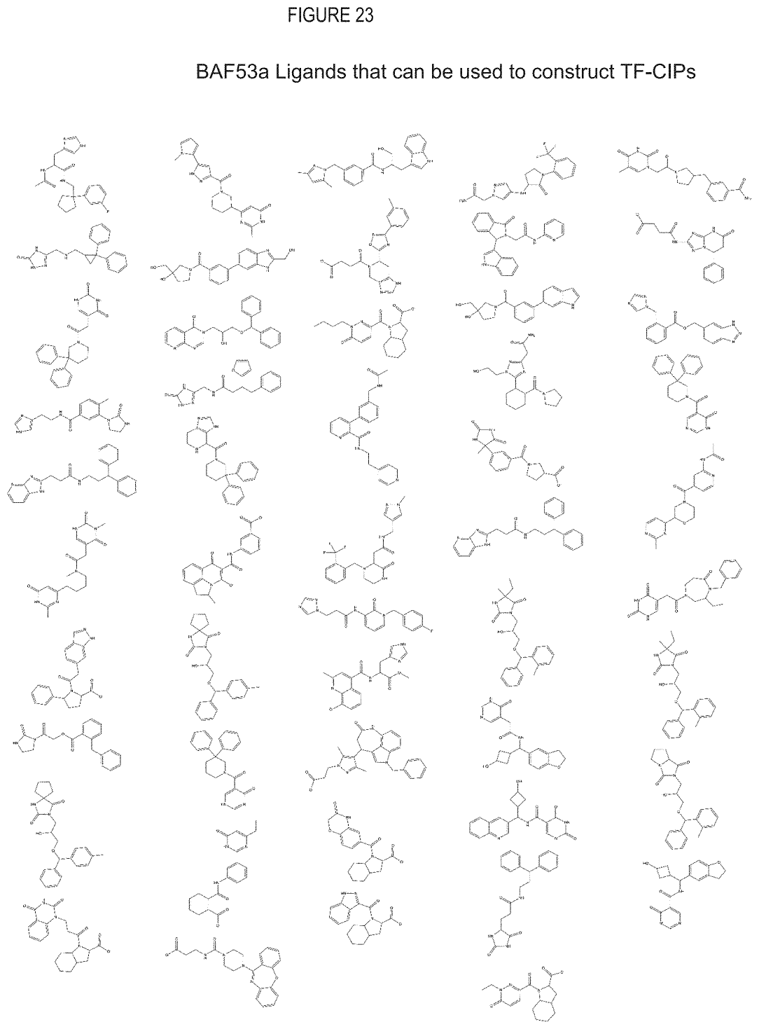

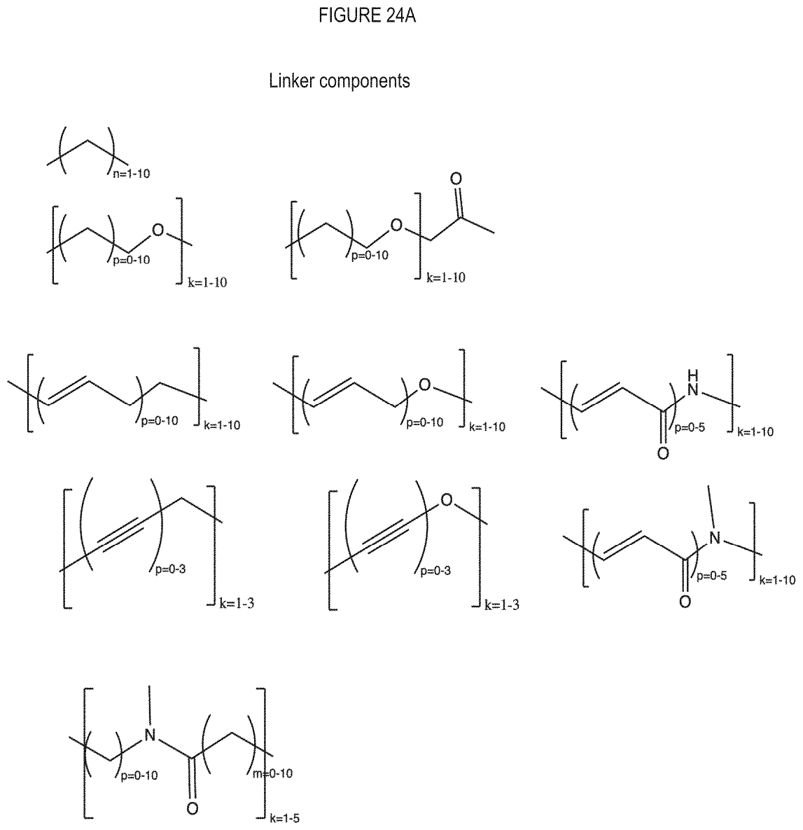

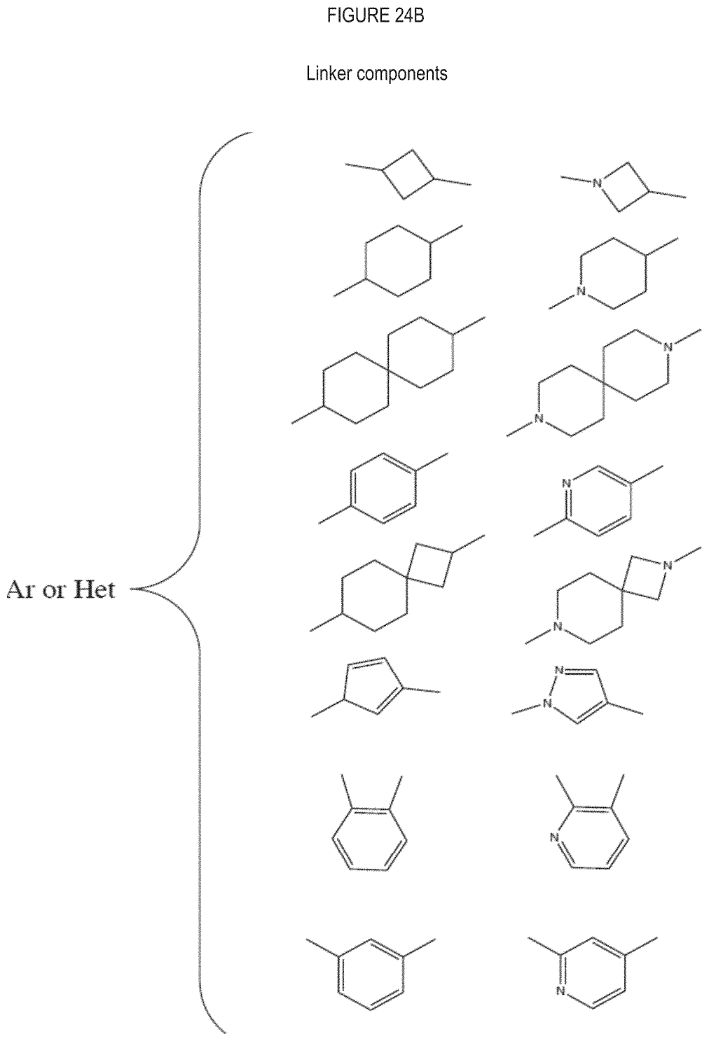

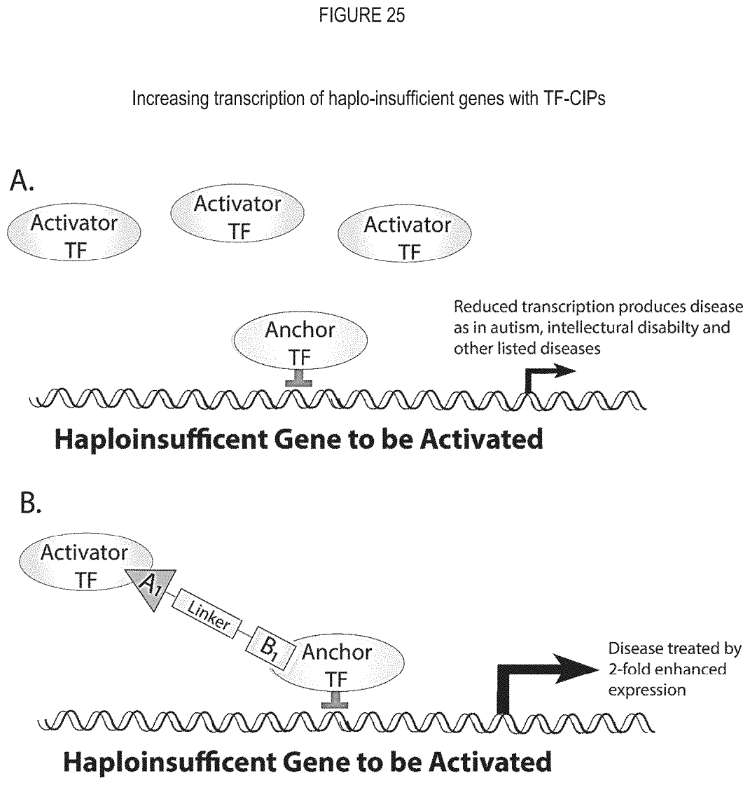

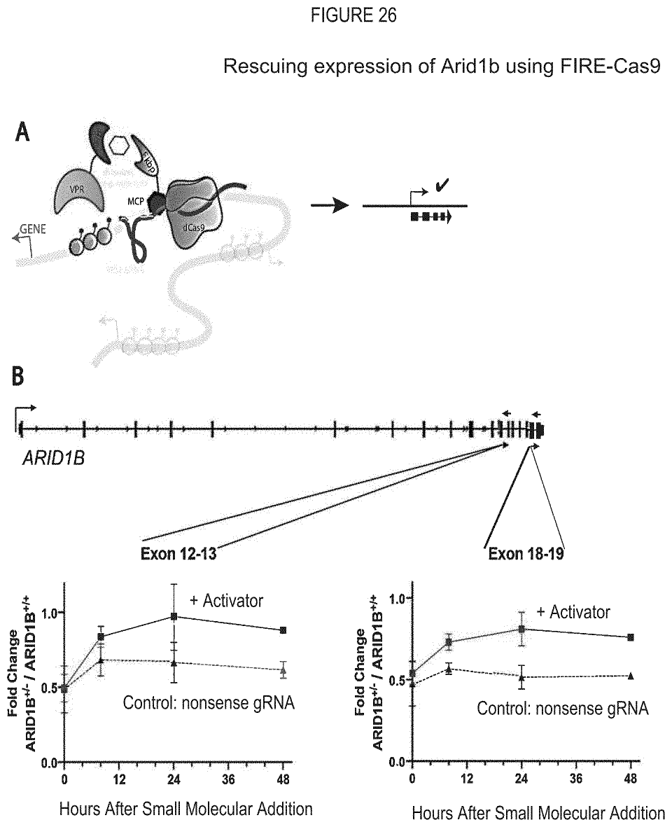

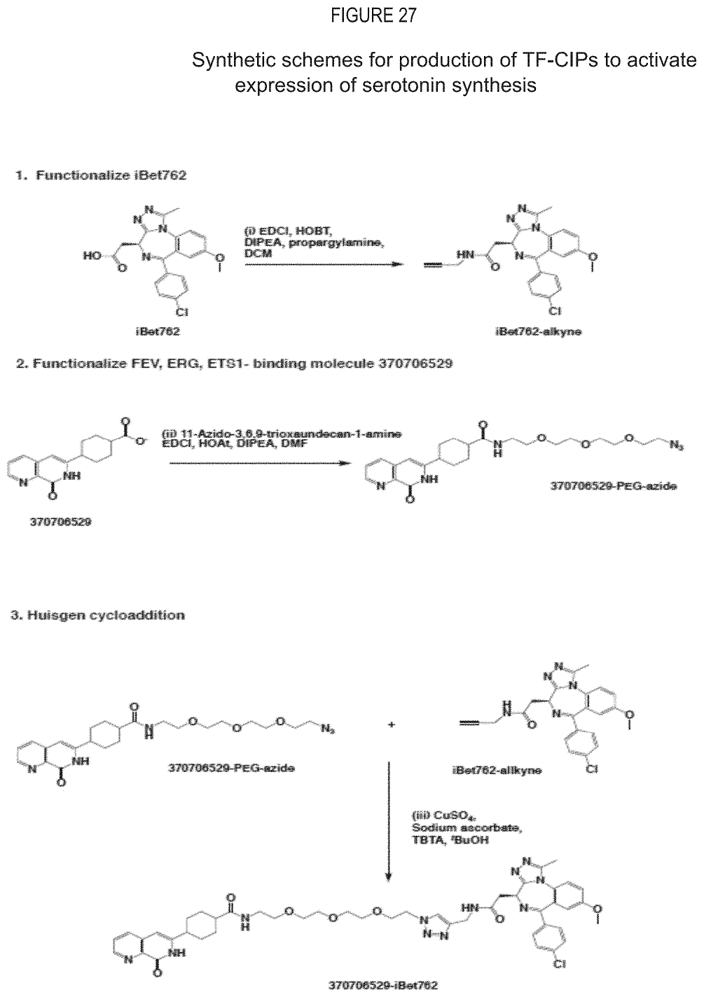

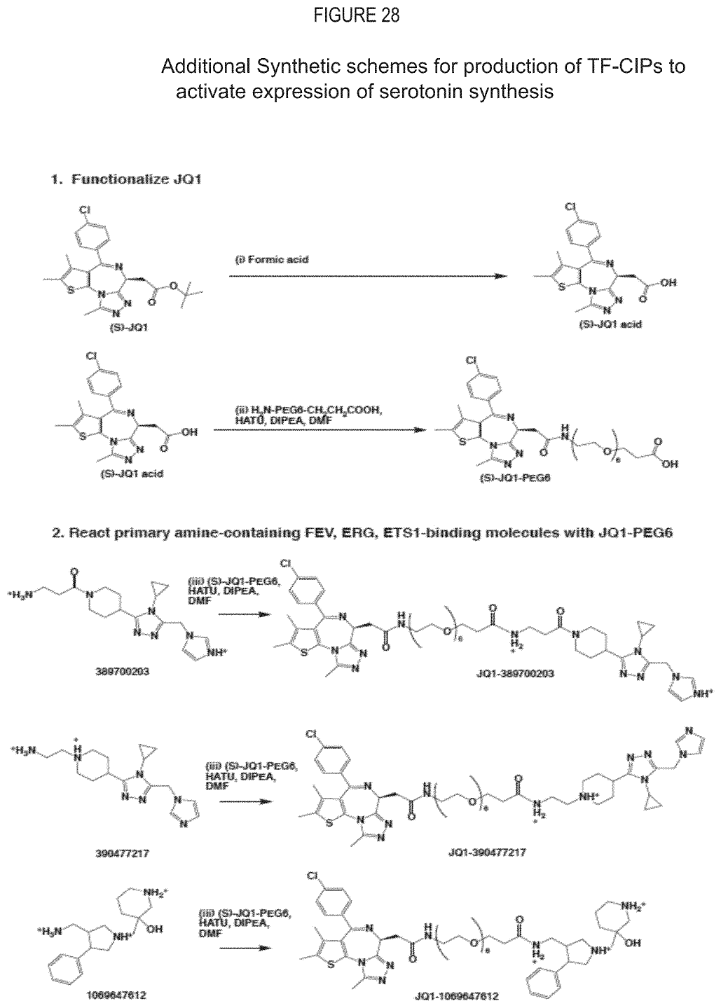

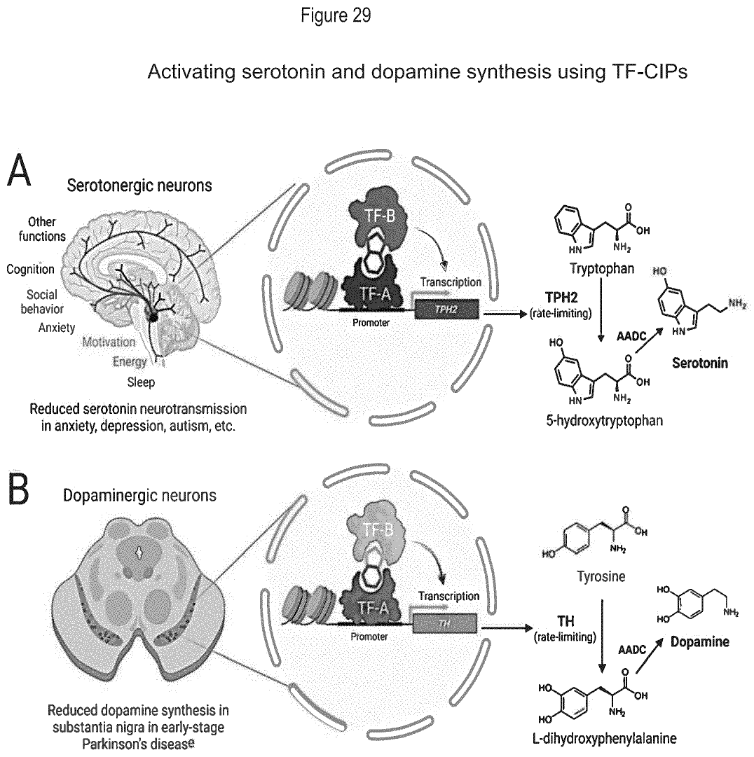

Thus, the reporter system is multiplexed and useful for defining apoptotic responses in many different cell types. The FOXO3A reporter consists of an array of FOXO3A binding sites taken from different pro-apoptotic genes including TP53, PUMA, BIM and others. These genes have the ability to kill cells when simply overexpressed. Ten base pairs on either side of the actual human FOXO3A binding site are included to take advantage of the fact that transcriptional specificity is due to the concerted binding of proteins in an enhanceosome (PMID9510247, 18206362). Thus, the reporter system is multiplexed and useful for defining apoptotic responses in many different cell types. FIG. 5B provides DNA sequences of the reporters for disinhibited BCL6. FIG. 5C provides DNA sequences for the reporter for the activation of FOXO3A

FIG. 6: ER-TF-CIPs act by BCL6 De-repression to Activate BCL6 Reporters and Induce cell death more effectively than BI3812. Panel A: Addition of ER-TF-CIP2 activates GFP expression by the BCL6 reporter at about 1 micromolar indicating that it is more potent than BI3812. Note that the higher concentrations lead to a reduction in activation of the reporter consistent with the “hook effect” characteristic of bifunctional molecules as they saturate both binding sites.(PMID: 8752278; PMID:21406691), in this case the BCL6 protein and the estrogen receptor. Panel B. ER-TFCIP6 induces cell death of ER-positive cells with amplified BCL6 (Karpas 442) more effectively than BI3812. Panel C. ER-TFCIP6 induces cell death in HEC293 cells. Panels D and E demonstrate that ER-TF-CIP6 does not kill two different ER-negative breast cancer cell lines, hence cell death is dependent upon high levels of estrogen receptor expression as occurs in breast cancer. Viability was measured using the PrestoBlue HS (resazurin) assay for viable cells (1:10 ratio in media and 1 h incubation).

FIGS. 7A to 7C provide a step-by-step illustrations of how to pick transcription factor pairs for a specific target gene expressed in a specific cell type of interest. FIG. 7A illustrates a general method of selecting cell-type specific transcription factor pairs that can be used to define and anchor transcription factor and a activating transcription factor each with selective expression in the tissue of interest. FIG. 7B: the anchoring transcription factors for treatment of breast cancer are illustrated that bind to the promoters of specific target genes. FIG. 7C provides an illustration of the combinatorial use of transcription factors targets TF-CIP effects to specific tissue.

FIG. 8 provides a protocol for selecting a ligand to be used in a TF-CIP for a transcription factor, e.g., as identified using the protocols illustrated in FIGS. 7A to 7C. Selection of a pocket within the anchoring TF involves the use of Site Map (PMID: 19434839) for example, but other programs can also be used. Docking to the pocket and scoring of hits are described in PMID: 17034125 and PMID: 15027866. Other methods of selecting ligand include DNA-encoded library screening (PMID 28094476) with the protein of interest. Yet another way of selecting ligands is by screening libraries of small molecules using fluorescence polarization or other direct methods of measuring binding.

FIG. 9: Screen for TFCIPs that act as molecular glues using the reporters introduced in FIG. 5A. A “molecular glue” (PMID 33417864) is distinguished from a “bifunctional molecule” such as those illustrated in FIG. 2, by virtue of the fact that the linker has been replaced with a more direct connection between the two binding moieties. Molecular glues, such as FK506, often have good pharmacologic behavior. Most importantly, they can engage several proteins at once (PMID 33417864) by promoting interactions between the target protein, such as a cancer driver, and several proteins within a highly biologically specific enhanceosome (PMID:9510247, 18206362). The reporters for the screen are shown in FIGS. 6A and B.

FIG. 10 provides the structures for ligands for FOXO3A that bind to a proximal pocket adjacent to the DNA. The general method for discovery of these ligands is given in FIG. 8.

FIG. 11 provides the structures for ligands for FOXO3A that bind a site distal to the DNA, on the “back” of the DNA binding domain such that they do not interfere with DNA binding. The general method for discovery of these ligands is given in FIG. 8.

FIG. 12 provides the structures for known ligands for HIF1a that could be used for the synthesis of TF-CIPs that bring a cancer driver to the promoters of pro-apoptotic genes which are involved in cell death. These ligands are described in: PMID: 19950993 PMCID: PMC2819816 DOI: 10.1021/ja9073062PMID: 19129502 PMCID: PMC2626723 DOI: 10.1073/pnas.0808092106.

FIG. 13 provides the structures for ligands for ppar-gamma, which binds to the promoters of proapoptotic genes and could be used to synthesize a TF-CIP that brings a cancer driver to the promoters of proapoptotic genes which are involved in cell death. These ligands are described in: PMID: 24272485 DOI: 10.1158/0008-5472.CAN-13-1836PMID: 11900961 DOI: 10.1016/s0739-7240(01)00117-5

FIG. 14 provides examples of oncogenic fusion transcription factors that result from fusion of one chromosomal region with another such as to create a hybrid or fusion protein containing the DNA-binding domain of the transcription factor and another domain or sequence from the translocation partner. From PMID: 33634124

FIG. 15 provides examples of FLI1 ligands that could be used to construct a TF-CIP that would recruit or induce proximity of the activated and translocated FLI1 fusion protein to the promoters of pro apoptotic genes activating the genes and killing the cancer cell with its own driver.

FIG. 16 provides examples of ERG1 ligands that could be used to construct a TF-CIP that would recruit or induce proximity of the activated and translocated ERG fusion protein to the promoters of pro apoptotic genes activating the genes and killing the cancer cell with its own driver.

FIG. 17 provides ligands for FEV, an ETS family transcription factor expressed only in neurons that make serotonin in the dorsal raphe of the human brain and which controls the production of the rate-limiting enzyme for serotonin synthesis, TPH2. To make a TF-CIP that increases serotonin production, these ligands are linked to ligands for suitable transcriptional activators also selectively expressed in the target neuronal population. The ligands were identified by the steps illustrated in FIG. 8. Several of these ligands also bind to the ERG protein by Surface plasmon resonance.

FIG. 18 provides examples of myc ligands that could be used to synthesize a TF-CIP that would bring the oncogenic driver, myc to the promoter of cell death genes by using a TF-CIP consisting of one of the myc ligands, a chemical linker and a ligand for BCL6 or FOXO3A which binds the promoter of pro apoptotic genes. This would activate these genes and kill the cancer cell with its own driver.

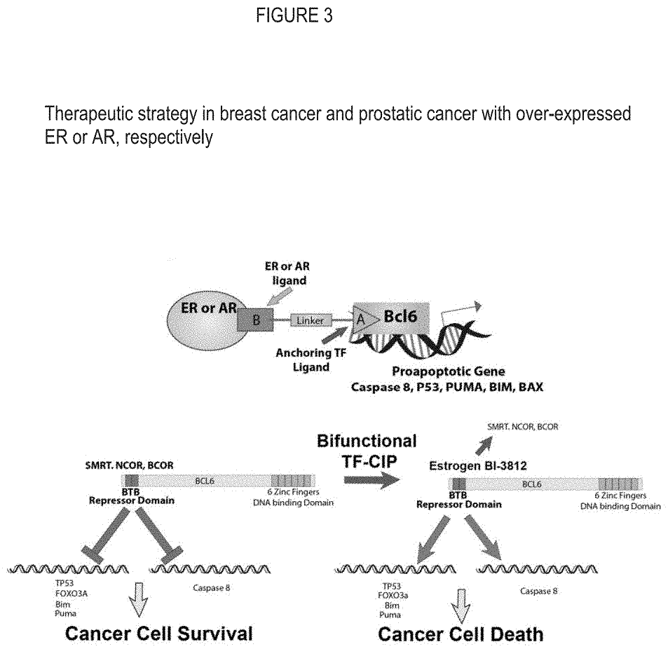

FIG. 19 provides an example of a ligand for the E2F transcription factor which may function as an anchor to recruit a repressor or may also function in other embodiments as a means of recruiting an activator to the promoter of a cell death gene as illustrated in FIG. 3.

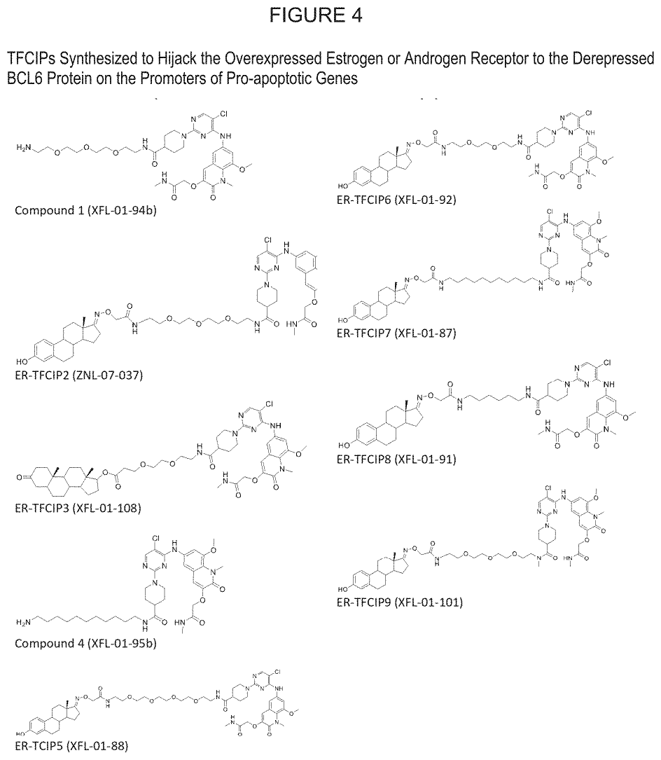

FIG. 20 provides examples of estrogen analogues that may be used to construct TF-CIPs similar to those shown in FIG. 4 to bring a cancer driver to the promoters of cell death genes or other genes in ER-positive breast cancer cells to alter or activate their expression and induce cell death in response to the tumor's driving mechanism, which in this specific example is the estrogen receptor. References for these estrogen analogues include: PMID: 12656587 DOI: 10.1021/ja0293305PMID: 15101754 DOI: 10.1021/oI0497537PMID: 2362442 DOI: 10.1016/0022-4731(90)90123-aPMID: 1780954D01: 10.1016/0039-128x(91)90070-cPMID: 3702438D01: 10.1016/0022-4731(86)90117-2PMID: 12794859DOI: 10.1002/cbic.200200499PM1D: 2738897 DOI: 10.1021/jm00127a040PMID:2064992 DOI: 10.1016/0960-0760(91)90090-rPMID: 12236347 DOI: 10.1021/ac020088u

FIG. 21 provides examples of agonist ligands for androgen receptors that could be used to bring the androgen receptor to the promoters of the cell death genes in prostatic cancer with amplification and overexpression of the androgen receptor. The synthesis of these ligands and their characteristics are described in: PMID: 30271980 PMCID: PMC6123676 DOI: 10.1038/s42003-018-0105-8PM1D: 10077001 DOI: 10.1210/mend.13.3.0255PMID: 16159155 PMCID: PMC2096617 DOI: 10.1021/cr020456uPMID: 24909511 PMCID: PMC4571323 DOI: 10.1038/aps.2014.18

FIG. 22 provides examples of ligands for the progesterone receptor that may be used to bring the progesterone receptor to the promoters of cell death (proapoptotic) genes. These are described in: PMID: 17013809 DOI: 10.1002/med.20083PMID: 26153859 PMCID: PMC4650274 DOI: 10.1038/nature14583

FIG. 23 provides examples of BAF53a ligands that may be used to activate cell death genes specifically in SCC when incorporated into a TF-CIP. These ligands may also be used as part of a TF-CIP that would be used to traverse developmental barriers by polycomb eviction and removal of developmental repression from lineage defining genes. Methods for identification of other suitable ligands is given in FIG. 8.

FIGS. 24A and 24B: Examples of chemical linker components that can be used to synthesize TF-CIPs. These components can be used to provide spacing and presentation of the anchoring transcription factor to the second transcription factor.

Linkers can be chosen and constructed from these and other published components to provide solubility to the TF-CIP compounds.

FIG. 25: Using TF-CIPs to restore the function of haploinsufficient genes by increasing transcription of the unmutated copy of the gene. The activating transcription factor can be chosen based on the availability of a suitable ligand.

FIG. 26: Panel A. Restoring the level of BAF250B in human neuroprogenitors using the FIRE Cas9 system(PMID 28916764) to bring a transcription factor to the promoter of the one unmutated allele of the haploinsufficient BAF250B (Arid1 B) gene. Panel B. After addition of the CIP, transcription is increased to the level of a wildtype neural progenitor. The rescue of transcription shown here by 2-fold should reverse the disease symptoms.

FIGS. 27 and 28: Chemical synthetic schemes to make TF-CIPs for activation of TPH2 and enhancement of serotonin production in cells of the dorsal raphe of the human brain. Each synthetic method begins with a ligand for FEV, for example those illustrated in FIG. 17, a chemical linker is then attached along with a ligand for Brd4 to generate a bifunctional FEV-TF-CIP capable of activation serotonin production in cells having a mutation in the TPH2 gene or one of the genes that controls TPH2. Other activating transcription factors can be chosen by screening as described for molecular glues in FIG. 9.

FIG. 29 provides an illustration of TF-CIPs activating expression of serotonin (Panel A) and dopamine (Panel B) synthesis in accordance with an embodiment of the invention.

### DETAILED DESCRIPTION

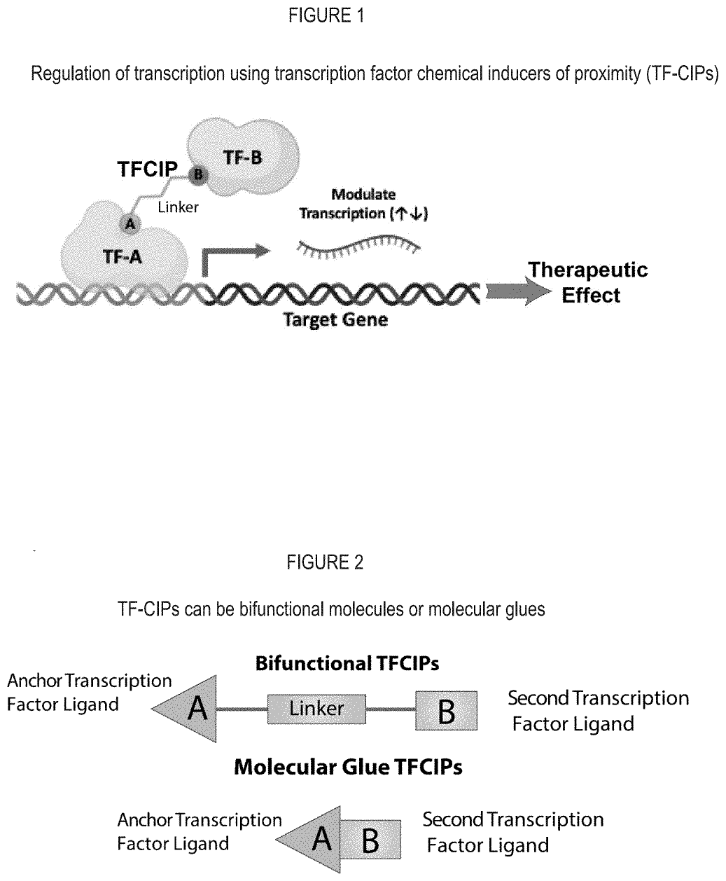

Methods of modulating transcription of a target gene in a cell (which may be in vitro or in vivo) are provided. Aspects of the methods employ a transcription factor-chemical inducer of proximity (TF-CIP) to modulate, e.g., enhance or reduce, transcription of a target gene in a cell (e.g., as illustrated in FIG. 1). Embodiments of the methods include providing in a cell a chemical inducer of proximity (CIP) which links a first endogenous anchor transcription factor that binds to a promoter of the target gene to a second endogenous transcription modulating factor (e.g., a transcription factor or transcription repressor), wherein CIP mediated linkage of the anchor transcription factor and transcription modulating factor regulates transcription of the target gene in the cell. Also provided are compositions that find use in practicing methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods and Chemical Inducers of Proximity (CIP)

As summarized above, aspects of the invention include methods of modulating transcription of a target gene in a cell. The methods may be viewed as inducible methods of modulating transcription of a target gene. As the methods are inducible, the modulation of transcription of the target gene is not constitutive, but instead occurs in response to an applied stimulus, e.g., the provision of a CIP, such as described in greater detail below. As the methods are methods of inducibly modulating transcription of a target gene, they are methods of changing transcription of a target gene in some manner, e.g., enhancing transcription of a target gene or reducing transcription of a target gene. The magnitude of change in transcription (relative to a suitable control, e.g., an identical system but for the absence of a CIP), may vary, where in some instances the magnitude of the change, e.g., enhancement or reduction, is 2-fold or greater, such 5-fold or greater, e.g., 10-fold or greater.

As summarized above, aspects of the invention include methods of modulating transcription of a target gene. The term gene refers to a genomic region that encodes a functional RNA, including non-coding RNAs, microRNAs, enhancer RNAs or RNAs that may be translated into a protein product. The term gene is used in its conventional sense to refer to a region or domain of a chromosome that includes not only a coding sequence, e.g., in the form of exons separated by introns, but also regulatory sequences, e.g., enhancers/silencers, promoters, terminators, non-coding RNAs, micro RNAs etc.

The specific target gene that is the focus of a given method may vary. In some instances, the target gene is a gene whose expression is to be enhanced, such as a pro-apoptotic gene (e.g., PUMA (BBC3), BIM (BCL2L11), BID, BAX, BAK, BOK, BAD, HRK, BIK BMF, and NOXA(PMAIP1), or a gene whose activity is inhibited such as the anti-apoptotic gene BCL6, a beneficially therapeutic gene (e.g., a rate-limiting enzyme (such as TPH2), a haploinsufficient gene (such as ARID1B), etc. In some instances, the target gene is an over-expressed gene whose expression is to be reduced, e.g., an oncogene (such as MYC), a trisomy gene (such as a chromosome 21 gene), or an amplified gene etc.

The above categories of genes are merely exemplary of the types of genes that may be target genes of the subject methods. Additional examples of target genes include, but are not limited to: developmental genes (e.g., adhesion molecules, cyclin kinase inhibitors, cytokines/lymphokines and their receptors, growth/differentiation factors and their receptors, neurotransmitters and their receptors); oncogenes (e.g., ABLI, BCLI, BCL2, BCL6, CBFA2, CBL, CSFIR, ERBA, ERBB, EBRB2, ETSI, ETS1, ETV6, FOR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3, and YES); tumor suppressor genes (e.g., APC, BRCA 1, BRCA2, MADH4, MCC, NF 1, NF2, RB 1, TP53, and WTI); enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, Upases, lipoxygenases, lyso/ymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases); chemokines (e.g. CXCR4, CCR5), the RNA component of telomerase, vascular endothelial growth factor (VEGF), VEGF receptor, tumor necrosis factors nuclear factor kappa B, transcription factors, cell adhesion molecules, Insulin-like growth factor, transforming growth factor beta family members, cell surface receptors, RNA binding proteins (e.g. small nucleolar RNAs, RNA transport factors), translation factors, telomerase reverse transcriptase); and the like.

FIG. 1 provides an illustration of a general embodiment of regulating transcription of a therapeutic gene using a TF-CIP in accordance with embodiments of the invention. As shown in FIG. 1, a TF-CIP (A-linker-B) includes a ligand A that specifically binds to an anchor transcription factor and a ligand B that specifically binds to a modulating, e.g., activator, transcription factor which is recruited (i.e., hijacked or rewired) to regulate transcription of the target gene. Binding of the TF-CIP to both the modulating transcription factor and the anchor transcription factor results in the production of a binding complex by virtue of its two binding moieties A and B joined by a chemical linker and activation of transcription of the target therapeutic gene. FIG. 1 also provides an illustration of general embodiment of repressing transcription of a therapeutic gene using a TF-CIP in accordance with embodiments of the invention. As shown in FIG. 1, a TF-CIP (A-linker-B) including a ligand A that specifically binds an anchor transcription factor which regulates transcription of the target gene and a ligand B that specifically binds to a transcription repressor factor . Binding of the TF-CIP to both the transcription repressor factor and the anchor transcription factor results in the production of a binding complex that represses transcription of the target gene.

### Chemical Inducers of Proximity (CIP)

As reviewed above, embodiments of the methods employ a Chemical Inducer of Proximity (CIP). A CIP is a compound that induces proximity of a first endogenous anchor transcription factor that binds to a promoter of the target gene and a second endogenous transcription modulating factor under intracellular conditions. As the CIPs of the invention induce proximity of at least one endogenous transcription factor with another endogenous transcription modulating factor (e.g., a transcription factor or transcription repressor), the CIPs of the invention may be referred to as Transcription Factor-Chemical Inducers of Proximity (TF-CIP) and are generally illustrated in FIGS. 2 and 3. By “induces proximity” is meant that the first and second endogenous factors are spatially associated with each other through a binding event mediated by the CIP compound (PMID: 29590011), which is configured to simultaneously bind to both endogenous factors, such that the CIP compounds may be viewed as a bifunctional compound or a molecular glue (PMID: 33417864). Spatial association is characterized by the presence of a binding complex that includes the CIP, first endogenous anchor transcription factor, the second endogenous transcription modulating factor (e.g., a transcription factor, transcription repressor, chromatin regulator, epigenetic regulator and/or cancer driver). In the binding complex, each member or component of the binding complex is bound to at least one other member of the complex. In this binding complex, binding amongst the various components may vary. For example, the CIP may simultaneously bind to domains of the first and second endogenous factors, thereby producing the binding complex and desired spatial association, e.g., which ultimately results in the desired transcription modulating of the target gene. This binding complex may be referred to a tripartite complex as it is made up of three distinct, non-covalently bound components, i.e., the endogenous anchor transcription factor, the endogenous transcription modulating factor and the TF-CIP.

Any convenient compound that functions as a CIP may be employed. A wide variety of compounds, including both naturally occurring and synthetic substances, can be used as CIPs. Applicable and readily observable or measurable criteria for selecting a CIP include: (A) the ligand is physiologically acceptable (i.e., lacks undue toxicity towards the cell or animal for which it is to be used); (B) it has a reasonable therapeutic dosage range; (C) it can cross the cellular and other membranes, as necessary, and (D) binds to the target domains of the endogenous anchor transcription factor and the endogenous transcription modulating factor. As such, a desirable criterion is that the compound is relatively physiologically inert, but for its CIP activity. In some instances, the ligands will be non-peptide and non-nucleic acid. Of interest in some applications are compounds that can be taken orally (e.g., compounds that are stable in the gastrointestinal system and can be absorbed into the vascular system).

CIP compounds of interest include small molecules and are non-toxic. By small molecule is meant a molecule having a molecular weight of 5000 g/mole (Dalton) or less, such as 2500 g/mole (Dalton) or less, including 1000 g/mole (Dalton) or less, e.g., 500 g/mole (Dalton) or less. In some instances, the CIP employed in embodiments of the invention has a molecular weight ranging from 250 to 1500 g/mole, such as 300 to 1200 g/mole. By non-toxic is meant that the inducers exhibit substantially no, if any, toxicity at concentrations of 1 g or more/kg body weight, such as 2.5 g or more /kg body weight, including 5g or more/kg body weight.

CIP compounds employed in embodiments of the invention include a first ligand that specifically binds to the anchor transcription factor covalently linked to a second ligand that specifically binds to the transcription modulating factor. In other words, the CIP compounds include a linker component, which may be a bond or a linking moiety, which links a first ligand that specifically binds to the anchor transcription factor covalently and a second ligand that specifically binds to the transcription modulating factor. The terms “specific binding,” “specifically bind,” and the like, refer to the ability of the first and second ligands to preferentially bind directly to their corresponding anchor and transcription modulator factors relative to other molecules or moieties in the cell. In certain embodiments, the affinity between a given ligand and its corresponding factor when they are specifically bound to each other in a binding complex is characterized by a KD (dissociation constant) of 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, or 10⁻¹⁵ M or less (it is noted that these values can apply to other specific binding pair interactions mentioned elsewhere in this description, in certain embodiments).

The nature of the first and second ligands, as well as the linker components, of the CIP compounds may vary. In any given CIP compound, the first and second ligands will be chosen based on the nature of their corresponding anchor and transcription modulating factors, where examples of such and their corresponding ligands are provided below. Specificity of activity with respect to a particular cell type may be provided through selection of the first and second ligands of the CIP, which can be configured to recruit anchor transcription factors and transcription modulatory factors in a manner that provides for desired cell or conditional specificity. For example, CIPs can be engineered to induce proximity of anchor transcription factors and transcription modulatory factors that are primarily present in a target cell of interest, such that the CIP exhibits highly selective activity for that cell. The selectivity of a given CIP may be described by the following formula:

(selectively of expression of anchor transcription factor)×(selectively of expression of the transcription modulating factor)×(genomic specificity of anchor transcription factor)=selectivity of induced activity

### Production of TF-CIPs by Rational Design Using Existing Components

FIG. 3 provides an illustration of how to build a TF-CIP by rational design using existing components. As shown in FIG. 3, the design of a TF-CIP configured to hijack BCL6 to kill ER-positive breast cancer cells or AR-positive prostatic cancer cells is shown. BCL6 is a transcription factor and oncogene that prevents death of a variety of cancer cells, including breast cancer cells, by binding epigenetic the repressors BOOR, NCO and SMRT (PMID:30335946) on the promoters of cell death genes. Several inhibitors of BCL6's repressive function have been produced that prevent the binding of BOOR, NCOR and SMRT to a site formed by the dimeric surface of BCL6 (PMID:18280243) However, these inhibitors have not been sufficiently active to be used therapeutically (PMID:30335946).

Chemical linkage of BCL6 inhibitors, such as B13812(PMID32275432), to estrogen compounds that then bind and induce proximity to the cell death (pro-apoptotic) promoters, such as those for TP53, PUMA and BIM, convert the inhibitor of cell death to an activator of cell death in cells having high concentrations of estrogen receptors, such as breast cancer cells. Examples of the TF-CIPs synthesized by the above protocol and designed to hijack BCL6's repressive activity and convert it to an activator of cell death are shown in FIG. 4. Details of the synthesis of the compounds are provide in Example 1, section H, below. Each structure includes an estrogen receptor binding moiety connected by a chemical linker to a BCL6 inhibitor based on the previously described molecule B13812 (PMID32275432) . These types of molecules may find use in treating ER-positive breast cancer.

Also shown is an example of a structure using a similar strategy employing an androgen analogue linked to a BCL6 inhibitor based on the previously described molecule B13812(PMID32275432). The later type of molecule may find use in treating prostatic cancer where the AR gene is amplified or overexpressed.

FIGS. 5A to 5C provide examples of how one measures the effect of a TF-CIP to allow chemical optimization of linker and ligands. In the illustrated embodiment, the reporter includes of an array of BCL6 binding sites taken from different pro-apoptotic genes, including TP53, PUMA, BIM and others. These genes have the ability to kill cells when simply overexpressed (PMID11463391). Ten base pairs on either side of the actual human BCL6 binding site are included to take advantage of the fact that transcriptional specificity is due to the concerted binding of proteins in an enhanceosome (PMID:9510247 PMID:33957125, PMID 1179502). Thus, the reporter system is multiplexed and useful for defining and quantitating apoptotic responses in many different cell types. The second reporter, which is used for providing mechanistic information about the specificity and action of the first reporter and is also able to quantitatively assess a different group of cell death processes includes an array of FOXO3A binding sites taken from different pro-apoptotic genes including TP53, PUMA, BIM and others. These genes have the ability to kill cells when simply overexpressed or when FOXO3A is overexpressed (PMID11463391). Ten base pairs on either side of the actual human FOXO3A binding site are included to take advantage of the fact that transcriptional specificity is due to the concerted binding of proteins in an enhanceosome (PMID:9510247, PMID:18206362). Thus, this reporter system is multiplexed and useful for defining apoptotic responses in many different cell types.

These reporter systems and direct measures of cancer cell killing were used to assess the TF-CIP molecules made by rational design that are illustrated in FIG. 4. The results of evaluation and the relative potency and mechanism of action of TF-CIPs is shown in FIG. 6. As shown in Panel A, addition of ER-TF-CIP6 activates GFP expression by the BCL reporter with an EC50 of about 1 to 10 micromolar. This is a substantially lower concentration than that of the parent compound, BI3812 required to produce phenotypes in published studies. Note that the higher concentrations lead to a reduction in activation of the reporter consistent with the “hook effect” characteristic of bifunctional molecules as they saturate both binding sites. (PMID: 8752278; PMID:21406691), in this case the BCL6 protein and the estrogen receptor. Panel B shows that ER-TF-CIP6 induces death of cancer cells containing an activated rearranged BCL6 gene and a mildly overexpressed estrogen receptor (in this case Karpas 422) more effectively than B13812. Panel C demonstrates that ER-TF-CIP6 induces cell death in HEK293 cells. Panels D and E show that breast cancer cell lines that do not express the estrogen receptor or express it at low levels are not killed by ER-TF-CIP6. These studies show that ER-TF-CIP6 and other TF-CIPs shown in FIG. 4 recruit the estrogen receptor to the disinhibited BCL6 to activate pro apoptotic genes that then kill the cells. Viability was measured using the PrestoBlue HS (resazurin) assay for viable cells (1:10 ratio in media and 1 h incubation).

The therapeutic effectiveness of ER-TF-CIPs can be improved in several ways. First, the estrogen analogue can be chosen from many published estrogen analogues, including those that have a higher affinity for the estrogen receptor than estrodiol (DOI: 10.1002/cbic.200200499; PMID:12794859; PMID:15300835; PMID:2064992 PMID; 2362442; PMID:3702438). The latter would have certain advantages in treating women who are not post menopausal and have high levels of estrogen that could compete with the ER-TFCIP. The therapeutic effectiveness of an ER-TFCIP can be improved by use of different linkers which will be discussed and illustrated in more detail later in the application. Different linkers could position the Estrogen Receptor more effectively to the BCL6 protein, or could give the ER-TF-CIP superior pharmacologic features. The therapeutic effectiveness of an ER-TFCIP can be improved by use of different BCL6 ligands, some of which are illustrated in the following publications: (PMID:32275432; PMID:30335946; PMID:28930682; PMID:27482887).

### Production of TF-CIPs by Rational Desicin Using Novel Components

The studies described in the section above teach one how to build and evaluate a TF-CIP made from existing components. In cases where existing components or the ligands for the anchor and regulatory transcription factors are not available, the following methods to detect and evaluate these novel ligands may be employed. FIG. 7A provides a step-by-step illustration of how to pick pairs of transcription factors selectively expressed in the target tissue of interest. From this analysis, anchor transcription factors for a specific target gene of interest are identified using the step-by-step instructions in FIG. 7B. In this representative example, the anchoring transcription factors for treatment of breast cancer are illustrated. Considerations of final selection for the anchor transcription factor include specificity of expression in the target tissue, documented role in the biologic process to be enhanced or repressed and clear indication of the importance of the binding site for the transcription factor. Application of these principals provides both an activating or repressing transcription factor selectively expressed in the target tissue of interest and one or more candidate anchor transcription factors, also expressed in the target tissue of interest, which then can be used for selection of ligands.

A protocol for selecting a ligand to be used in a TC-CIP for an anchor transcription factor is provided using the protocol illustrated in FIG. 8. Selection of a pocket within the anchoring TF involves the use of Site Map PMID: 19434839 for example, but other programs can also be used. Docking to the pocket and scoring of hits are described in PMID: 17034125 and PMID: 15027866. Other methods of selecting ligand include DNA-encoded library screening (PMID:28094476) with the protein of interest. Yet another way of selecting ligands is by screening libraries of small molecules using fluorescence polarization or other direct methods of measuring binding. Yet another way of detecting ligands is using nanoBRET(PMID 30972335).

An additional way that a totally novel TF-CIP can be detected in a library of small molecules involves the use of the reporters shown in FIG. 9. Described in FIG. 9 is a screen for TF-CIPs that act as molecular glues using the reporters introduced in FIGS. 5A to 5C. A “molecular glue” is distinguished from a “bifunctional molecule”, such as those seen in FIG. 2, by virtue of the fact that the linker has been replaced with a more direct connection between the two binding moieties. Molecular glues, such as FK506 or rapamycin (PMID:33436864), often have good pharmacologic behavior. Most importantly, they can engage several proteins, e.g., as illustrated in FIG. 9, at once by promoting interactions between the target protein, such as a cancer driver, and several proteins within a highly biologically specific enhanceosome(PMID:9510247). Shown are the reporters for use in detecting inhibitors of BCL6 (upper panel) as well as activators of FOXO3A (lower panel). Molecules which act as molecular glues can be selected from large libraries of compounds by virtue of their ability to activate GFP or mCherry expression in a given cell type, for example breast cancer cells, prostatic cancer cells or lymphomas.

### Production of TF-CIPs: Additional Design Considerations

In some instances, the first and second ligands of the CIPs are small molecules, which in some instances each have a molecular weight ranging from 50 Daltons to 1000 Daltons, such as to 400 to 800 Daltons. The chemical structures of the first and second ligands may vary widely, where the first and second ligands may be chosen to provide for the desired specific binding to the target anchor transcription or transcription modulatory factors. The first and second ligands may be selected so as to have little, if any, impact on the activity of the endogenous factor, e.g., anchor transcription factor or transcription modulating factor, to which they are configured to bind.

For a given target gene, anchor transcription factors and ligands therefore that may be employed in a TF-CIP may be identified using any convenient protocol. In some instances, a protocol as described in FIGS. 7A to 7C may be employed to identify an anchor transcription factor for a target gene of interest. Once a suitable transcription factor is identified, a ligand therefore that can be used in a TF-CIP may be identified using a protocol as described in FIG. 8.

As described above, the first and second ligands of the CIPs may be bound to each other by a bond, or via a linking moiety, i.e., linker. When employed, any convenient linker may be employed to link the first and second ligands to each other. Linkers of interest are linkers that provide for a stable association of the first and second ligands in a manner such that the first and second ligands are capable of specifically binding to their respective endogenous factors in the cell. As the linker provides for stably associating the first and second ligands with each other, the first and second ligands do not dissociate from each other under cellular conditions, e.g., conditions at the surface of a cell, conditions inside of a cell, etc. Linkers may be provided for stable association of the first and second ligands using any convenient binding, such as covalent or non-covalent binding, where in some instances the linker component is covalently bound to both the first and second ligands.

Where a CIP includes a linker covalently bound to the first and second ligands, any convenient protocol for forming a covalent bond between the linker and each of the ligands may be employed, where linking protocols of interest include, but not limited to, addition reactions, elimination reactions, substitution reactions, pericyclic reactions, photochemical reactions, redox reactions, radical reactions, reactions through a carbene intermediate, metathesis reaction, among other types of bond-forming reactions. In some embodiments, the linkers employ reactive linking chemistry such as where reactive linker pairs (e.g., as provided by moieties on the ligands and linkers) include, but are not limited to: maleimide/thiol; thiol/thiol; pyridyldithiol/thiol; succinimidyl iodoacetate/thiol; N-succinimidylester (NHS ester), sulfodicholorphenol ester (SDP ester), or pentafluorophenyl-ester (PFP ester)/amine; bissuccinimidylester/amine; imidoesters/amines; hydrazine or amine/aldehyde, dialdehyde or benzaldehyde; isocyanate/hydroxyl or amine; carbohydrate—periodate/hydrazine or amine; diazirine/aryl azide chemistry; pyridyldithiol/aryl azide chemistry; alkyne/azide; carboxy-carbodiimide/amine; amine/Sulfo-SMCC (Sulfosuccinimidyl 4-[N-maleimidomethyl]cyclohexane-1-carboxylate)/thiol and amine/BMPH (N-[β-Maleimidopropionic acid]hydrazide.TFA)/thiol; azide/triarylphosphine; nitrone/cyclooctyne; azide/tetrazine and formylbenzamide/hydrazino-nicotinamide. In certain embodiments, a linker employs a cycloaddition reaction, such as a [1+2]-cycloaddition, a [2+2]-cycloaddition, a [3+2]-cycloaddition, a [2+4]-cycloaddition, a [4+6]-cycloaddition, or cheletropic reactions, including linkers that undergo a 1,3-dipolar cycloaddition (e.g., azide- alkyne Huisgen cycloaddition), a Diels-Alder reaction, an inverse electron demand Diels Alder cycloaddition, an ene reaction or a [2+2] photochemical cycloaddition reaction. In some embodiments, the linker may include an alkyl chain, an alkoxy chain, an alkenyl chain or a alkynyl chain, where the number of carbon atoms in the chain may vary, ranging in some instances from 2 to 25, such as 5 to 20, where one or more carbon atoms are replaced with NH or CH₃—N as reactive functionalities for covalent bonding.

In some instances, the linker is selected from a group comprising the following, where n refers to the total number of carbon or carbon-substituent atoms which may be present, sub-counted by k, m, and/or p:
a) A Cn alkyl chain, L, including the case where one or more carbon atoms are replaced with NH or CH₃—N
b) A Cn alkoxy chain, L, including the case where one or more carbon atoms are replaced with NH or CH₃—N
c) A Cn alkenyl or alkenyloxy chain, L, including the case where one or more carbon atoms are replaced with NH or CH₃—N
d) A Cn alkynyl or alkynyloxy chain, L, including the case where one or more carbon atoms are replaced with NH or CH₃—N
e) L¹-Ar-L² or L¹-Het-L², where L¹ and L² can be a bond, alkenyl, alkynyl, alkynyloxy, alkenyloxy, alkoxy, or alkyl chain of 1-10 atoms that are either carbon or optionally substituted nitrogens, such as CH₂N(H)CH₂, CH₂OCH₂, C₅H₁₀OCH₂, and others (see FIG. 4); Ar is a 6 membered optionally substituted aryl; and Het is a 4 to 6 membered heterocycloalkyl or a 9 to 10 membered spirocyclic bicyclic heterocycloalkyl or a 3 to 6 membered optionally substituted heteroaryl.

The structures of linker molecules and non-inclusive selected examples are shown in FIGS. 24A and 24B. Specific linkers that may be employed in embodiments of the invention include, but are not limited to, those depicted below:

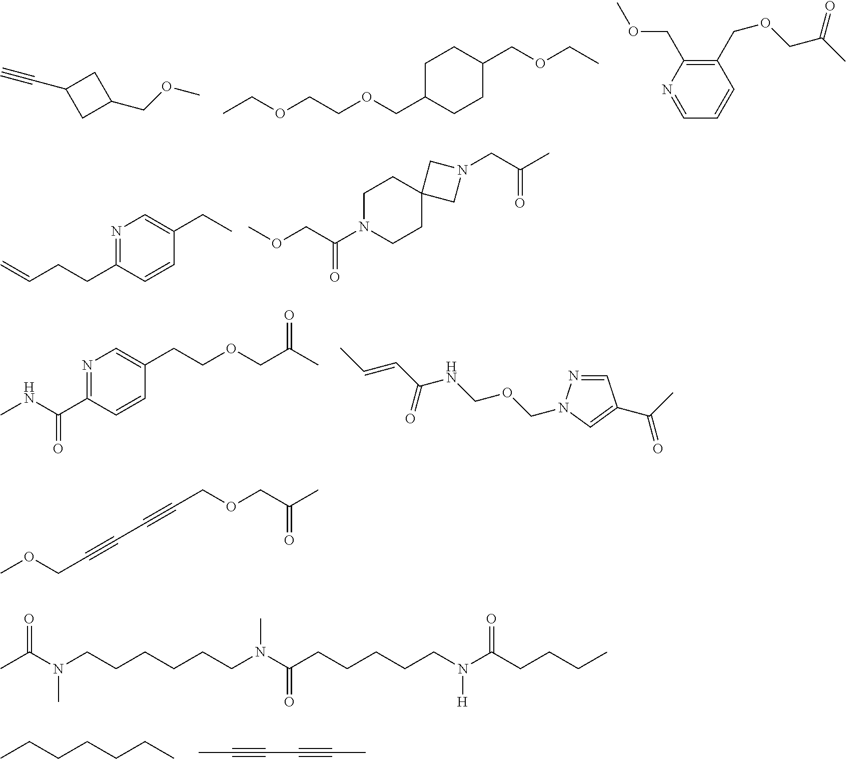

### Endogenous Anchor Transcription Factor

As summarized above, the CIP employed in embodiments of the invention includes a first ligand that specifically binds to an endogenous anchor transcription factor that binds to a promoter of a target gene, e.g., as illustrated in FIGS. 1 and 2 and described above. The term “transcription factor” is employed in its conventional sense to refer to a protein that controls the rate of transcription of genetic information from DNA to a transcribed RNA product, e.g., messenger RNA, non-coding RNA, etc., by binding to a specific DNA sequence, e.g., through interaction of a DNA binding domain with a transcription factor-binding site or response element. Transcription factors may also be referred to as sequence-specific DNA-binding factors.

Anchor transcription factors of the invention are those transcription factors that bind to a transcription factor-binding site or response element of the target gene of the cell and modulate, e.g., enhance or repress, transcription thereof. As the anchor transcription factors are endogenous, they originate from the cell and are not heterologous to the cell. As such, an anchor transcription factor of a cell in which a method of invention is carried out is one that is encoded by a chromosomal gene, where the chromosomal gene is not heterologous to the cell, i.e., the gene has not been introduced into the chromosome of the cell, e.g., by a vector, such as a viral vector or has been genetically modified in anyway.

The endogenous anchor transcription factor may vary widely depending on the nature of the particular target gene and type of modulation, e.g., transcription enhancement or reduction, desired. Examples of anchor transcription factors that may be employed in embodiments of the invention include, but are not limited to: general transcription factors that are involved in the formation of a preinitiation complex, e.g., TFIIA, TFIIB, TFIID, TFIIE, TFIIF, and TFIIH; and upstream transcription factors, e.g., proteins that bind somewhere upstream of the initiation site to stimulate or repress transcription. Endogenous anchor transcription factors employed in given embodiments of the invention may be any of the transcription factors described in Lambert et al., “The Human Transcription Factors,” Cell (February 8, 2018) 172: 650-665 as well as those listed in the supplementary materials therefore and also at http://humantfs.ccbr.utoronto.ca/. Specific anchor transcription factors finding use in exemplary applications of the invention are reviewed in greater detail below.

### Endogenous Transcription Modulatory Factor

As summarized above, the CIP employed in embodiments of the invention includes a second ligand that specifically binds to an endogenous transcription modulatory factor, e.g., as illustrated in FIGS. 1 and 2 and described generally above. The endogenous transcription modulatory factor is a protein that is endogenous to the cell and not genetically modified other than by nature, as described above with respect to the endogenous anchor transcription factor. The endogenous transcription modulatory factor is a protein that, when present in a binding complex with the CIP and anchor transcription factor, modulates transcription of the target gene in a desirable manner, e.g., enhances or reduces transcription of the target gene. Transcription modulatory factors may vary, where examples of such include transcription factors, e.g., as described above, as well as transcription modulatory proteins that do not bind directly to DNA. Transcription modulatory proteins that do not bind directly to DNA which may be employed in embodiments of the invention include, but are not limited to: heterochromatin formation mediators, such as: mediators of histone methylation or demethylation, DNA methylation or demethylation, nucleosome bridging, histone acetylation or deacetylation, histone phosphorylation or dephosphorylation, histone ubiquitination or deubiquitination, contact between DNA and histones, etc. Specific mediators of interest include, but are not limited to: HP1 proteins, e.g., HP1a and cs HP1a, histone H3K9 methylases, histone H3K9 demethylases, histone H3K27 methylases, histone H3K27 demethylases, histone H3K4 methylases such as MLL, histone H3K4 demethylases, histone acetyltransferases, histone deacetyltransferases, etc. In some instances, the transcription modulatory factor that does not bind to DNA is a transcription repressor protein, e.g., heterochromatin protein 1 (HP1) repressor proteins, KRAB repressor proteins, etc. Specific transcription modulatory factors finding use in exemplary applications of the invention are reviewed in greater detail below. In other cases, the transcriptional modulator may be an ATP-dependent chromatin regulator such as BAF or mSWI/SNF, PBAF, INO80 or LSH1 or any subunits contained withing their complexes. In some instances, one of the approximately 30 ATP-dependent chromatin regulators encoded in the mammalian genome that are similar to BRG1 (SMARCA4) or BRM (SMARCA2) or their subunits and associated proteins may be used for recruitment to specifically alter Polycomb Repressive Complexes or other chromatin features in embodiments of the invention.

### Cells

As summarized above, aspects of methods of invention include providing a CIP in a cell in which transcription of a target gene is to be modulated. The cell that is provided with the CIP compound may vary depending on the specific application being performed. Cells of interest include eukaryotic cells, e.g., animal cells, where specific types of animal cells include, but are not limited to yeast, insect, worm or mammalian cells. Various mammalian cells may be used, including, by way of example, equine, bovine, ovine, canine, feline, murine, non- human primate and human cells. Among the various species, various types of cells may be used, such as hematopoietic, neural, glial, mesenchymal, cutaneous, mucosal, stromal, muscle (including smooth muscle cells), spleen, reticulo- endothelial, epithelial, endothelial, hepatic, kidney, gastrointestinal, pulmonary, fibroblast, and other cell types. Hematopoietic cells of interest include any of the nucleated cells which may be involved with the erythroid, lymphoid or myelomonocytic lineages, as well as myoblasts and fibroblasts. Also, of interest are stem and progenitor cells, such as hematopoietic, neural, stromal, muscle, hepatic, pulmonary, gastrointestinal and mesenchymal stem cells, such as ES cells, epi-ES cells and induced pluripotent stem cells (iPS cells). As summarized above, the cells that are provided with the CIP compounds contain at least the endogenous anchor transcription factor and endogenous transcription modulatory factor. As such, the cells are cells that naturally include the anchor transcription factor and transcription modulatory factor and have not been engineered to include these factors. As desired, the cells may be in vitro or in vivo. In some instances, the cell in which transcription of the target gene is to be modulated is part of a multicellular organism.

### Methods Steps

Aspects of the invention include providing the CIP in the cell, e.g., as described above, in a manner sufficient to induce proximity of the anchor transcription factor and transcription modulatory factor, e.g., as described above. Any convenient protocol for providing the CIP in the cell may be employed. The particular protocol that is employed may vary, e.g., depending on whether the target cell is in vitro or in vivo. In certain instances, the CIP is provided in the cell by contacting the cell with the CIP. For in vitro protocols, contact of the CIP compound with the target cell may be achieved using any convenient protocol. For example, target cells may be maintained in a suitable culture medium, and the CIP compound introduced into the culture medium as described specifically in the figures.

For in vivo protocols, any convenient administration protocol may be employed. Depending upon the binding affinity of the CIP compound, the response desired, the manner of administration, the half-life, the number of cells present, various protocols may be employed. Thus, the CIP can be incorporated into a variety of formulations, e.g., pharmaceutically acceptable vehicles (also referred to herein as pharmaceutical delivery vehicles or carriers), for therapeutic administration. More particularly, the CIP of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments (e.g., skin creams), solutions, suppositories, injections, inhalants and aerosols. As such, administration of the agents can be achieved in various ways, including oral, buccal, rectal, parenteral, intraperitoneal, intradermal, transdermal, intracheal, etc., administration. In pharmaceutical dosage forms, the CIPs may be administered alone or in appropriate association, as well as in combination, with other pharmaceutically active compounds. The following examples are illustrative and not limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill in the art will readily appreciate that dose levels can vary as a function of the specific compound, the nature of the delivery vehicle, and the like. Preferred dosages for a given compound are readily determinable by those of skill in the art by a variety of means.

In those embodiments where an effective amount of an active agent is administered to a living subject, the amount or dosage is effective when administered for a suitable period of time, such as one week or longer, including two weeks or longer, such as 3 weeks or longer, 4 weeks or longer, 8 weeks or longer, etc., so as to evidence a desired therapeutic effect. For example, an effective dose is the dose that, when administered for a suitable period of time, such as at least about one week, and maybe about two weeks, or more, up to a period of about 3 weeks, 4 weeks, 8 weeks, or longer, will results in a desired therapeutic effect. In some instances, an effective amount or dose of active agent will not only slow or halt the progression of the disease condition but will also induce the reversal of the condition, i.e., will cause an improvement one or more symptoms of the condition. For example, in some instances, an effective amount is the amount that when administered for a suitable period of time, usually at least about one week, and maybe about two weeks, or more, up to a period of about 3 weeks, 4 weeks, 8 weeks, or longer will improve one or more symptoms of a subject suffering from a disease condition, where the magnitude of improvement (e.g., as measured using a suitable protocol with relevant control) may vary, for example 1.5-fold, 2-fold, 3-fold, 4-fold, 5-fold, in some instances 6-fold, 7-fold, 8-fold, 9-fold, or 10-fold or more. In certain embodiments, the methods include removing the CIP from the cell at some point after provision of the CIP. Removal of the CIP from the cell may be accomplished using any convenient protocol, e.g., by removing the CIP from the medium in which the cell is present, by ceasing administration of the CIP to the animal comprising the cell, by contacting the cell with an inhibitor of the CIP induced proximity, by contacting the cells with a molecule that displaces the CIP and binds to only one of the endogenous anchor transcription or transcription modulatory factors, etc. One specific type of inhibitor of the action of the TF-CP would be a one-sided molecule consisting of the ligand for either the anchor or the hijacked transcription factor without the linker or other moiety.

As summarized above, aspects of the invention further include methods of inducibly modulating transcription of a target gene. Such methods include providing a chemical inducer of proximity (CIP) in a cell (e.g., a eukaryotic cell) containing an endogenous anchor transcription factor and endogenous transcription modulatory factor, e.g., as described above, under conditions sufficient to modulate transcription of the target gene. The CIP and cell may be as described above. The transcription modulation may vary. In some instances, the modulating includes enhancing transcription of the gene, e.g., where the gene is beneficial with respect to the disease condition, e.g., by enhancing a desired activity in the cell, such increasing expression of a proapoptotic gene where death of the cell is desired, increasing expression of a therapeutically beneficial gene where increased amounts of the product of such gene are beneficial with respect to a given disease condition, etc. In such instances, the magnitude of enhancement may vary, where examples include from substantially no to some expression, and in some instances the magnitude may be 2-fold or greater, such a 5-fold or greater, including 10-fold or greater. In some instances, the modulating includes reducing transcription of the target gene, e.g., where the gene is harmful, e.g., c-myc or a triplet expansion gene, e.g., such as Huntington, etc. In such instances, the magnitude of reduction may vary, where examples include from some expression to substantially none, if any, expression, and in some instances the magnitude of reduction may be 2-fold or greater, such a 5-fold or greater, including 10-fold or greater.

In some instances, the cell is a cell of a subject suffering from a disease condition, i.e., a cell obtained from such a subject or a cell that is part of such a subject. Disease conditions from which the subject may be suffering may vary, where examples of such disease conditions include, but are not limited to: neoplastic disease conditions, e.g., cancers; neurological conditions, immune disorders, gastrointestinal diseases, cardiovascular diseases and the like.

The subject methods find use in the treatment of a variety of different conditions in which the modulation of target gene transcription in a host is desired. By treatment is meant that at least an amelioration of one or more of the symptoms associated with the condition afflicting the host is achieved, where amelioration is used in a broad sense to refer to at least a reduction in the magnitude of a parameter, e.g., symptom, associated with the condition being treated. As such, treatment also includes situations where the pathological condition, or at least symptoms associated therewith, are completely inhibited, e.g., prevented from happening, or stopped, e.g., terminated, such that the host no longer suffers from the condition, or at least the symptoms that characterize the condition.

Where the methods are methods of treating a subject for a condition, the methods may further include assessing that the subject has the given condition, e.g., so as to confirm that a given CIP is suitable for use in treating the subject for the condition. Any convenient diagnostic protocol appropriate for a given condition may be employed, where the choice of such protocol will necessarily depend on the specific condition to be treated.

A variety of subjects are treatable according to the subject methods. In some instances, the subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some instances, the subjects are humans.

The following sections provide further details regarding illustrative embodiments of the methods of the invention.

### Enhancing Transcription of Pro-Apoptotic Genes

Embodiments of the invention include methods of enhancing transcription of a pro-apoptotic gene in a cell, e.g., as illustrated in FIG. 3. By enhancing transcription of a pro-apoptotic gene is meant increasing transcription of the pro-apoptotic gene. The magnitude of increase in transcription may vary. In those instances where transcription of the pro-apoptotic gene is not detectable by a suitable assay, embodiments of the methods result in an enhancement of transcription so that transcription is detectable, e.g., by detecting the expression product of the proapoptotic gene or activity thereof, e.g., apoptosis or an indicator thereof. In those instances where there is a base level of transcription that is detectable, the magnitude of increase may vary and, in some instances, may be 1.5-fold or more, 2-fold or more, such as 5-fold or more, including 10-fold or more.

The methods may result in enhancing transcription of a variety of different proapoptotic genes. Proapoptotic genes are genes the expression products of which promote or cause apoptosis, i.e., programmed cell death that occurs in multicellular organisms, which may be characterized by a variety of cell changes, such as blebbing, cell shrinkage, nuclear fragmentation, chromatin condensation, chromosomal DNA fragmentation, and global mRNA decay, and death. Specific proapoptotic genes of interest for transcription that may be enhanced in embodiments of the invention include, but are not limited to: PUMA (BBC3), BIM (BCL2L11), BID, BAX, BAK, BOK, BAD, HRK, BIK, BMF, and NOXA, and the like. Their relative ability to kill breast cancer cells when simply overexpressed are shown in Table 1. These measurements are useful in picking transcription factors and ligands for targeting the TF-CIP to a specific effective pro-apoptotic gene, such as BMF and HRK.

TABLE 1
Instructive Example of the Method of Selection of Pro-apoptotic Genes
for Hijacking Cancer Drivers to Intrinsic Cell Death Pathways

| | | % viable MCF7 | | % increase |
|---|---|---|---|---|
| | | cells after* | | in apoptotic |
| | | overexpression | | MCF7 cells |
| Gene | Class/Function | (24 h & 48 h) | | (24 h)** |
| BIM (BCL2L11) | BH3 activator | 66% | 42% | 23% |
| BID | BH3 activator | 100% | 89% | 6% |
| PUMA (BBC3) | BH3 activator/ | 98% | 90% | 8% |
| | sensitizer | | | |
| BAD | BH3 sensitizer | 100% | 98% | 17% |
| NOXA (PMAIP1) | BH3 sensitizer | 91% | 69% | 37% |
| HRK | BH3 sensitizer | 88% | 57% | 39% |
| BMF | BH3 sensitizer | 83% | 55% | 51% |
| BIK | BH3 sensitizer | 91% | 79% | 18% |
| BAX | Direct pore former | 100% | 87% | 19% |
| BAK | Direct pore former | 99% | 80% | 38% |
| BOK | Alternative pore | 97% | 90% | 32% |
| | former | | | |

*Values shown on the right are the actual experimental values found after inducing expression in MCF7 ER-positive breast cancer. (see experimental section)

Pro-apoptotic genes are of particular interest because they are expressed at levels that balance the anti-apoptotic genes, allowing the cell to survive by virtue of this balanced steady state.

Aspects of the methods of these embodiments include providing in the cell, e.g., via a protocol as described above, a chemical inducer of proximity (CIP) which links a first endogenous anchor transcription factor that binds to a promoter of the proapoptotic gene and a second endogenous oncogenic transcription factor, wherein CIP mediated linkage of anchor and oncogenic transcription factors enhances transcription of the proapoptotic gene in the cell. In some instances, CIPs employed in these embodiments are generally as described above and include a first ligand that specifically binds to the anchor transcription factor and a second ligand that specifically binds to the oncogenic transcription factor, where these first and second ligands are joined by a bond suitable linker, e.g., as described above.

A variety of different anchor transcription factors may be employed in methods of these embodiments. FIG. 7 provides a systematic way of defining an anchor transcription factors that is generalizable to any target gene, and may be employed to identify transcription factors of interest to target for a given pro-apoptotic gene. As illustrated in FIG. 7, the protocol starts by defining the region in the target gene available (i.e., accessible) for TF binding using existing resources, such as ATAC- or DNAse-seq. The identified accessible region is then assessed for transcription factors that are enriched in the cell type of interest, e.g., by using the Human Protein Altas (https://www.proteinatlas.org) or analogous resource, which provides specificity for the TF-CIP function and therapeutic specificity. In the lower panel of FIG. 7 are shown transcription factors that are enriched in breast cancer cells and that bind to the accessible regions of the cell death (pro-apoptotic) genes. Anchor transcription factors of interest include, but are not limited to: BCL6, TFAP2A, TFAP2C, SP3, TFDP1, ELK3, SREBF1, SREBF2, THRA, SMAD2, TFDP1, TCF3, USF1, USF2, VEZF1, PBX1, HIF1A, RARA, FOXO3A, MAZ, E2F1, E2F2, PAX9, STAT1, SPDEF, CREB3L1, BATF, XBP1, SIX4, AR, LEF1, MYB, RUNX1, and PPARG.

In TF-CIPs of these embodiments, any convenient ligand for these anchor transcription factors may be employed, where suitable ligands include small molecule ligands that are capable of specifically binding to the target anchor transcription factor without any relevant negative impact on the anchor transcription factor's ability to bind to target DNA binding site. The molecular weight of these ligands may vary, and in some instances ranges from 50 Daltons to 1200 Daltons such as 200 to 500 Daltons. A general method for identifying suitable ligands for use in such TF-CIPs is provided in FIG. 8. Suitable ligands for the anchor transcription factor may be chosen using any convenient protocol, such as in silico screening protocols, and the like, such as described below. For example, where the anchor transcription factor is FOXO3A, suitable ligands include, but are not limited to those shown in FIGS. 10 and 11 where those ligands for FOXO3A that bind a site near the DNA are shown in FIG. 10 and those that bind a site distal to the DNA are shown in FIG. 11. Where the anchor transcription factor is HIF1A, suitable ligands include, but are not limited to, those shown in FIG. 12. Where the anchor transcription factor is ppar-gamma (PPARG), suitable ligands include, but are not limited to, those shown in FIG. 13. Where the anchor transcription factor is in the E2F family, an example of a suitable ligand is shown in FIG. 19, which ligand may function as an anchor to recruit a repressor or may also function in other embodiments as a means of recruiting an activator to the promoter of a cell death gene, e.g., as illustrated in FIG. 3.

In addition to the anchor transcription factor ligand, the CIPs employed in these embodiments also include a ligand for an oncogenic transcription factor. This embodiment is of particular significance in treatment of cancer, where the TF-CIP causes the cancer cell to kill itself with its own driver. Oncogenic transcription factors are transcription factors whose activity contributes to a neoplastic, e.g., cancerous, disease condition. The oncogenic transcription factor may vary, e.g., depending on the particular nature of the disease condition being treated, where examples of oncogenic transcription factors include, but are not limited to: hormonal receptors (e.g., estrogen, androgen and progesterone receptors and the like), oncogene drivers (e.g., MYC, MLL fusion proteins, ETS fusion proteins, SS18-SSX fusion proteins and the like), translocated fusion oncogenes and proteins that regulate cell cycle entry (e.g., E2F family members and the like), etc. FIG. 14 provides examples of oncogenic transcription factors that may be targeted by TF-CIPs in embodiments of the invention (PMID: 33634124).

Ligands for exemplary cancer drivers or modulators are illustrated in FIGS. 16 to 19, and include ligands for the ERG fusion and the Fli-fusiion oncogenes, FEV transcriptional activator and Myc, which drives proliferation due to oncogenic mutations in many growth factor receptors and their associated signal molecules.

In some instances, the oncogenic transcription factor is a hormonal receptor. Hormonal receptors that may be employed as the oncogenic transcription factor in embodiments of the invention include, but are not limited to: estrogen receptor (ER), e.g., such as those provided in FIG. 20, androgen receptor (AR), e.g., such as those provided in FIG. 21, progesterone receptor (PR), e.g., such as those provided in FIG. 22 and the like. Examples of TFCIPs that use ligands for the estrogen receptor and the androgen receptor are shown in FIG. 4 and their effects on cancer cells in FIG. 6. Any convenient ligands for these hormonal receptors may be employed, where suitable ligands include small molecule ligands that are capable of specifically binding to the target hormonal receptor without any relevant negative impact on the hormonal receptor's ability to enhance transcription of the target proapoptotic gene when complexed with the anchor transcription factor by a CIP, i.e., the transcription-activating activity of the oncogenic transcription factor. The molecular weight of these ligands may vary, and in some instances ranges from 150 Daltons to 500 Daltons such as 250 Daltons to 400 Daltons. Suitable ligands for the anchor transcription factor include BCL6 inhibitors, such as BI3812. Others include FOXO3A (FIGS. 11 and 12) which bind and activate the expression of a number of pro-apoptotic genes in breast and other cancers (PMID 15084260). Others may be chosen using any convenient protocol, such as in silico screening protocols, and the like, such as described in detail in FIG. 8.

In some instances, the oncogenic transcription factor is BAF53a (a.k.a. ACTL6a). BAF53a is a subunit of the BAF or mSWI/SNF chromatin regulatory complex (PMID: 9845365), which opposes polycomb mediated repression over the genome (PMID: 27941796) and plays prominent roles in activating developmentally repressed genes (PMID: 20110991). In these cancers, BAF53a drives both initiation and proliferation and is expressed highly and specifically. In a specific embodiment of this invention TF-CIPs can be applied in Squamous Cell Cancer (SCC). Thus, using the overexpressed BAF53a to kill the SCC is a specific treatment for SCC. Using the systematic approach defined in FIG. 8, BAF53a ligands were identified that are illustrated in FIG. 23. For TF-CIPs, these BAF53a ligands may be attached chemically to convenient linkers, e.g., as described above, and these in turn attached chemically to a ligand for a TF that binds the promoter of a proapoptotic gene, such as FOXO3A or that inhibit an anti-apoptotic gene such as inhibitors of BCL6 and others, such as those described above. The resulting TF-CIP may be directed at specially killing the SCC and not normal cells because the normal cells do not have amplification of BAF53a.

The first and second ligands of the CIPs employed in embodiments of the above methods may be linked to each other by any convenient linker. As reviewed above, linkers of interest are linkers that provide for a stable association of the first and second ligands in a manner such that the first and second ligands are capable of specifically binding to their respective endogenous factors in the cell. As the linker provides for stably associating the first and second ligands with each other, the first and second ligands do not dissociate from each other under cellular conditions, e.g., conditions at the surface of a cell, conditions inside of a cell, etc. Linkers may be provided for stable association of the first and second ligands using any convenient binding, such as covalent or non-covalent binding, where in some instances the linker component is covalently bound to both the first and second ligands. In some embodiments, the linker may be an alkyl chain, an alkoxy chain, an alkyenyl chain or a alkynyl chain, where the number of carbon atoms in the chain may vary, ranging in some instances from 2 to 25, such as 5 to 20, where one or more carbon atoms are replaced with NH or CH₃—N to provide covalent bonding to the first and second ligands. Linkers may be bound to the first and second ligands at positions that do not negatively impact the ability of the ligands to bind to their respective endogenous factors. In some embodiments there will be no discrete linker, but rather a linking component that is a molecule which induces proximity of the targeted transcription factor to the anchoring transcription factor on the promoter of the target gene. Examples of such linking components include molecular glues, in which the two different sides of the singular molecule will each bind a separate transcription factor, e.g., as illustrated in FIG. 2.

Methods of enhancing transcription of pro-apoptotic genes finds use in, for example, treatment of oncogenic transcription factor mediated neoplastic disease conditions, e.g., cancer. Cancers which may be treated using embodiments of the invention include oncogenic receptor (e.g., hormonal receptor, such as estrogen, progesterone and androgen receptor) mediated cancers, oncogenic driver (e.g., myc) mediated cancers, translocated fusion oncogene (e.g., those shown in FIG. 15) mediated cancers, cell cycle entry transcription factor mediated cancers, etc. Specific cancers of interest that may be treated according to embodiments of the invention include, but are not limited to: Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, etc.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer,

Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sezary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like. Cancers that may be treated further include, epithelial cancers, such as carcinomas, such as acinar carcinoma , acinic cell carcinoma, acinous carcinoma, adenocystic carcinoma, adenoid cystic carcinoma, adenosquamous carcinoma, adnexal carcinoma, adrenocortical carcinoma, alveolar carcinoma, ameloblastic carcinoma, apocrine carcinoma, basal cell carcinoma, bronchioloalveolar carcinoma, bronchogenic carcinoma, cholangiocellular carcinoma, chorionic carcinoma, clear cell carcinoma, colloid carcinoma, cribriform carcinoma, ductal carcinoma in situ, embryonal carcinoma, carcinoma en cuirasse, endometrioid carcinoma, epidermoid carcinoma, carcinoma ex mixed tumor, carcinoma ex pleomorphic adenoma, follicular carcinoma of thyroid gland, hepatocellular carcinoma, carcinoma in situ, intraductal carcinoma, Hürthle cell carcinoma, inflammatory carcinoma of the breast, large cell carcinoma, invasive lobular carcinoma, lobular carcinoma, lobular carcinoma in situ (LCIS), medullary carcinoma, meningeal carcinoma, Merkel cell carcinoma, mucinous carcinoma, mucoepidermoid carcinoma, nasopharyngeal carcinoma, non-small cell carcinoma , non-small cell lung carcinoma (NSCLC), oat cell carcinoma, papillary carcinoma, renal cell carcinoma, scirrhous carcinoma, sebaceous carcinoma, carcinoma simplex, signet-ring cell carcinoma, small cell carcinoma , small cell lung carcinoma, spindle cell carcinoma, squamous cell carcinoma, terminal duct carcinoma, transitional cell carcinoma, tubular carcinoma, verrucous carcinoma, and the like.

Embodiments of the methods may include assessing whether a subject suffering from a neoplastic disease has a particular type of cancer. For example, where a subject has breast cancer, the methods may include assessing whether the breast cancer is ER and/or PR positive, and then employing an appropriate TFCIP to treat the particular cancer. For example, if the breast cancer is ER positive, a CIP having a ligand that binds to ERα may be employed. Another example is prostatic cancer driven by the translocated ETS family members to a gene that drives high level expression of the ETS fusion protein. Here the ETS fusion protein can be hijacked by, for example, an ERG binding ligand, e.g., selected as described in FIG. 8 or taken from known ERG binding molecules (e.g., as shown in FIG. 17), linked to the BCL6 inhibitor BI3812 or others (e.g., as discussed above) similar to the strategy used to make the ER-TF-CIPS shown in FIGS. 3 and 4 and that show killing activity shown in FIG. 6. Another example is in prostatic cancer driven by the over-expressed androgen receptor or its regulatory regions (PMID:30033370). Here an androgen binding moiety can be chemically linked to an inhibitory ligand for the BCL6 anti-apoptotic protein as shown in FIG. 4. The AR-TF-CIP induces proximity of the activating AR to the BCL6 protein bound to the promoters of genes that activate cell death (e.g., Table 1, above).

### Reducing Expression of Over-Expressed Genes, e.g., Oncogenes, Trisoimic Genes and Amplified Genes

Embodiments of the invention include methods of reducing transcription of a specific gene whose overexpression contributes to a pathologic process. Examples include, but are not limited to, oncogenes that are pathogenic due to amplication of their DNA (for example BAF53a in squamous cell carcinoma, genes that are overexpressed as a result of trisomy (for example Down Syndrome), genes that are overexpressed for a variety of reasons and that lead to a disease state (for example Tumor Necrosis Factor in arthritis); and alleles containing triplet repeats. The general approach is illustrated in FIG. 1, described above. By reducing transcription of a gene, e.g., oncogene, is meant limiting or repressing, e.g., inhibiting, transcription of the gene, e.g., oncogene. Aspects of embodiments of these methods include providing in the cell a chemical inducer of proximity (CIP) which links a first endogenous anchor transcription factor that binds to a promoter of the target gene, e.g., oncogene, and a second endogenous transcription modulating factor, wherein CIP mediated linkage of anchor transcription factor and transcription modulating factor reduces transcription of the target gene, e.g., oncogene, in the cell. The magnitude of decrease in transcription may vary. In some instances, the magnitude of decrease may be 2-fold or more, such as 5-fold or more, including 10-fold or more. In some instances, CIPs employed in these embodiments are generally as described above and include a first ligand that specifically binds to the anchor transcription factor and a second ligand that specifically binds to the transcription modulatory factor, where these first and second ligands are joined by a bond or suitable linker, e.g., as described above. Where the target gene is an oncogene, the target oncogene in such instances may vary. Examples of oncogenes the transcription of which may be reduced include, but are not limited to: HER-2/neu, RAS, MYC, SRC, hTERT, antiapoptotic proteins such as BCL-2, Ret, PI3Kinase, BRAF, EGFR, CTNNB1 and the like. Additional oncogenes that may be targeted include, but are not limited to, those described in Bailey et al, “Comprehensive Characterization of Cancer Driver Genes and Mutations,” Cell. 2018 Aug. 9; 174(4):1034-1035. doi: 10.1016/j.ce11.2018.07.034. The anchor transcription factor that is employed in these embodiments will be one that binds to a transcription factor-binding site or response element of the target oncogene. As such, an anchor transcription factor may vary depending on the target oncogene. Transcription factors promoting the expression of these oncogenes that may be employed in embodiments of the invention include, but are not limited to, those described in: PMID: 32728250, PMID: 32728217 and PMID: 32814038. Any convenient ligands for these anchor transcription factors may be employed, where suitable ligands include small molecule ligands that are capable of specifically binding to the target anchor transcription factor without any relevant negative impact on the anchor transcription factor's ability to bind to target DNA binding site. The molecular weight of these ligands may vary, and in some instances ranges from 200 to 1200 Daltons such as 300 to 500 Daltons. Suitable ligands for the anchor transcription factor may be chosen using any convenient protocol, such as in small molecule binding screens or silico screening protocols. Ligands suitable for use with E2F and Myc include, but are not limited to, those described FIGS. 18 and 19.

In addition to the anchor transcription factor ligand, the CIPs employed in these embodiments may also include a ligand for a transcription modulating factor that reduces transcription of the target gene, e.g., oncogene, in the cell. Transcription modulatory factors that reduce transcription of the oncogene in the cell when complexed with the anchor transcription factor via the CIP may vary, and include transcriptional repressors. Examples of transcriptional repressors include, but are not limited to, heterochromatin protein 1 (HP1) repressor proteins, KRAB repressor proteins, H3K9 methyltransferases, histone deacetylases etc. Any convenient ligands for these transcription modulatory factors may be employed, where suitable ligands include small molecule ligands that are capable of specifically binding to the target transcriptional modulatory factor without any relevant negative impact on the factor's ability to reduce transcription of the target oncogene when complexed with the anchor transcription factor by a CIP. The molecular weight of these ligands may vary, and in some instances ranges from 75 to 1000 such as 200 to 400 Daltons. Examples of such ligands include both agonists and antagonists. Suitable ligands for the anchor transcription factor may be chosen using any convenient protocol, such as in silico screening protocols, and the like, such as described below.

The first and second ligands of the CIPs employed in embodiments of the above methods may be linked to each other by any convenient linker component. As reviewed above, linker components of interest such as those described above provide for a stable association of the first and second ligands in a manner such that the first and second ligands are capable of specifically binding to their respective endogenous factors in the cell. As the linker component provides for stably associating the first and second ligands with each other, the first and second ligands do not dissociate from each other under cellular conditions, e.g., conditions at the surface of a cell, conditions inside of a cell, etc. Linker components may be provided for stable association of the first and second ligands using any convenient binding, such as covalent or non-covalent binding, where in some instances the linker component is covalently bound to both the first and second ligands. In some embodiments, the linker may be an alkyl chain, an alkoxy chain, an alkyenyl chain or a alkynyl chain, where the number of carbon atoms in the chain may vary, ranging in some instances from 2 to 25, such as 5 to 20, where one or more carbon atoms are replaced with NH or CHs-N. Linkers may be bound to the first and second ligands at positions that do not negatively impact the ability of the ligands to bind to their respective endogenous factors. In other cases, the first and second ligand are contained within one two-sided molecule or molecular glue. Examples of molecular glues include FK506, rapamycin, and cyclosporin A, etc.

Methods of reducing transcription of oncogenes finds use in, for example, treatment of oncogene mediated neoplastic disease conditions, e.g., cancer, where examples of such cancers include, but are not limited to, those described above.

Embodiments of the invention also include reducing transcription of mutant extended nucleotide repeat (NR) containing alleles. For example, where NR containing genes are mono allelic, embodiments of the methods may include suppressing expression of the NR containing monoallele by employing an anchor transcription factor that binds to the allele containing the NR. In such embodiments, the target gene is a gene that includes a mutant extended NR, such as a TNR, where the mutant extended nucleotide repeat domain is not present in normal versions of the gene. By mutant extended nucleotide repeat (NR) is meant a domain (i.e., region) of the gene that includes multiple adjacent repeats of units of 2 or more nucleotides, where a given repeating unit of nucleotides may vary in length, ranging in some instances from 2 to 10 nucleotides, such as 3 to 6 nucleotides, where examples of repeat unit lengths include units of 2 nucleotides (e.g., where the mutant extended nucleotide repeat is a dinucleotide repeat), 3 nucleotides (e.g., where the mutant extended nucleotide repeat is a trinucleotide repeat), 4 nucleotides (e.g., where the mutant extended nucleotide repeat is a tetranucleotide repeat), 5 nucleotides (e.g., where the mutant extended nucleotide repeat is a pentanucleotide repeat) or 6 nucleotides (e.g., where the mutant extended nucleotide repeat is a hexanucleotide repeat). Within a given domain, the domain may be homogeneous or heterogeneous with respect to the nature of the repeat units that make up the domain. For example, a given domain may be made up of a single type of repeat unit, i.e., al the repeat units of the domain share the same (i.e., identical) sequence of nucleotides, such that it is a homogenous mutant NR domain, Alternatively, a given domain may be made up of two or more different types of repeat units, i.e., repeat units that have differing sequences, such that it is a heterogeneous mutant NR domain. The mutant extended nucleotide repeat domain may be present in a coding or non-coding region of the target gene. In some instances, the extended nucleotide repeat domain is present in a coding region of the target gene. In some instances, the extended nucleotide repeat domain is present in a non-coding region of the target gene. The length and particular sequence of the mutant extended nucleotide repeat may vary.

In some instances, the mutant extended nucleotide repeat is a mutant extended trinucleotide repeat. By mutant extended trinucleotide repeat is meant a domain (i.e., region) of the gene that includes multiple adjacent repeats of the same three nucleotides, where the length and particular sequence of the mutant extended trinucleotide repeat may vary and the mutant extended trinucleotide repeat domain is not present in normal versions of the gene. The extended trinucleotide repeat domain may be present in a coding or non-coding region of the target gene. In some instances, the extended trinucleotide repeat domain is present in a coding region of the target gene. In some instances, the extended trinucleotide repeat domain is present in a non-coding region of the target gene. In embodiments, the mutant repeat domain is present in a non-coding region of the target gene, such as the CTG expansion located in the 3′ untranslated region of the dystrophia myotonica-protein kinase gene, which leads to Myotonic dystrophy (DM), In some instances, the mutant repeat domain is present in a coding region of the target gene, such that in some instances its presence in the target gene results in a corresponding domain or region (e.g., polyQ domain) in a product encoded by the gene. In some instances of the method, the mutant extended TNN domain is a CTG repeat domain. In certain cases, the mutant extended trinucleotide repeat domain includes 26 or more CTG repeats (e.g,, 30 or more, 35 or more, etc.).

The mutant extended trinucleotide repeat may vary in terms of nucleotide composition and length. Specific trinucleotides of interest include, but are not limited to: CAG, CTG, CGG, CCC, GAA, and the like. In some instances, the mutant extended trinucleotide repeat domain is a CAG repeat domain. The particular length of the repeat domain (e.g., CAG repeat domain) may vary with the respect to the specific target gene so long as it results in deleterious activity, and in some instances is 25 repeats or longer, such as 26 repeats or longer. 30 repeats or longer, including 35 repeats or longer, 40 repeats or longer, 50 repeats or longer or even 60 repeats or longer. Specific target genes and expressed proteins of interest, diseases associated therewith and the specific length of repeat sequences of extended CAG repeats of interest, include (but are not limited to) those provided in the table, below.

| | | disease name/ | Pathogenic |
|---|---|---|---|
| Disease | | protein product | repeat length |
| Spinocerebellar | SCA1 | SCA1/ataxin 1 | 40~82 |
| ataxia type 1 | | | |
| Spinocerebellar | SCA2 | SCA2/ataxin 2 | 32~200 |
| ataxia type 2 | | | |
| Spinocerebellar | SCA3(MJD) | SCA3/ataxin 3 | 61~84 |
| ataxia type 3 | | | |
| Spinocerebellar | SCA7 | SCA7/ataxin 7 | 37~306 |
| ataxia type 7 | | | |
| Spinocerebellar | SCA17 | SCA17/TBP | 47~63 |
| ataxia type 17 | | | |
| Dentatorubral | DRPLA | DRPLA/atrophin 1 | 49~88 |
| pallidoluysian | | | |
| atrophy | | | |
| Spinal and bular | SBMA | Kennedy's | 38~62 |
| muscular atrophy | | disease/androgen | |
| | | receptor protein | |
| Huntington's | HD | Huntington's | 40~121 |
| disease | | Disease/huntingtin | |
| | | protein | |

The pathogenic repeat lengths shown are approximate and represent the most common range of pathogenic repeat lengths. The lower of the two numbers shown for each pathogenic repeat length indicates the length at which pathogenic effects of the expansion begin to occur, Although both cellular copies of autosomal genes responsible for NR diseases may contain NR domains, commonly one copy of the targeted gene is mutated to have an expanded NR segment, whereas the other copy (i.e., allele) contains a unexpanded

### Enhancing Transcription of Therapeutic Beneficial Genes

Embodiments of the invention include methods of enhancing transcription of a therapeutically beneficial gene in a cell, e.g., as illustrated in general in FIG. 1. By enhancing transcription of a therapeutically beneficial gene is meant increasing transcription of the therapeutically beneficial gene. The magnitude of increase in transcription may vary. In those instances where transcription of the therapeutically beneficial gene is not detectable by a suitable assay, embodiments of the methods result in an enhancement of transcription so that transcription is detectable, e.g., by detecting the expression product of the therapeutically beneficial gene or activity thereof, e.g., improvement in one or more symptoms of a disease condition associated with a deficit of the expression product of the therapeutically beneficial gene. In those instances where there is a base level of transcription that is detectable, the magnitude of increase may vary and, in some instances, may be 2-fold or more, such as 5-fold or more, including 10-fold or more.

The methods may result in enhancing transcription of a variety of different therapeutically beneficial genes that may or may not be haploinsufficient genes. Therapeutically beneficial genes are genes the expression products of which are beneficial with respect to a given disease condition. Therapeutically beneficial genes may be genes in which an increase in the amount of expression product thereof results in the improvement in one or more symptoms of a disease condition associated with a low amount, e.g., an amount below that of a normal control, of expression product of that gene. As illustrated in FIG. 25, treatment of a disease produced by loss of function mutations in one allele of a dosage-dependent gene may be treated with TF-CIPs according to embodiments of the invention, which may be specific for each dosage dependent gene. Specific therapeutically beneficial genes of interest for which transcription may be enhanced in embodiments of the invention include, but are not limited to: genes encoding rate-limiting enzymes, e.g., tryptophan hydroxylase (TPH2,for serotonin production), tyrosine hydroxylase (TH, for dopamine synthesis in Parkinson's disease), protein C, Protein S, Factor 8, 5′-Aminolevulinic acid synthase (ALA-S) in heme synthesis and the like; haploinsufficient genes, e.g., ARID1B (BAF250b), TBR1, CHD8; BCL11a; and other haploinsufficient genes. A curated list of haploinsufficient genes that may be targeted in embodiments of the invention can be found in Clinical Genome Resource https://search.clinicalgenome.org/kb/curations. Autism genes that produce social dysfunction by reduced expression and may be targeted in embodiments of the invention can be found in a list curated by the found in the Simons Foundation for Autism Research at: https://gene.sfari.org/database/human-gene/. Several of these genes have been shown to operate in adult neurons of the dorsal raphe (PMID:34239048; 32568072) indicating that the disease may be treated even in adults. This is supported by published studies showing that transient reversal of the social features of autism can be brought about with small molecules that modulate serotonin effects, but that are too toxic for actual use in humans (PMID 34239048)

Aspects of the methods of these embodiments include providing in the cell, e.g., via a protocol as described above, a chemical inducer of proximity (CIP) which links a first endogenous anchor transcription factor that binds to a promoter of the therapeutically beneficial gene and a second endogenous transcription modulatory factor, e.g., transcription factor, wherein CIP mediated linkage of anchor and transcription modulatory factors enhances transcription of the therapeutically beneficial gene in the cell. In some instances, CIPs employed in these embodiments are generally as described above and include a first ligand that specifically binds to the anchor transcription factor and a second ligand that specifically binds to the transcription modulatory factor, where these first and second ligands are joined by a bond or suitable linker, e.g., as described above.

A variety of different anchor transcription factors may be employed in methods of these embodiments, where anchor transcription factors may readily be chosen based on the specific therapeutically beneficial gene and transcription factors therefor. For example, where the beneficial therapeutic gene is TPH2, any transcription factor therefore may be employed as the anchor transcription factor, where examples of such transcription factors include, but are not limited to: FEV, EN1, GATA2, GATA3, LMX1 B, POU3F2, INSM1, ESR2, CTCF, NR3C1, and REST, etc. The selection of pairs of transcription factors selectively expressed in serotonergic cells of the dorsal raphe is illustrated in FIG. 7A. For TPH2 a transcription factor that may be employed is FEV, which binds to the promoter of the TPH2 gene (PMID: 10575032). Loss of FEV leads to reduced serotonin production (PMID: 12546819). Ligands for FEV were identified by virtual screening, and biochemical analysis as in FIG. 8 and are illustrated in FIG. 17. These ligands can be attached to linkers, such as described in FIGS. 24A and 24B, and to ligands for other transcription factors expressed selectively in the dorsal raphe of the brain to yield cell type specific and circuit-selective expression of serotonin production. Because TPH2 is rate limiting for serotonin synthesis this would produce an increase in serotonin production to treat, e.g., mood disorders, depression, autism and other serotonin-related diseases (FIG. 29, Panel A). The synthetic methods to produce TF-CIP's that recruit the activator BRD4 to the promoter of the TPH2 gene by binding to FEV are shown in FIGS. 27 and 28.

A parallel strategy may be used to increase dopamine synthesis by stimulating production of the rate-limiting enzyme, tyrosine hydroxlase, in the substantia nigra in early Parkinson's disease, where dopaminergic cells have not been entirely lost, as illustrated in FIG. 29, Panel B. The rationale for increasing dopamine synthesis in substantia nigra dopaminergic neurons is that these neurons stop producing dopamine in early stages of Parkinson's disease (PD) (Heo et al., 2020 PMID: 31928877). These “dormant” dopaminergic neurons are associated with PD symptoms prior to neuron death. Importantly, Heo et al. found that increasing the activity of substantia nigra dopamine neurons in PD rodent models rescued dopamine production and motor control. Their results suggest that restoring dopamine production in the substantia nigra of early-stage PD may rescue motor function and thereby serve as a therapeutic intervention for PD—for which none currently exist. As shown in FIG. 29, Panel B, a TF-CIP designed to increase transcription of the rate-limiting enzyme for dopamine synthesis, tyrosine hydroxylase (TH), can be used to enhance dopamine production.

As a general strategy to encode cell specificity into a TF-CIP, transcription factor pairs are targeted that are selectively co-expressed in the cell type of interest as illustrated in FIGS. 7A and 7C. Using dopaminergic neurons of the substantia nigra as an example, significantly co-expressed pairs of transcription factors across single human dopaminergic neurons dissected from the substantia nigra (Pearson correlation with r>0.5 and p<0.05 or overlap analysis p<0.05) (Agarwal et al., 2020 https://doi.org/10.1038/s41467-020-17876-0) were identified. The expression of dopaminergic transcription factor pairs in single cells across all human tissues (Human Protein Atlas; Karlsson et al., 2021 DOI: 10.1126/sciadv.abh2169) was then examined. Transcription factor pairs that showed co-expression outside of dopaminergic neurons were excluded, yielding 14 dopaminergic neuron-selective transcription factor pairs that can be used as targets for a TF-CIP to treat PD. These pairs are: RORA:SOX6, RORA:AEBP2, AEBP:LCORL, PBX1:SETBP1, PBX1:PIAS1, PBX1:ZEB1, FOXP2:ZEB1, FOXP2:KLF12, MYT1L:ZNF91, ZFHX3:ZNF91, ZFHX3:ZNF420, ZNF91:ZNF420, AFF3:THRB, and TAX1BP1:TCF25.

In another example, where the beneficial therapeutic gene is ARID1 B, the anchor transcription factor could be chosen using the systematic process described in FIG. 7B. Examples of such transcription factors include, but are not limited to: TBR1, OTX2, GATA2, GATA3, FEV, ETS1, KLF5, LMX1 B, PAX5,etc. TBR1 regulates the expression of ARID1b and TBR1 mutant mice have reduced production of ARID1B (PMID: 27325115 PMID: 25356899 PMID: 28584888). Ligands for these anchor transcription factors may be defined by the process illustrated in FIG. 8. The molecular weight of these ligands may vary, and in some instances ranges from 75 to 1000 Daltons such as 200 to 400 Daltons. Suitable ligands for the anchor transcription factor may be chosen or discovered using any convenient protocol, such as in silico screening protocols, as illustrated in FIG. 8.

In addition to the anchor transcription factor ligand, the CIPs employed in these embodiments also include a ligand for a transcription modulatory factor, e.g., which in some instances is a second endogenous transcription factor expressed in tissue type harboring the target cell, e.g., the dorsal raphe (for enhancement of TPH2 for serotonin production) or the human brain (for ARID1B). The transcription modulatory factor may vary, e.g., depending on the particular nature of the disease condition being treated and the cell in which transcription modulation is desired. For example, where the target beneficial therapeutic gene is TPH2, transcription modulatory factors that may be employed include, but are not limited to FEV, BRD4, P300/CBP, PAXS, POU6F2, KLF5, SOX14, POU3F3, SATB2, nBAF etc. In another example, where the target beneficial therapeutic gene is ARID1B, transcription modulatory factors that may be employed include, but are not limited to TBR1 and others chosen to optimize cell type specificity using the quantitative proteome map of the human body (PMID 32916130) and the procedures described in FIGS. 7B and 8.

Any convenient ligands for these transcription factors may be employed, where suitable ligands include small molecule ligands that are capable of specifically binding to the target transcription factor without any relevant negative impact on the transcription factor's ability to enhance transcription of the target beneficial therapeutic gene when complexed with the anchor transcription factor by a CIP, i.e., the transcription-activating activity of the transcription factor. A systematic process for selecting the ligand is illustrated in FIG. 8. The molecular weight of these ligands may vary, and in some instances ranges from 70 to 1100 Daltons such as 300 to 600 Daltons. Examples of such ligands include both agonists and antagonists. The first and second ligands of the CIPs employed in embodiments of the above methods may be linked to each other by any convenient linker, e.g., as described above. As reviewed above, linkers of interest are linkers that provide for a stable association of the first and second ligands in a manner such that the first and second ligands are capable of specifically binding to their respective endogenous factors in the cell. As the linker provides for stably associating the first and second ligands with each other, the first and second ligands do not dissociate from each other under cellular conditions, e.g., conditions at the surface of a cell, conditions inside of a cell, etc. Linkers may be provided for stable association of the first and second ligands using any convenient binding, such as covalent or non-covalent binding, where in some instances the linker component is covalently bound to both the first and second ligands. In some embodiments, e.g., as illustrated in FIGS. 24A and 24B, the linker may be an alkyl chain, an alkoxy chain, an alkyenyl chain or a alkynyl chain, where the number of carbon atoms in the chain may vary, ranging in some instances from 2 to 25, such as 5 to 20, where one or more carbon atoms are replaced with NH or CH₃—N. Linkers may be bound to the first and second ligands at positions that do not negatively impact the ability of the ligands to bind to their respective endogenous factors.

Methods of these embodiments find use in treating any disease condition for which increased transcription of a beneficial therapeutic gene is desired. Examples of such disease conditions include, but are not limited to: disease conditions associated abnormal expression of rate-limiting enzymes, e.g., TPH2 in disease conditions characterized by altered brain serotonin such as depression, anxiety, panic disorder, obsessive compulsive disorder, attention deficit hyperactivity disorder, sleep and circadian rhythm disorders, irritable bowel syndrome, PMS/hormone dysfunction, fibromyalgia, obesity, alcoholism, aggression, hyperserotonemia, social disorders, autism spectrum disorder, language disorders, etc. (PMIDs, 30552318, 32883965, 22826343, 22698760, 14675805). In addition, haploinsufficiency disease conditions, e.g., ARID1B causing intellectual disability, autism spectrum disorders, etc.; CHD7 gene causing CHARGE syndrome; RUNX2 gene causing Cleidocranial dysostosis; ADAMTS2, COL1A1, COL1A2, COL3A1, COL5A1, COL5A2, PLOD1, FKBP14, TNXB, COL12A1, B4GALT7, B3GALT6, CHST14, DSE, C1R, C1S, SLC39A13, ZNF469, PRDM5 causing Ehlers-Danlos syndromes; TNFAIP3 causing Haploinsufficiency of A20, FBN1 causing Marfan syndrome; SHANKS causing 22813 deletion syndrome, SCN1A causing Dravet syndrome, etc. A list of curated genes that produce human disease when the dosage is reduced by 50% and may be treated by embodiments of the invention is available from the Clinical Genome Resource at: https://search.clinicalgenome.org/kb/curations.

### Combination Therapy

Aspects of the present disclosure further include combination therapies. In certain embodiments, the subject method includes administering a therapeutically effective amount of one or more additional active agents in combination with a CIP of the invention. By combination therapy is meant that a CIP can be used in a combination with another therapeutic agent to treat a single disease or condition. Alternatively, a second TF-IP could be used to overcome drug-induced resistance to a first CIP or to boost the apoptotic activity of the first CIP. This could be accomplished by using a ligand to another anchoring transcription factor that binds to one or more of the apoptotic gene promoter/enhancers. In some embodiments, a CIP compound of the present disclosure is administered concurrently with the administration of another therapeutic agent, which can be administered as a component of a composition including the compound of the present disclosure or as a component of a different composition. In certain embodiments, a composition including a compound of the present disclosure is administered prior or after administration of another therapeutic agent. The subject compounds can be administered in combination with other therapeutic agents in a variety of therapeutic applications. Therapeutic applications of interest for combination therapy include those applications those described above.

As reviewed above, in some instances the CIPs are employed for treating neoplastic conditions. As such, the CIPs of such embodiments can be used jointly with any agent useful in the treatment of a neoplastic condition, such as anti-cancer agents and anti-tumor agents. Agents of interest which can be used jointly with the subject CIS compounds in such instances include, but are not limited to, Cancer chemotherapeutic agents, Agents that act to reduce cellular proliferation, Antimetabolite agents, Microtubule affecting agents, Hormone modulators and steroids, natural products and biological response modifiers, e.g., as described in greater detail below.

Cancer chemotherapeutic agents include non-peptidic (i.e., non-proteinaceous) compounds that reduce proliferation of cancer cells and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones. Peptidic compounds can also be used. Suitable cancer chemotherapeutic agents include dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. For example, dolastatin 10 or auristatin PE can be included in an antibody-drug conjugate of the present disclosure. Suitable cancer chemotherapeutic agents also include maytansinoids and active analogs and derivatives thereof (see, e.g., EP 1391213; and Liu et al (1996) Proc. Natl. Acad. Sci. USA 93:8618-8623);

duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD). Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide. Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine. Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like. Other anti-proliferative cytotoxic agents are navelbene, CPT-11, anastrazole, letrazole, capecitabine, reloxafine, cyclophosphamide, ifosamide, and droloxafine. Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, epothilone A, epothilone B, discodermolide; estramustine, nocodazole, and the like. Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation. Therefore, compounds that bind to the estrogen receptor are used to block this activity. Corticosteroids may inhibit T cell proliferation. Other suitable chemotherapeutic agents include metal complexes, e.g., cisplatin (cis-DDP), carboplatin, etc.; ureas, e.g., hydroxyurea; and hydrazines, e.g., N-methylhydrazine; epidophyllotoxin; a topoisomerase inhibitor; procarbazine; mitoxantrone; leucovorin; tegafur; etc. Other anti-proliferative agents of interest include immunosuppressants, e.g., mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc. Taxanes are suitable for use. “Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis). Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose). Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701. Biological response modifiers suitable for use include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ; (8) colony-stimulating factors; and (9) inhibitors of angiogenesis.

In some instances, the CIPs of the invention are employed in combination with immunotherapy agents. Examples of immunotherapy include anti-PD-1/PD-L1 immunotherapies, such as anti-PD-1/PD-L1 therapeutic antagonists, where such antagonists include but are not limited to e.g., OPDIVO® (nivolumab), KEYTRUDA® (pembrolizumab), Tecentriq™ (atezolizumab), durvalumab (MEDI4736), avelumab (MSB0010718C), BMS-936559 (MDX-1105), CA-170, BMS-202, BMS-8, BMS-37, BMS-242 and the like. Nivolumab (OPDIVO®) is a humanized IgG4 anti-PD-1 monoclonal antibody used to treat cancer. Pembrolizumab (KEYTRUDA®), formerly known as MK-3475, lambrolizumab, etc., is a humanized antibody used in cancer immunotherapy targeting the PD-1 receptor. Atezolizumab (Tecentriq™) is a fully humanized, engineered monoclonal antibody of IgG1 isotype against the PD-L1 protein. Durvalumab (MedImmune) is a therapeutic monoclonal antibody that targets PD-L1. Avelumab (also known as MSB00107180; Merck KGaA, Darmstadt, Germany & Pfizer) is a fully human monoclonal PD-L1 antibody of isotype IgG1. BMS-936559 (also known as MDX-1105; Bristol-Myers Squibb) is a blocking antibody that has been shown to bind to PD-L1 and prevent its binding to PD-1 (see e.g., U.S. NIH Clinical Trial No. NCT00729664). CA-170 (Curis, Inc.) is a small molecule PD-L1 antagonist. BMS-202, BMS-8, BMS-37, BMS-242 are small molecule PD-1/PD-L1 complex antagonists that bind PD-1 (see e.g., Kaz et al., (2016) Oncotarget 7(21); the disclosure of which is incorporated herein by reference in its entirety). Anti-PD-L1 antagonists, including e.g., antibodies, useful in the methods described herein include but are not limited to e.g., those described in U.S. Pat. Nos. 7,722,868; 7,794,710; 7,892,540; 7,943,743; 8,168,179; 8,217,149; 8,354,509; 8,383,796; 8,460,927; 8,552,154; 8,741,295; 8,747,833; 8,779,108; 8,952,136; 8,981,063; 9,045,545; 9,102,725; 9,109,034; 9,175,082; 9,212,224; 9,273,135 and 9,402,888; the disclosures of which are incorporated herein by reference in their entirety. Anti-PD-1 antagonists, including e.g., antibodies, useful in the methods described herein include but are not limited to e.g., those described in U.S. Pat. Nos. 6,808,710; 7,029,674; 7,101,550; 7,488,802; 7,521,051; 8,008,449; 8,088,905; 8,168,757; 8,460,886; 8,709,416; 8,951,518; 8,952,136; 8,993,731; 9,067,998; 9,084,776; 9,102,725; 9,102,727; 9,102,728; 9,109,034; 9,181,342; 9,205,148; 9,217,034; 9,220,776; 9,308,253; 9,358,289; 9,387,247 and 9,402,899; the disclosures of which are incorporated herein by reference in their entirety.

### Pharmaceutical Preparations

Also provided are pharmaceutical preparations of the CIP compounds. The CIP compounds can be incorporated into a variety of formulations for administration to a subject. More particularly, the CIP compounds of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. The formulations may be designed for administration via a number of different routes, including oral, buccal, rectal, parenteral, intraperitoneal, intradermal, transdermal, intracheal, intravenous, etc., administration. In pharmaceutical dosage forms, the compounds may be used alone or in appropriate association, as well as in combination, with other pharmaceutically active compounds. The following methods and excipients are merely exemplary and are in no way limiting.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethyl-cellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature. The compounds of this invention and their pharmaceutically acceptable salts which are active on topical administration can be formulated as transdermal compositions or transdermal delivery devices (“patches”). Such compositions include, for example, a backing, active compound reservoir, a control membrane, liner and contact adhesive. Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference in its entirety. Such patches may be constructed for continuous, pulsatile, or on demand delivery of pharmaceutical agents.

Optionally, the pharmaceutical composition may contain other pharmaceutically acceptable components, such a buffers, surfactants, antioxidants, viscosity modifying agents, preservatives and the like. Each of these components is well-known in the art. See, for example, U.S. Pat. No. 5,985,310, the disclosure of which is herein incorporated by reference. Other components suitable for use in the formulations of the present invention can be found in Remington's Pharmaceutical Sciences, Mace Publishing Company, Philadelphia, Pa., 17th ed. (1985). In an embodiment, the aqueous cyclodextrin solution further comprise dextrose, e.g., about 5% dextrose.

Dosage levels of the order of from about 0.01 mg to about 140 mg/kg of body weight per day are useful in representative embodiments, or alternatively about 0.5 mg to about 7 g per patient per day. For example, inflammation may be effectively treated by the administration of from about 0.01 to 50 mg of the compound per kilogram of body weight per day, or alternatively about 0.5 mg to about 3.5 g per patient per day. Those of skill will readily appreciate that dose levels can vary as a function of the specific compound, the severity of the symptoms and the susceptibility of the subject to side effects. Dosages for a given compound are readily determinable by those of skill in the art by a variety of means.

The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition. Dosage unit forms will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

It will be understood, however, that the specific dose level for any particular patient will depend upon a variety of factors including the age, body weight, general health, sex, diet, time of administration, route of administration, rate of excretion, drug combination and the severity of the particular disease undergoing therapy. As such, unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier. The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular peptidomimetic compound employed and the effect to be achieved, and the pharmacodynamics associated with each compound in the host.

### Kits & Systems

Also provided are kits and systems that find use in practicing embodiments of the methods, such as those described as described above. The term “system” as employed herein refers to a collection of two or more different active agents, present in a single composition or in disparate compositions, that are brought together for the purpose of practicing the subject methods. The term “kit” refers to a packaged active agent or agents. For example, kits and systems for practicing the subject methods may include one or more pharmaceutical formulations. As such, in certain embodiments the kits may include a single pharmaceutical composition, present as one or more unit dosages, where the composition may include one or more expression/activity inhibitor compounds. In yet other embodiments, the kits may include two or more separate pharmaceutical compositions, each containing a different active compound.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, portable flash drive, etc., on which the information has been recorded. Yet another means that may be present is a website address which may be used via the internet to access the information at a removed site. Any convenient means may be present in the kits.

The following examples are offered by way of illustration and not by way of limitation.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### I. CIP Compounds for Treatment of ER-Positive Breast Cancer by Hijacking Oncogenic Drivers

### A. Introduction

Breast cancer is responsible for over 40,000 deaths per year in the US. About 1 in 8 women will develop breast cancer and of these one in 39 will die of their cancer. If surgery is not effective in curing the cancer a number of treatments are used that include hormonal therapy, HER2 monoclonal antibodies and immunotherapy. Each of these is making a contribution to the effective treatment of breast cancer, yet many of the drugs used such as anthracyclines and others are non-specific in their antitumor actions and hence have broad toxicity that can be lethal. Therefore, the field has sought more specific routes for treatment. Here is described a new way of producing more specific death of the cancer cells relative to that of other cells and hence better treatments with fewer side effects.

### B. Overview

We have devised a general way of hijacking cancer-drivers to activate cell death pathways thereby killing any cancer cell that has a driving mutation. In the specific case of breast cancer, we hijack the estrogen receptor (ER) and/or the progesterone receptor (PR) to activate PUMA, BIM and other cell death genes. To do this we have invented small molecule chemical inducers of proximity (CIP) that have estrogen-like compounds linked at the C17 or C7 position to a ligand for a transcription factor(s) that normally binds to the regulatory regions of cell death genes, such as PUMA, BAX, BIM and BID and NOXA genes. Thus, estrogen, which normally drives the tumor instead activates a set of genes which results in programmed cell death. By this means the breast cancer pathways that drive proliferation are hijacked to specifically kill the cancer cell.

A large number of breast cancers are ER positive and PR positive. These hormonally responsive cancers are often treated using antagonists to these receptors and thereby reduce or slow the growth of the cancer. However, cancers are almost never successfully treated by agents that simply reduce the rate of growth of the cancer, because they simply recur when the treatment is stopped. Hence diverting the cancer's driving proliferative force to kill cancer cells is useful. We have invented a series of molecules (CIP) which chemically link estrogen-like molecules at C17 to a small linker and then to a ligand for transcription factors that control genes that induced apoptosis or programmed cell death such as the PUMA, BIM, BAX, BID and NOXA genes. These genes function downstream of p53 to initiate cell death when DNA damage occurs. In this way these genes suppress the formation of cancer and this is one of the major effectors of p53's tumor suppressor pathway. Many breast cancers have p53 inactivated and hence are unable to respond to DNA damage signals that might eliminate a cancer at its inception. In the following described embodiment, the transcription factor FOXO3a has been chosen, which regulates the PUMA, BIM and the BAX genes. The means for defining the anchor transcription factors are illustrated in FIG. 7. Although we describe the approach with BIM, we have conducted parallel experiments for the other ten pro-apoptotic genes.

A specific example of a TF-CIP that may be employed in such embodiments is estradiol linked to a small molecule binder of a transcription factor that activates the programmed cell death genes. Other embodiments of the invention use progesterone, any of several proapoptotic genes and any one of the following breast selective transcription factors: 1) FOXO3A, 2) PPARgamma, 3) HIF1A, 4) E2F1; 5) RUNX1 and MAZ, which bind to the PUMA as well as other the promoters for other killer genes, such as BIM. Normally estrogen will bind to the estrogen receptor and induce proliferation. In this embodiment of the present invention, a small molecule linking the estrogen receptor to a transcription factor (TF) which binds and coordinately activates killer genes is employed as a new therapy for ER positive breast cancer.

### C. Specificity of TF-CIP in Cancer

One of the many advantages of this approach of hijacking a cancer driver pathway to activate a cell death pathway is that it allows one to exploit the combinatorial specificity of the transcription factors to specifically activate the killer genes as well as the specificity of the cancer driver itself. This is illustrated generally in FIG. 7C and the methods detailed in FIGS. 7A and B The enhancement of specificity would then be:

(selectively of expression of TF-A)×(selectively of expression of the cancer driver)×(genomic specificity of TF-A)=selectivity of cell killing

There are many drugs and treatments used for treating human breast cancer including hormonal therapy, HER2 monoclonal antibodies and immunotherapy, each of which has a degree of specificity for the tumor. There are also a large number of drugs that are used in nearly all metastatic breast cancers that have little or no specificity for the cancer cell. Many of these merely stop the growth of the cancer cell as well as other cells.

The advantage of our approach is that it harnesses the cancer's own specific driving mechanism to activate a transcription factor that kills ER-positive breast cancer cells. An additional advantage of our approach is that it may be tailored to be very specific for the cancer cell. For example, if the estrogen receptor is expressed at a selective level of 6.5:1 in breast cancer cells and the transcription factor that bound the cell death gene were expressed at selective level of 10:1 the breast cancer cells would be killed with a 65-fold specificity thereby establishing an effective therapeutic window. The therapeutic window may be predicted as follows:

$$\frac{[ A 1 ]}{[ A 2 ]} \times \frac{[ B 1 ]}{[ B 2 ]} = Expected relative killing bewtween cell types 1 and 2 ;$$

[A1] is the concentration of the driver protein in cell type 1;
[A2] is the concentration of the driver protein in cell type 2;
   [B1] is the concentration of the anchoring TF in cell type 1; and
[B2] is the concentration of the anchoring TF in cell type 2

### D. Transcription Factor Identification

To identify transcription factors that could activate the expression of cell death genes we began by defining the cell death gene that was most effective at killing breast cancer cell lines. We examined each of the cell death genes shown in Table 1 using doxycycline inducible expression and found that several were effective, rapidly killing over 50% of the cells. To identify transcription factor pairs that may be used to selectively activate these genes only in target cells we used the procedures outlined In FIGS. 7A, 7B and 7C.

We then sought to identify transcription factors (TFs) with binding motifs within ±1 kb region around BIM TSS. This region has chromatin accessible to TFs binding as revealed by analysis of ATAC-seq data of two randomly selected patients from BRCA (Breast Invasive Carcinoma) cohort of TCGA (The Cancer Genome Atlas). Our method of transcription factor identification is described step-by-step in FIG. 7 and detected the presence of potential binding motifs for 337 TFs. The regulator regions of BIM, BAX and BID are also accessible in breast cancer cells and bind an overlapping group of TFs indicating that the proapoptotic genes function coordinately, which provides robust cell killing when a single TF is activated. Thus, several killer genes in breast cancer cells are vulnerable for transcriptional hijacking.

We analyzed expression of the selected 337 TFs in 5 cancer cell lines using publicly available RNA-seq data sets. The list of TFs exhibiting consistently high expression across cancer cell lines include YBX1, ATF4, HIF1A, MAZ, NFE2L2, TGIF1, SMAD2, TFDP1, MYC, DDIT3, STAT3, PNRC2, HES1, FOXO3A, RUNX1 and PPARG. Among these TFs, RUNX1, HIF1A, PAX9 and PPARG are highly specific to breast tissue (according to the Human Protein Atlas). HIF1A is master transcriptional regulator of adaptive response to hypoxia and plays important role in tumor angiogenesis and in hypoxia induced cell death.

### E. Development of Ligands for Anchoring Transcription Factors

Our method of developing ligands for anchoring TF's is described in a step-by-step approach in FIG. 8 and resulted in the identification of two classes of ligand for FOXO3A shown in FIGS. 10 and 11.

### F. Synthesis of Estrogen-Like ER Binder-Linker(n)-FOXO3a Binder

The TF-CIP, Estrogen-like ER binder-Linker(n)-FOXO3a binder illustrated below is synthesized as follows. The first component of our invention, which consist of three parts (A-L-B hijackers) is an estrogen-like molecule including, but not limited to, those shown in FIG. 20. These compounds have been selected on the basis of their ability to accommodate a linker at C17 and retain activity and ER binding. The C11 methoxy is employed in some of the molecules because of its reported favorable binding and estrogenic activity.

The second component the CIP, which consists of three parts (A-L-B hijackers) is a linker (L) of n carbon atoms designed to bridge the distance between the ER and the transcription factor (FOXO3a in this specific example). A suitable linker such as those described above is employed.

The third component of the CIP, which consists of three parts (A-L-B hijackers), is a small molecule that binds to the TF regulating the expression of the cell death genes: PUMA, BAX, BID and BIM. In FIG. 8 we provide a systematic, step-by-step approach to identifying ligands for the critical transcription factor, in this case FOXO3A. Ligands selected by this approach are shown in FIGS. 10 and 11. The above molecules are the product of the step-by-step process consisting of a structure-based virtual (in silico) screen using Schrodinger Glide of about 8 million flexible-ligand drug-like compounds from the ZINC library against a rigid crystal structure (PDB: 2uzk) of FOXO3a. The screen was conducted by picking a non-flexible pocket from the crystal structure of FOXO3a and using this pocket for the screen. Potentially toxic molecules and those predicted to have unfavorable pharmacologic characteristics were eliminated by manual curation as described in FIG. 8.

Molecules of the general structure A-L-B are tested for their ability to cause programmed cell death in estrogen-dependent cell lines such as MCF7, and MCF10 breast cancer cell lines. Although several hundred combinations of FOXO3A ligands (FIGS. 10 and 11), linkers (e.g., as described above) and estrogen analogues (FIG. 20) can be made and tested for their effectiveness, we show ten examples of a bifunctional molecule, below.

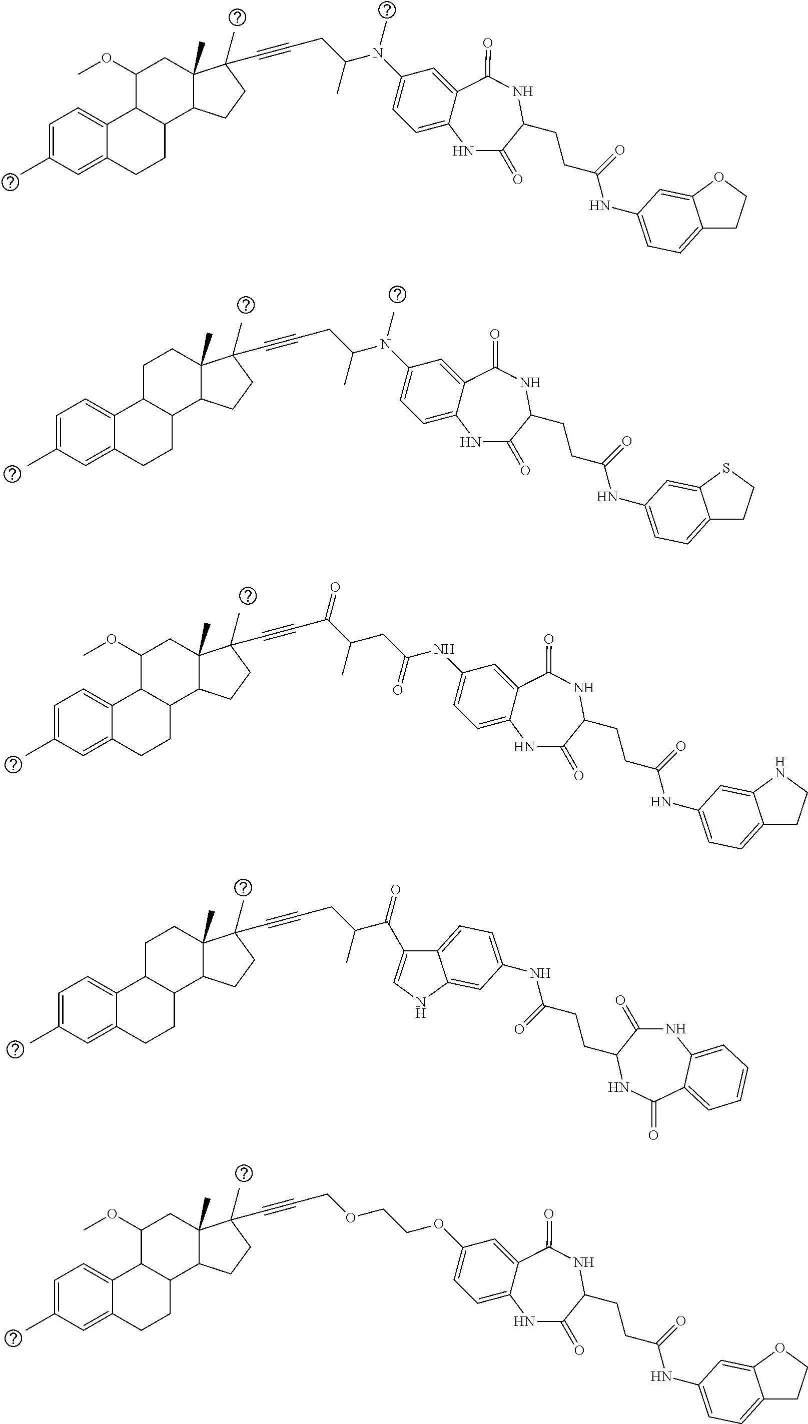

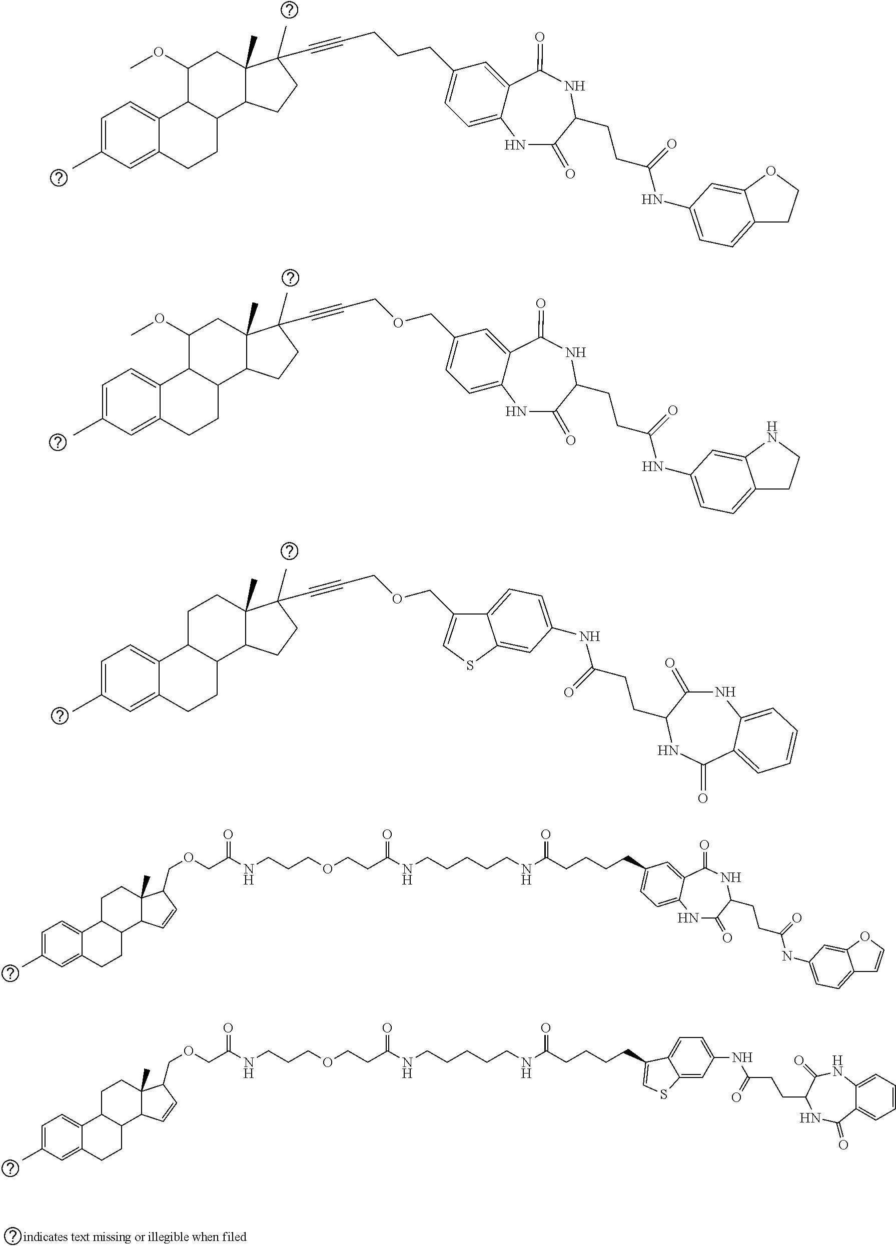

With the TF-CIPs illustrated in FIG. 4, we observe estrogen receptor-dependent cell killing meaning that the driving oncogenic pathway of these cells has been diverted to kill the cells as shown in FIG. 6. The cell killing is determined to be dependent upon induction of PUMA, BAX, BIM, NOXA and/or BID. In addition, the small molecule is tested in cancer cells that are not dependent on estrogen, such as the breast cancer cell line MDA-MB-231, the kidney cell line HEK293, and the lymphocyte cell line Jurkat, as well as others, for its ability to selective kill cells that are estrogen dependent. Because the estrogen receptor is expressed at about 6.5-fold higher levels in primary breast cancer cells, cancer killing is observed to be estrogen-dependent and relatively specific. The specificity of cell killing is compared to other agents used to treat breast cancer such as Adriomycin, TopoII inhibitors including etoposide and other anthracyclines, cyclophosphamide and others. This same approach is repeated for small molecules that consist of a synthetic estrogen linked to a small molecule that binds 1) MAZ; 2) PPARgamma; 3) HIF2; 4) RUNX1; 5) E2F1 and others illustrated in FIG. 8 that bind and can activate cell death genes including BIM, BAX, and BID.

### G. Anticipating the Development of Drug Resistance

We anticipate that resistance could arise for several reasons. For example, the binding site for the anchor TF could be mutated or the TF mutated to no longer serve as an anchor. In this case we use the method provided in FIG. 7 to pick a second anchor and the method described in FIG. 8 to define a ligand for the anchor. These ligands can then be used to construct another TF-CIP with estrogen or progesterone analogues (FIGS. 11 and 12) using established medicinal chemistry and used as a second line therapy, if resistance does develop. It is also possible that the estrogen receptor gene can be inactivated in response to treatment as a part of the selective process of tumor development. Since this could only happen if another driver appeared (for example one of the ones shown in FIG. 14) we would construct a TF-CIP to hijack the new driver.

Another example of using TF-CIPs as a way to cope with this possible complication, would involve constructing a TF-CIP to recruit a protein possessing a highly acidic domain to the PUMA TSS. Acidic domains are known for their ability to activate transcription. To identify acidic proteins highly specific to breast tumors, we used the following criteria: (1) Isoelectric point less than 5; (2) The protein is either nuclear or cytosolic (In later case it should have less than 500 amino acids to allow efficient transport to the nucleus); and (3) The average protein expression in tumor exceeds expression at normal tissue at least at 4 times. For this comparison we used gene expression databases consisting of 1085 samples from BRCA TCGA patients and 291 samples from healthy individuals. We found 28 proteins satisfying all 3 criteria: CENPF, CTXN1, DBNDD1, EPN3, ESRP1, FOXA1, GPRCSA, HN1, IF16, KRT8, LMNB1, MCM4, MUC1, PKIB, PRC1, PRR15, PYCR1, RACGAP1, S100P, SDC1, SLC9A3R1, SPP1, STARD10, TFF1, TNNT1, TPD52, UBE2S, ZWINT.

For the treatment of ER negative cancers, the progesterone receptor is targeted using an analogous group of molecules based on progesterone analogues that hijack killer genes.

### H. Synthetic Methods for ER-TF-CIPs

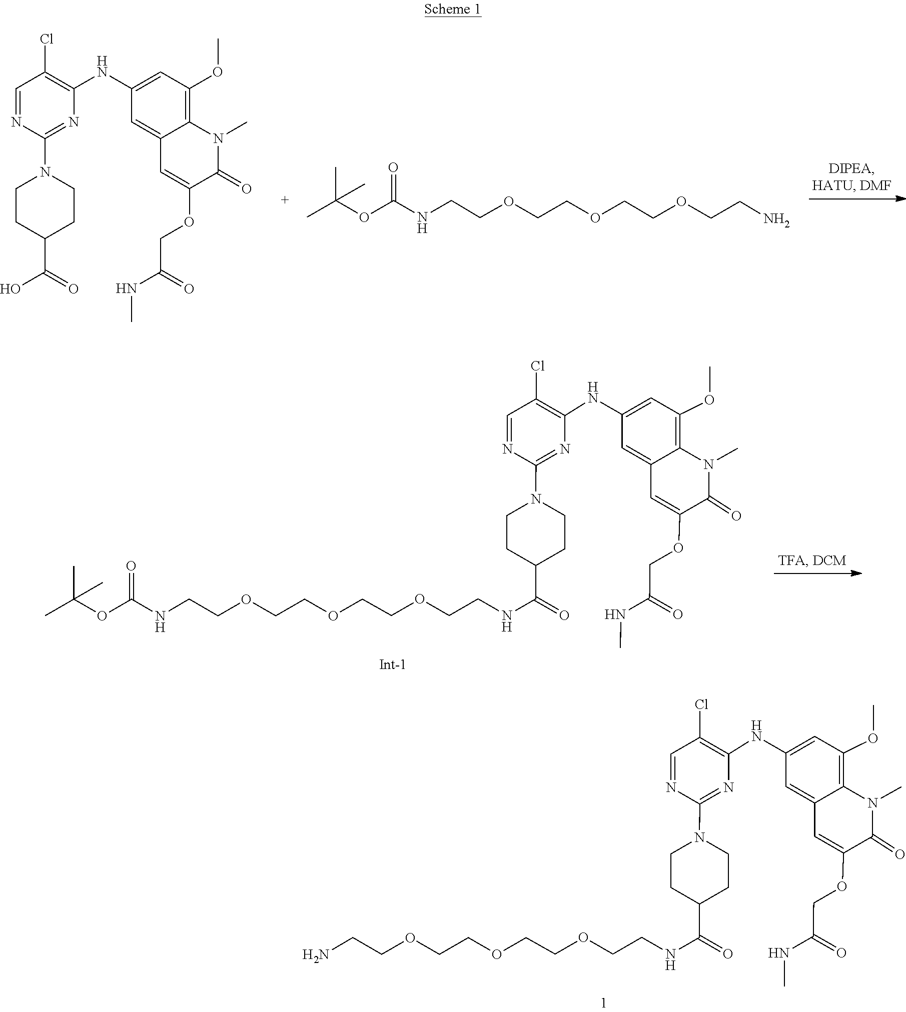 [SMILES: CNC(=O)COc1cc2cc(Nc3nc(N4CCC(C(=O)O)CC4)ncc3Cl)cc(OC)c2n(C)c1=O ; CC(C)(C)OC(=O)NCCOCCOCCOCCN ; CNC(=O)COc1cc2cc(Nc3nc(N4CCC(C(=O)NCCOCCOCCOCCNC(=O)OC(C)(C)C)CC4)ncc3Cl)cc(OC)c2n(C)c1=O ; CNC(=O)COc1cc2cc(Nc3nc(N4CCC(C(=O)NCCOCCOCCOCCN)CC4)ncc3Cl)cc(OC)c2n(C)c1=O]

### Step 1. Preparation of Int-1

A solution of 1-(5-chloro-4-((8-methoxy-1-methyl-3-(2-(methylamino)-2-oxoethoxy)-2-oxo-1,2-dihydroquinolin-6-yl)amino)pyrimidin-2-yl)piperidine-4- carboxylic acid (10 mg, 0.02 mmol, according to lit.1) t-Boc-N-amido-PEG3-amine and (30 mg, 0.1 mmol), HATU (21 mg, 0.05 mmol) and DIPEA (50 uL, 0.4 mmol) in DMF (0.25 mL) was stirred at room temperature for 1 h. The crude reaction was purified by HPLC to afford compound Int-1 (16 mg, 95%). MS obsd. [(M+H)+]:805.9

### Step 2. Preparation of Compound 1

A solution of Int-1 (16 mg, 0.02 mmol) was dissolved in DCM (1 mL) and added TFA 0.2 mL and stirred at room temperature for 0.5 h. The crude reaction was purified by HPLC to afford compound 1, (10 mg, 50%) as white solid. MS obsd. [(M+H)+]:705.8. ¹H NMR (500 MHz, DMSO) δ 8.88; (s, 1H), 8.08; (s, 1H), 7.99; (q, J=4.6 Hz, 1H), 7.89; (t, J=5.7 Hz, 1H), 7.78; (s, 3H), 7.58-7.51; (m, 2H), 7.01; (s, 1H), 4.56; (s, 2H), 4.51; (dt, J=13.2, 3.4 Hz, 2H), 3.80-3.97; (m, 6H), 3.61-3.51; (m, 9H), 3.40; (t, J=6.2 Hz, 2H), 3.20; (q, J=6.0 Hz, 2H), 3.03-2.86; (m, 4H), 2.66; (d, J=4.7 Hz, 3H), 2.42; (tt, J=11.6, 3.9 Hz, 1H), 1.71; (dd, J=13.4, 3.6 Hz, 2H), 1.50; (qd, J=12.4, 4.2 Hz, 2H).

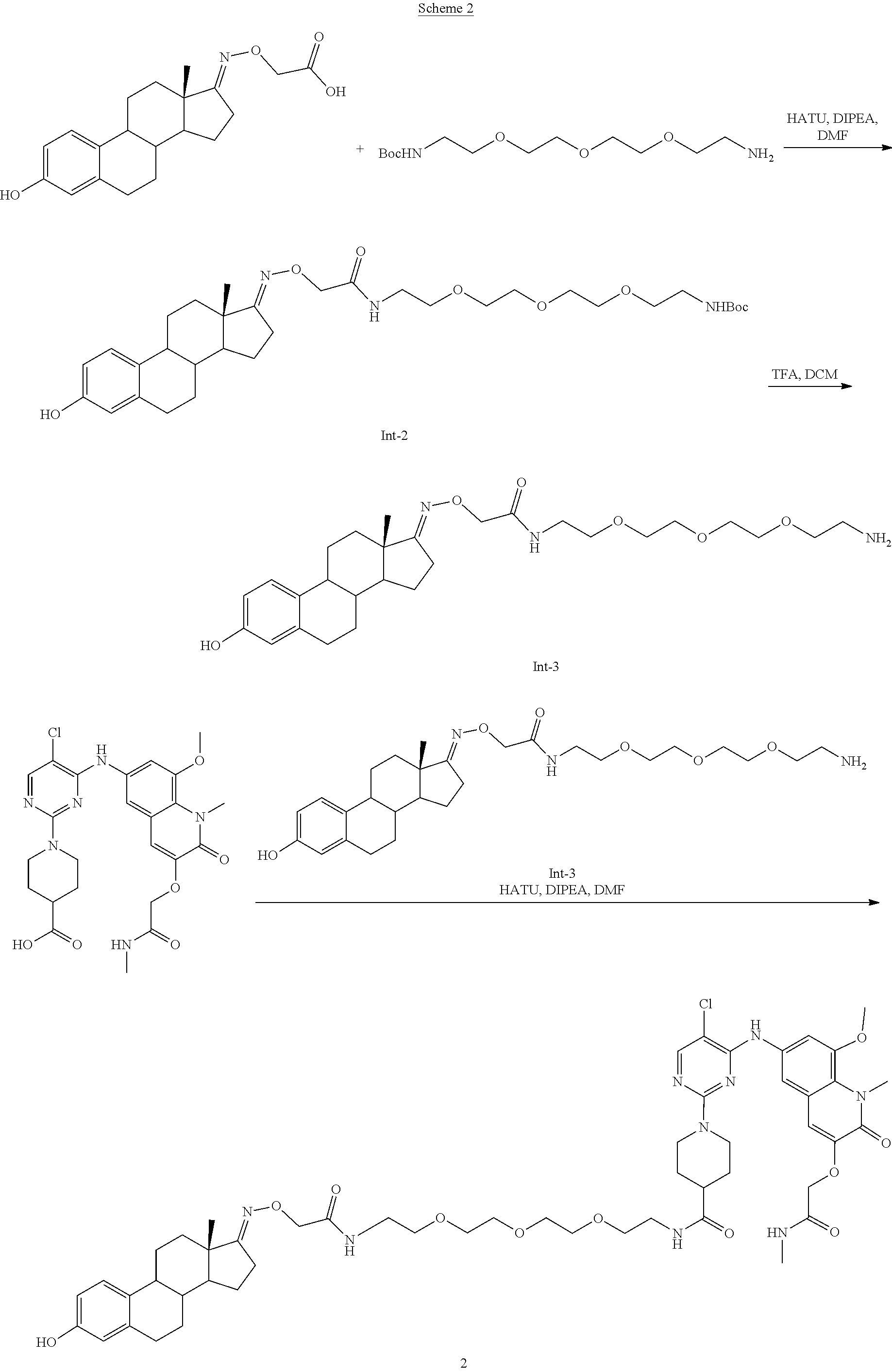

### Step 1. Preparation of Int-2

A solution of 2-((((13S,E)-3-hydroxy-13-methyl-6,7,8,9,11,12,13,14,15,16-decahydro-17H-cyclopenta[a]phenanthren-17-ylidene)amino)oxy)acetic acid (20 mg, 0.06 mmol, according to lit.1) t-Boc-N-amido-PEG3-amine and (17 mg, 0.06 mmol), HATU (33 mg, 0.09 mmol) and DIPEA (33 uL, 0.2 mmol) in DMF (0.5 mL) was stirred at room temperature for 1 h. The crude reaction was purified by flash chromatography to afford coupling product Int-2 (20 mg, 70%) as white solid

### Step 2. Preparation of Int-3

Int-3 (60 mg, 0.1 mmol) was dissolved in 1 mL DCM and subjected to 0.2 mL TFA at room temperature for 0.5 h. The crude reaction was purified by HPLC to afford compound Int-3 (30 mg, 60%). MS obsd. [(M+H)+]: 518.7

### Step 3. Preparation of Compound 2

A solution of Int-3 (30 mg, 0.06 mmol), and 1-(5-chloro-4-((8-methoxy-1-methyl-3-(2-(methylamino)-2-oxoethoxy)-2-oxo-1,2-dihydroquinolin-6-yl)amino)pyrimidin-2-yl)piperidine-4-carboxylic acid (30 mg, 0.06 mmol, prepared according to WO 2018/108704 A1), HATU (38 mg, 0.1 mmol) and DIPEA (50 uL, 0.3 mmol) in DMF (1 mL) was stirred at room temperature for 1 h. The crude reaction was purified by HPLC to afford compound 2, (10 mg, 50%) as white solid. MS obsd. [(M+H)+]:1031.1. ¹H NMR (500 MHz, DMSO) δ 9.07; (s, 1H), 9.03; (s, 2H), 8.15-8.08; (m, 1H), 7.94; (q, J=4.1 Hz, 1H), 7.86; (t, J=5.6 Hz, 1H), 7.52; (d, J=1.5 Hz, 2H), 7.41; (q, J=5.7 Hz, 1H), 7.07-6.99; (m, 2H), 6.51; (dq, J=8.2, 2.4 Hz, 1H), 6.44; (q, J=2.5 Hz, 1H), 4.56; (s, 2H), 4.47; (d, J=13.0 Hz, 2H), 4.34; (d, J=2.6 Hz, 2H), 4.02; (s, 1H), 3.91-3.84; (m, 5H), 3.45-3.60; (m, 8H), 3.43; (t, J=6.0 Hz, 2H), 3.39; (ddd, J=7.5, 4.7, 1.8 Hz, 2H), 3.31-3.24; (m, 2H), 3.19; (q, J=5.9 Hz, 2H), 2.97; (m, 3H), 2.74; (m, 1H), 2.65; (dd, J=4.6, 1.2 Hz, 3H), 2.54; (d, J=8.5 Hz, 2H), 2.28; (td, J=8.5, 4.1 Hz, 1H), 2.15; (q, J=4.4 Hz, 1H), 1.91-1.80; (m, 3H), 1.76-1.60; (m, 3H), 1.57-1.45; (m, 2H), 1.20-1.36; (m, 5H), 0.91-0.85; (m, 3H).

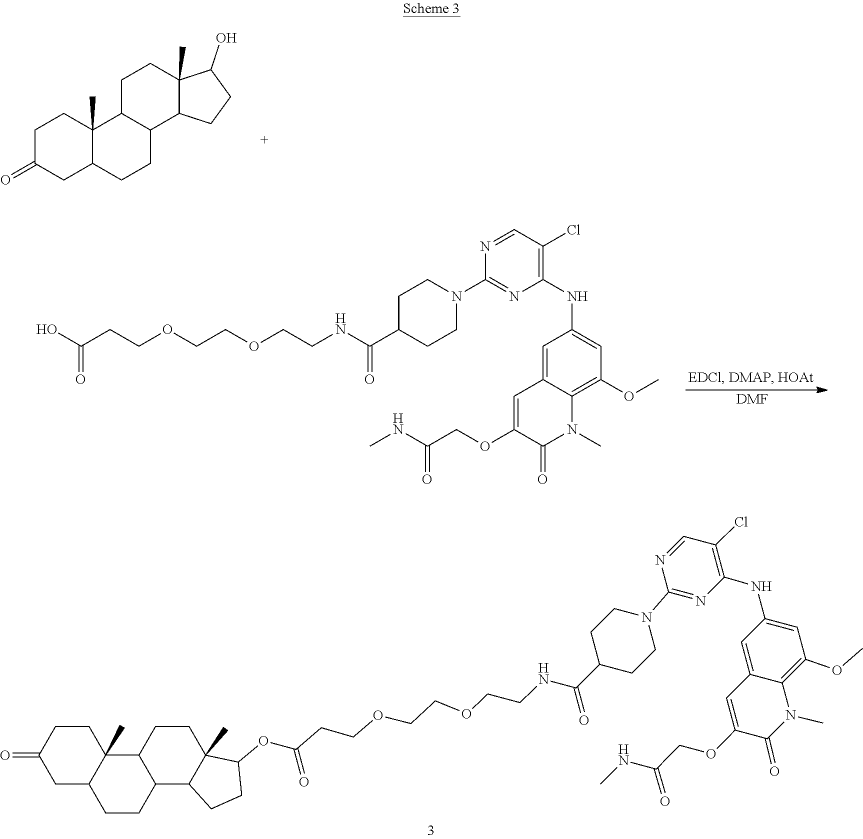

A solution of 3-(2-(2-(1-(5 -chloro-4-((8-methoxy-1-methyl-3-(2-(methylamino)-2-oxoethoxy)-2-oxo-1,2-dihydroquinolin-6-yl)amino)pyrimidin-2-yl)piperidine-4-carboxamido)ethoxy)ethoxy)propanoic acid (7 mg, 0.009 mmol), DHT (6 mg, 0.018 mmol), EDCI (6 mg, 0.04 mmol), DMAP (5 mg, 0.018), HOAt (5 mg, 0.009 mmol) in 0.25 mL DMF was stirred at room temperature for 1 h. The crude reaction was purified by HPLC to afford compound 3 (2 mg, 30%) as white solid. MS obsd. [(M+H)+]:963.1. ¹H NMR (500 MHz, DMSO) δ 10.84; (s, 1H), 8.82; (s, 1H), 8.06; (s, 1H), 7.98; (s, 1H), 7.84; (q, J=8.3 Hz, 2H), 7.45-7.60; (m, 2H), 7.00; (s, 1H), 4.55; (s, 2H), 4.53 — 4.45; (m, 3H), 3.87; (d, J=6.7 Hz, 4H), 3.61; (t, J=6.1 Hz, 4H), 3.52-3.47; (m, 9H), 3.24-3.15; (m, 5H), 3.00; (s, 2H), 2.91; (t, J=13.0 Hz, 4H), 2.78; (s, 3H), 2.78-2.75; (m, 4H), 2.66; (d, J=4.7 Hz, 3H), 2.53; (s, 6H), 2.43-1.99; (6H, m), 1.77-0.73; (22H, m).

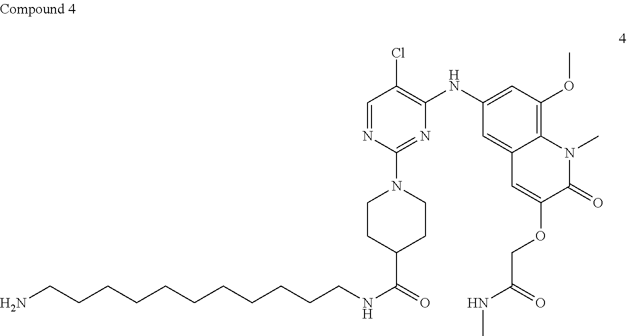 [SMILES: CNC(=O)COc1cc2cc(Nc3nc(N4CCC(C(=O)NCCCCCCCCCCCN)CC4)ncc3Cl)cc(OC)c2n(C)c1=O]

Compound 4 was prepared according to procedure of scheme 1.

MS obsd. [(M+H)+]: 699.8. ¹H NMR (500 MHz, DMSO) δ 8.76; (s, 1H), 8.00 (s, 1H), 7.92; (q, J=4.5 Hz, 1H), 7.70; (t, J=5.7 Hz, 1H), 7.59; (s, 1H), 7.51-7.44; (m, 2H), 6.93; (s, 1H), 4.50-4.41; (m, 4H), 3.80; (m, 5H), 2.94; (d, J=6.7 Hz, 2H), 2.80-2.90; (m, 2H), 2.60-2.69; (m, 3H), 2.58; (d, J=4.7 Hz, 3H), 2.35-2.26; (m, 2H), 1.62; (dd, J=13.4, 3.7 Hz, 2H), 1.43-1.17; (m, 20H).

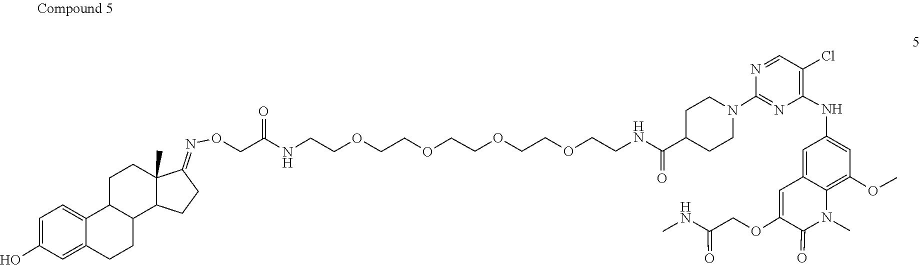 [SMILES: CNC(=O)COc1cc2cc(Nc3nc(N4CCC(C(=O)NCCOCCOCCOCCOCCNC(=O)CO/N=C5\CCC6C7CCc8cc(O)ccc8C7CC[C@]56C)CC4)ncc3Cl)cc(OC)c2n(C)c1=O]

Compound 5 was prepared according to procedure of scheme 2.

MS obsd. [(M+H)+]: 1075.2. ¹H NMR (500 MHz, DMSO) δ 8.93; (s, 1H), 8.81; (s, 1H), 8.00; (s, 1H), 7.89; (d, J=2.3 Hz, 1H), 7.79; (t, J=5.7 Hz, 1H), 7.46; (s, 2H), 7.33; (q, J=5.2 Hz, 1H), 6.90-6.95; (m, 2H), 6.43; (dd, J=8.4, 2.7 Hz, 1H), 6.36; (d, J=2.6 Hz, 1H), 4.48; (s, 2H), 4.42; (dd, J=10.5, 7.0 Hz, 2H), 4.26; (s, 2H), 3.79; (d, J=6.7 Hz, 4H), 3.40-3.50; (m, 12H) 3.20; (m, 2H), 3.11 (q, J=5.9 Hz, 3H), 2.83 (td, J=12.9, 2.8 Hz, 2H), 2.73-2.62; (m, 2H), 2.58; (d, J=4.7 Hz, 3H), 2.53-2.44; (m, 1H), 2.34; (m, 2H), 2.20-2.02; (m, 13H), 1.83-1.70; (m, 3H), 1.67-1.60; (m, 2H), 1.42-1.27; (m, 2H), 0.80; (s, 3H).

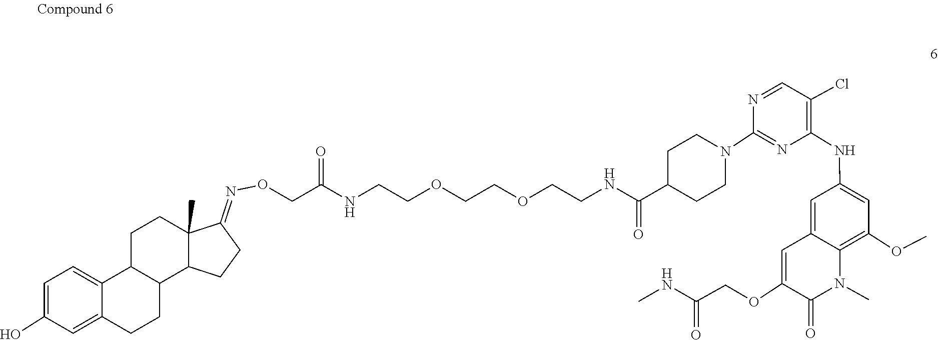 [SMILES: CNC(=O)COc1cc2cc(Nc3nc(N4CCC(C(=O)NCCOCCOCCNC(=O)CO/N=C5\CCC6C7CCc8cc(O)ccc8C7CC[C@]56C)CC4)ncc3Cl)cc(OC)c2n(C)c1=O]

Compound 6 was prepared according to procedure of scheme 2.

MS obsd. [(M+H)+] 987.1: ¹H NMR (500 MHz, DMSO) δ 8.93; (s, 1H), 8.80; (s, 1H), 8.00; (s, 1H), 7.89; (d, J=5.3 Hz, 2H), 7.78; (t, J=5.6 Hz, 2H), 7.46; (s, 2H), 7.34; (q, J=9.6 Hz, 2H), 7.00-6.91; (m, 3H), 6.42; (d, J=8.3 Hz, 2H), 6.37; (d, J=7.7 Hz, 2H), 4.45; (d, J=29.5 Hz, 5H), 4.26; (s, 2H), 3.79; (d, J=6.7 Hz, 7H), 3.33; (dt, J=16.2, 5.8 Hz, 11H), 3.19; (q, J=5.9 Hz, 5H), 3.11; (q, J=6.0 Hz, 4H), 2.83; (t, J=12.4 Hz, 4H), 2.73-2.61; (m, 4H), 2.58; (d, J=4.7 Hz, 4H), 2.48; (d, J=9.6 Hz, 4H), 2.38-2.29; (m, 3H), 2.20; (d, J=13.2 Hz, 2H), 2.06; (t, J=11.8 Hz, 2H), 1.82-1.73; (m, 4H), 1.63-1.24; (m, 15H), 1.24-1.15; (m, 4H), 0.79; (s, 3H).

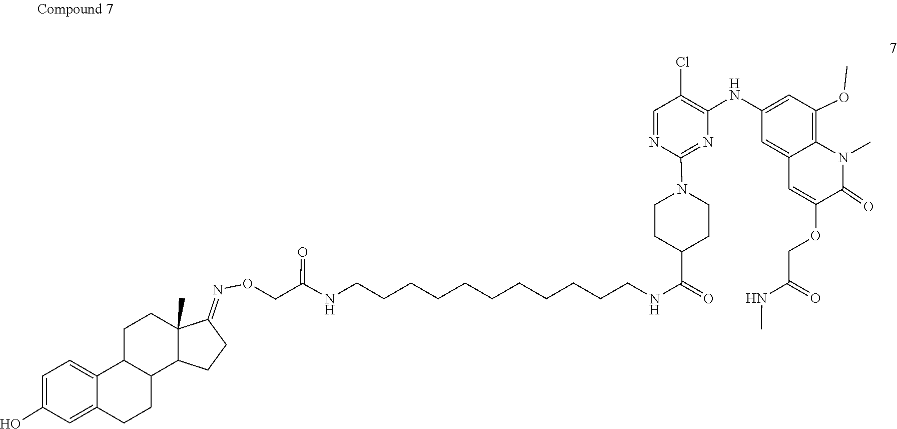 [SMILES: CNC(=O)COc1cc2cc(Nc3nc(N4CCC(C(=O)NCCCCCCCCCCCNC(=O)CO/N=C5\CCC6C7CCc8cc(O)ccc8C7CC[C@]56C)CC4)ncc3Cl)cc(OC)c2n(C)c1=O]

Compound 7 was prepared according to procedure of scheme 2.

MS obsd. [(M+H)+]: 1025.3.1H NMR (500 MHz, DMSO) δ 8.95; (br, 2H), 8.76; (s, 1H), 7.99; (s, 1H), 7.92; (s, 1H), 7.68; (t, J=5.8 Hz, 1H), 7.47; (d, J=2.1; Hz, 1H), 7.30; (t, J=5.9 Hz, 1H), 6.98-6.91; (m, 1H), 6.43; (dd, J=8.5, 2.6 Hz, 1H), 6.37; (d, J=2.6 Hz, 1H), 4.49-4.41; (m, 2H), 4.23; (s, 1H), 3.79; (d, J=5.2 Hz, 3H), 3.09-2.95; (m, 4H), 2.92; (d, J=4.2 Hz, 3H), 2.86-2.78; (m, 5H), 2.06; (s, 1H), 1.78; (t, J=14.9 Hz, 3H), 1.62-1.42; (m, 7H), 1.14; (d, J=7.5 Hz, 7H), 0.80; (s, 3H).

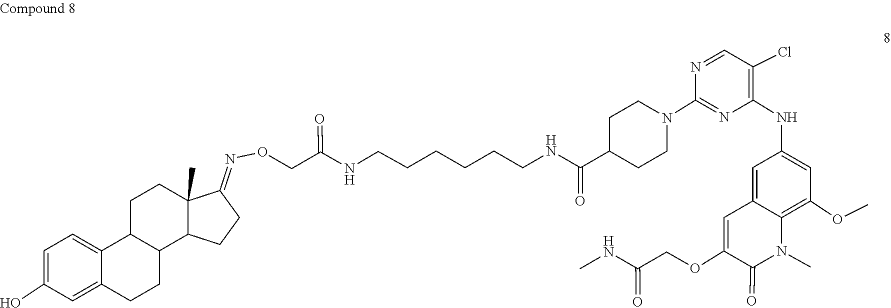 [SMILES: CNC(=O)COc1cc2cc(Nc3nc(N4CCC(C(=O)NCCCCCCNC(=O)CO/N=C5\CCC6C7CCc8cc(O)ccc8C7CC[C@]56C)CC4)ncc3Cl)cc(OC)c2n(C)c1=O]

Compound 8 was prepared according to procedure of scheme 2.

MS obsd. [(M+H)+]: 955.1. ¹H NMR (500 MHz, DMSO) δ 8.93; (s, 1H), 8.79; (s, 1H), 8.00; (s, 1H), 7.92; (d, J=5.0 Hz, 1H), 7.69; (t, J=5.6 Hz, 2H), 7.46; (q, J=2.3 Hz, 2H), 7.33; (t, J=6.0 Hz, 2H), 6.95; (d, J=10.7 Hz, 3H), 6.42; (dd, J=8.4, 2.6 Hz, 2H), 6.36; (d, J=2.7 Hz, 2H), 4.47; (s, 2H), 4.44; (d, J=12.6 Hz, 2H), 4.23; (s, 2H), 3.79; (d, J=5.4 Hz, 6H), 3.03; (p, J=6.8 Hz, 4H), 2.93; (q, J=6.5 Hz, 3H), 2.85-2.76; (m, 4H), 2.66; (dd, J=11.1, 6.1 Hz, 3H), 2.64-2.56; (m, 5H), 2.50-2.45; (m, 3H), 2.33-2.24; (m, 3H), 2.23-2.16; (m, 2H), 2.05; (t, J=11.2 Hz, 2H), 1.81-1.70; (m, 5H), 1.65-1.58; (m, 3H), 1.47-1.35; (m, 5H), 1.35-1.13; (m, 17H), 0.79; (s, 3H).

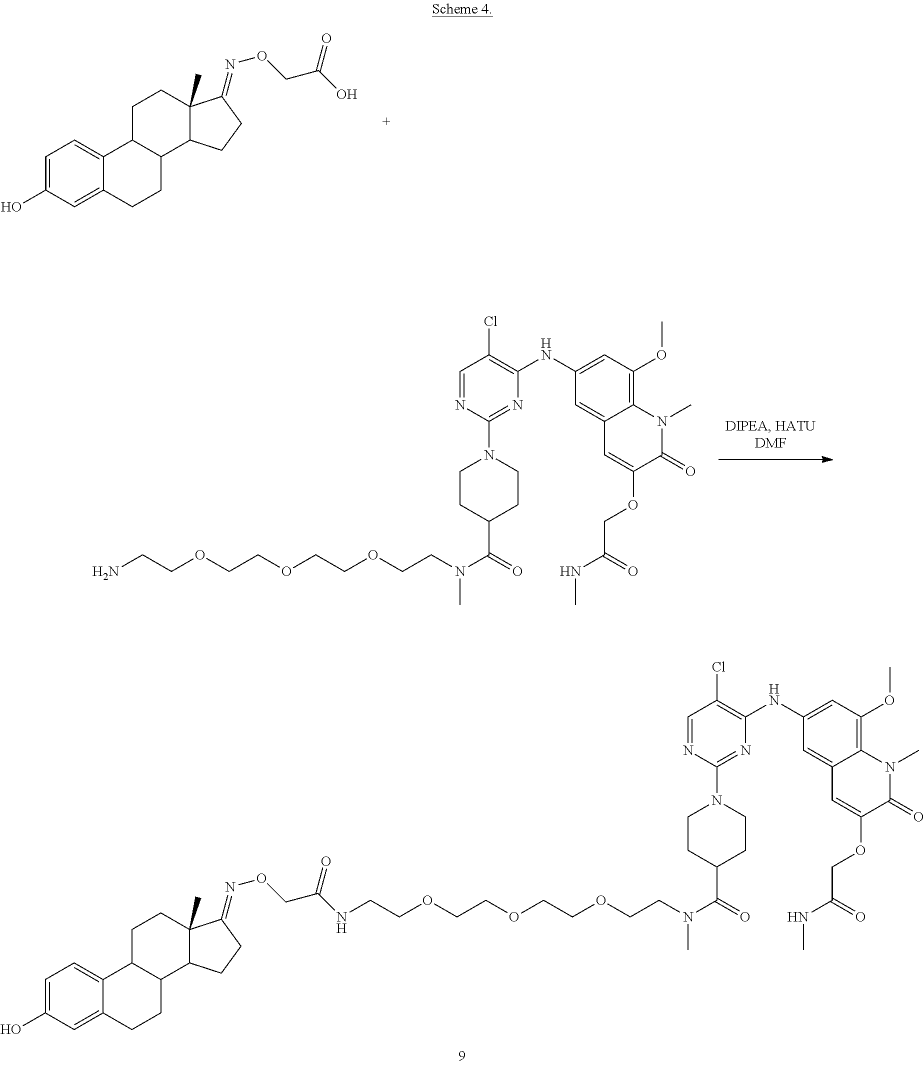 [SMILES: C[C@]12CCC3c4ccc(O)cc4CCC3C1CC/C2=N\OCC(=O)O ; CNC(=O)COc1cc2cc(Nc3nc(N4CCC(C(=O)N(C)CCOCCOCCOCCN)CC4)ncc3Cl)cc(OC)c2n(C)c1=O ; CNC(=O)COc1cc2cc(Nc3nc(N4CCC(C(=O)N(C)CCOCCOCCOCCNC(=O)CO/N=C5\CCC6C7CCc8cc(O)ccc8C7CC[C@]56C)CC4)ncc3Cl)cc(OC)c2n(C)c1=O]

A solution of 2-((((13S,E)-3-hydroxy-13-methyl-6,7,8,9,11,12,13,14,15,16-decahydro-17H-cyclopenta[a]phenanthren-17-ylidene)amino)oxy)acetic acid (15 mg, 0.06 mmol, according to lit.1 N-(2-(2-(2-(2-aminoethoxy)ethoxy)ethoxy)ethyl)-1-(5-chloro-4-((8-methoxy-1-methyl-3-(2-(methylamino)-2-oxoethoxy)-2-oxo-1,2- dihydroquinolin-6-yl)amino)pyrimidin-2-yl)-N-methylpiperidine-4-carboxamide (15 mg, 0.02 mmol, according to scheme 1), HATU (20 mg, 0.05 mmol) and DIPEA (50 uL, 0.3 mmol) in DMF (0.25 mL) was stirred at room temperature for 1 h. The crude reaction was purified by HPLC to yield 9 (2 mg, 15%) as white solid. MS obsd. [(M+H)+]: 1045.2. ¹H NMR (500 MHz, DMSO) δ 8.94; (s, 2H), 8.76; (s, 1H), 7.99; (d, J=4.6 Hz, 1H), 7.89; (d, J=6.2 Hz, 1H), 7.50-7.43; (m, 2H), 7.31; (q, J=6.3 Hz, 1H), 7.00-6.91; (m, 3H), 6.43; (td, J=8.2, 2.5 Hz, 2H), 6.37; (dd, J=8.5, 2.5 Hz, 2H), 4.55; (s, 1H), 4.47; (d, J=1.8 Hz, 2H), 4.26; (d, J=2.2 Hz, 2H), 3.79; (dd, J=5.7, 2.1 Hz, 5H), 3.47; (s, 2H), 3.46-3.36; (m, 8H), 3.35; (s, 11H), 3.23-3.14; (m, 1H), 2.99; (d, J=17.6 Hz, 2H), 2.93-2.79; (m, 4H), 2.74; (d, J=3.1 Hz, 2H), 2-2.61; (m, 1H), 2.65; (s, 3H), 2.58; (d, J=4.7 Hz, 3H), 2.38; (dd, J=18.3, 9.5 Hz, 1H), 2.21; (t, J=13.5 Hz, 2H), 2.08; (d, J=11.9 Hz, 2H), 1.80; (td, J=11.2, 3.4 Hz, 2H), 1.76; (s, 4H), 1.59; (d, J=10.5 Hz, 2H), 1.45-1.37; (m, 4H), 1.35-1.27; (m, 3H), 1.27; (s, 9H), 1.25; (d, J=12.0 Hz, 1H), 1.19; (dd, J=13.2, 6.2 Hz, 2H), 0.80; (s, 3H).

### II. CIP Compounds for Regulating Expression of the Rate-Limiting Enzyme TPH2 for Serotonin Synthesis

Methods for regulating cellular processes within distinct populations of neurons are needed to elucidate relationships between molecular mechanisms, circuits, and behavior; and to develop cell type- or circuit-selective treatments for neurological disorders. We provide here a novel, non-genetic, small molecule-based method—transcription factor-chemically induced proximity (TF-CIP)—that harnesses the cell type- and circuit-specificity of endogenous transcription factors to regulate gene expression in subsets of neurons. TF-CIP utilizes a bifunctional small molecule to draw together an “anchor” transcription factor, which naturally binds to a target gene, and a “hijacked” transcription factor, which enhances or represses transcription of the target gene. Cell type specificity is determined by the intersection of expression for each transcription factor. The TF-CIP approach can be adapted for any organism and because transcription factors are well conserved, it is reasonable to expect that the same TF-CIP small molecule can be used to modulate neuronal processes across animal species. We use a TF-CIP to modulate the expression of the rate-limiting enzyme for serotonin synthesis, TPH2, as a means to tune serotonin neurotransmission from subsets of serotonergic neurons (FIG. 29, Panel A). Although the population of serotonergic neurons is relatively small, these neurons send projections throughout the brain and serve important roles in regulating mood, anxiety, and social behavior (FIG. 29, Panel A). Achieving circuit-level specificity for a serotonin-modulatory small molecule is an improvement over current therapies, which often have undesirable side-effects due to indiscriminate targeting of serotonin signaling in the central and peripheral nervous systems. TPH2 expression is regulated by stress, sex hormones, and several transcription factors. We leverage this knowledge along with recently published single-cell RNA-sequencing and projection mapping data for serotonergic neurons as a resource for candidate TF-CIP transcription factors. Compared to genetically encoded tools for circuit-specific neuromodulation, the small molecule TF-CIP method can be adapted relatively easily for cell type and circuit-specific neuromodulation in humans.

As an example, we detail the construction and testing of TF-CIPs in cells and animals to modulate the expression of TPH2, the rate limiting enzyme for brain serotonin synthesis. Briefly, the approach involves using the steps illustrated in FIGS. 7 and 9 to choose a transcription factor (FEV) expressed only in serotonin producing neurons for the anchoring transcription factor in the TPH2 promoter. We then select ligands shown in FIG. 17 for FEV using the methods described in FIG. 8. The molecules selected from the virtual screen have significant binding to the FEV protein and in some cases to other ETS proteins by measurements using surface plasmon resonance (SPR). These are in turn chemically attached to the linkers as described above and illustrated in FIGS. 24A and 24B and in turn for ligands of transcriptional activators or repressors which could include the nBAF complex largely defined by the post mitotic neuron-specific BAF53b (ACTL6B) as well as circuit-selective transcription factors like PAX5, BRD4 and others. Because the loss of FEV results in a highly selective loss of expression of TPH2 (PMID: 12546819) and because FEV is restricted to central serotonin producing neurons (PMID: 10575032) the resultant TF-CIPs are highly selective for serotonin production in the brain. Further details may be found in Appendix A of priority provisional application serial no. 63/110,575 filed on Nov. 6, 2020, the disclosure of which is herein incorporated by reference.

### III. CIP Compounds for Enhancing Expression of Haploinsufficient Gene ARID1B

ARID1B (BAF250b) mutations are the most common cause of de novo intellectual disability and a frequent cause of autism spectrum disorder (PMID: 30349098). ARID1B is dosage-sensitive and loss of function mutations in one allele can cause intellectual disability, autism spectrum disorder, and/or Coffin-Siris syndrome. Hence, increasing ARID1B expression by 2-fold would be therapeutic. We have shown that we can do this in human induced pluripotent stem cells (iPS) taken from Coffin-Siris patients with a loss of function mutation in one allele and another normal allele. IPS cells were differentiated into neural progenitors by recruiting a transcriptional activator to the promoter of the ARID1B gene resulting in normal expression of ARID1 B (FIG. 26). This approach demonstrates that there is no barrier to the expression of ARID1B in affected human neurons and that bringing a transcription factor to its promoter will cure or mitigate intellectual disability or autism in individuals with ARID1Bhaploinsufficiency disorders.

To develop a TF-CIP to increase ARID1B expression by two-fold we found that ARID1B is controlled by another autism transcription factor, TBR1, which binds to the ARID1B regulatory regions and controls transcription of this gene (PMID: 27325115). Using the steps provided in FIG. 8 we develop ligands for TBR1. TBR1 is a highly neuron-specific gene and will give selectivity for neurons and thereby avoid off target side effects and enhance the therapeutic window. Ligands for TBR1 are attached to a linker, such as described above. These are in turn attached to a known ligand for a transcriptional activator such as BRD4, nBAF, PAXS or others using chemical linkages familiar those skilled in the art.

### IV. Methodology for Designing TF-CIPs with Cell-Specific Activity

Designing small molecule pharmaceutics with cell-specific activity is a central challenge in medicine. The relative lack of cell-specific medicines is one reason why many therapeutic drugs have unwanted and sometimes life-threatening side-effects. A special feature of CIP molecules that can engender them with cell specific-activity is that they preferentially bind to both target proteins at the same time. For example, the CIP molecule rapamycin binds with 20,000 times higher affinity to both of its target proteins, FRB and FKBP than to FRB alone (PMID: 15796538). Because of this, CIP molecules are unlikely to function in cells that only express one of the target proteins. Thus, CIP molecules intrinsically encode cell specificity from the intersection of expression of their two target proteins.

In this example, we describe a method to identify transcription factor targets for cell-specific CIPs using single-cell gene expression data and statistical overlap and correlation analyses (see FIG. 7A). First, the tissue of interest is dissected and dissociated to single cells. RNA from single cells is extracted, converted to cDNA, barcoded and sequenced on a next-generation sequencer. Following sequencing, the sequences are mapped to the genome and quantified. Individual cells are clustered according to the relatedness of their gene expression profiles. In some embodiments, the desired cell type will be indicated by the expression of endogenous cell-specific marker genes (e.g., FEV can be used as a marker for serotonergic neurons), while in others, transgenetic markers like Cre recombinase, fluorescent proteins or viral gene expression (e.g., as with retrograde labeling of projection-specific neurons) may be used to indicate a target cell cluster. In other embodiments, target cells may be purified (e.g., by fluorescence-activated cell sorting) prior to single-cell RNA-sequencing.

To identify co-expressed transcription factors in the target cells, two orthogonal approaches are used: (1) Pearson correlation and (2) statistical overlap analysis. A Pearson correlation matrix is generated representing the expression relationship between pairs of transcription factors across individual target cells. Transcription factor pairs that show positive (r>0.5) and significant (P<0.05) correlation are included in a list called Coexpressed_Targe_TFs. To perform overlap analysis, lists of cells that express a given transcription factor are generated and then analyzed with the geneOverlap package in R (Shen L, Sinai ISoMaM (2021). GeneOverlap: Test and visualize gene overlaps. R package version 1.30.0, http://shenlab-sinai.github.io/shenlab-sinai/) to generate matrices depicting the Odds Ratio (measures strength of overlap) and Jaccard Index (measures similarity between two lists) for every transcription factor pair. Transcription factor pairs that show significant (P<0.05) overlap in their expression are also added to the Target_TFs list.

Next, transcription factors pairs that show co-expression in non-target cells are excluded. For this analysis, single-cell RNA expression data from across human tissues is used (e.g., publicly available Single-Cell Atlas from the Human Protein Atlas). If applicable, single cell expression data from the target cell type is excluded from this analysis. From this subdataset of non-target cells, single-cell expression data for each of the transcription factors that are expressed in the target cells are extracted and analyzed by Pearson correlation and overlap analysis, as above. Transcription factor pairs that are not significantly correlated and which do not overlap in their expression in non-target cells are added to a list Nontarget_TFs. The lists of Target_TFs and Nontarget_TFs are intersected, resulting in a reduced list of cell-specific transcription factor pairs. Additional parameters by which transcription factor pairs may be excluded are: a) expression level of the transcription factors below a set threshold, b) the percent of target cells expressing the transcription factor pair, and c) whether a binding motif for either transcription factor is present in regulatory regions near the target gene. Following this method, we identified 14 pairs of transcription factors that show selective co-expression in dopaminergic neurons of the human substantia nigra: RORA:SOX6, RORA:AEBP2, AEBP:LCORL, PBX1:SETBP1, PBX1:PIAS1, PBX1:ZEB1, FOXP2:ZEB1, FOXP2:KLF12, MYT1L:ZNF91, ZFHX3:ZNF91, ZFHX3:ZNF420, ZNF91:ZNF420, AFF3:THRB, and TAX1BP1:TCF25. These transcription factors may serve as targets for cell-specific CIPs to enhance dopamine synthesis in patients with early-stage Parkinson's disease, as described in an earlier embodiment.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A transcription factor-chemical inducer of proximity (TF-CIP) molecule of formula I:
A-linker-B (I),
wherein:
(a) A is a first ligand that specifically binds to an anchor transcription factor (ATF) in the cell which regulates expression of a target gene in the cell;
(b) B is a second ligand that specifically binds to a transcription modulating factor (TMF) in the cell;
(c) each of the ATF and the TMF is an endogenous molecule; and
(d) the TF-CIP molecule associates the ATF and the TMF in spatial proximity, such that (i) the TMF is rewired; and (ii) the expression of the target gene that is otherwise regulated by the ATF becomes modulatable by the TMF in the cell.

2. The TF-CIP molecule of claim 1, wherein the first ligand is an inhibitor of the ATF.

3. The TF-CIP molecule of claim 1, wherein the target gene is a proapoptotic gene.

4. The TF-CIP molecule of claim 1, wherein the first ligand and the second ligand do not dissociate from each other under cellular conditions.

5. The TF-CIP molecule of claim 1, wherein the TMF is a transcription factor.

6. The TF-CIP molecule of claim 1, wherein the ATF is a transcriptional repressor and the TMF is a transcriptional activator.

7. The TF-CIP molecule of claim 1, wherein activity of the TF-CIP molecule is cell specific.

8. The TF-CIP molecule of claim 1, wherein the target gene is an oncogene.

9. The TF-CIP molecule of claim 1, wherein the target gene is a therapeutically beneficial gene. 30 10. The TF-CIP molecule of claim 1, wherein the TF-CIP molecule has a molecular weight of less than about 2,500 grams per mole (g/mole).

11. The TF-CIP molecule of claim 1, wherein each of the ATF and the TMF modulates a different pathway in the cell.

12. A method of modulating expression of a target gene in a cell, the method comprising:
contacting the cell with a chemical inducer of proximity (CIP) molecule of formula I:
A-linker-B (I),
wherein:
(a) A is a first ligand that specifically binds to an anchor transcription factor (ATF) in the cell, wherein the ATF regulates expression of the target gene in the cell;
(b) B is a second ligand that specifically binds to a transcription modulating factor (TMF) in the cell; and
(c) each of the ATF and the TMF is an endogenous molecule, and
wherein, upon the contacting, the CIP molecule associates the ATF and the TMF in spatial proximity, such that (i) the TMF is rewired; and (ii) the expression of the target gene that is otherwise regulated by the ATF is modulatable by the TMF in the cell.

13. The method of claim 12, wherein the first ligand is an inhibitor of the ATF.

14. The method of claim 12, wherein the target gene is a proapoptotic gene.

15. The method of claim 12, wherein the TMF is a transcription factor.

16. The method of claim 12, wherein the ATF is a transcriptional repressor and the TMF is a transcriptional activator.

17. The method of claim 12, wherein activity of the CIP molecule is cell specific.

18. The method of claim 12, wherein the target gene is an oncogene.

19. The method of claim 12, wherein the target gene is a therapeutically beneficial gene.

20. The method of claim 12, wherein the contacting comprises administering a therapeutically effective amount of the CIP molecule to a subject comprising the cell.

21. The method of claim 12, wherein the method results in increased or decreased expression levels of the target gene by at least about 1.5-fold, as compared to a baseline expression level of the target gene in the absence of the CIP molecule.

22. The method of claim 12, wherein each of the ATF and the TMF modulates a different pathway in the cell.

23. A method of selectively inducing death of a cancer cell that expresses a cancer driver molecule, the method comprising:
contacting the cancer cell with a chemical inducer of proximity (CIP) molecule comprising a first moiety that is linked to a second moiety via a chemical linker,
wherein:
(a) the first moiety specifically binds to the cancer driver molecule; and
(b) the second moiety specifically binds to a regulator of a proapoptotic gene in the cancer cell, and
wherein, upon the contacting, the cancer driver molecule and the regulator of the proapoptotic gene are associated in spatial proximity to form a new complex, wherein the new complex is sufficient to selectively induce death of the cancer cell.

24. The method of claim 23, wherein the new complex enhances expression of the proapoptotic gene.

25. The method of claim 23, wherein the cancer driver molecule and the regulator of the proapoptotic gene are both endogenously expressed in the cancer cell.

26. The method of claim 23, wherein the cancer driver molecule has transcriptional activating capacity.

27. The method of claim 26, wherein the first moiety specifically binds to the cancer driver molecule without negatively impacting the transcriptional activating capacity of the cancer driver molecule.

28. The method of claim 23, wherein the regulator of the proapoptotic gene is a transcriptional repressor that binds to a promoter of the proapoptotic gene and represses expression of the proapoptotic gene.

29. The method of claim 23, wherein the second moiety is an inhibitor of the regulator of the proapoptotic gene.

30. The method of claim 23, wherein the CIP molecule is more effective in inducing the death of the cancer cell as compared to a control molecule lacking one of the first moiety and the second moiety.

31. The method of claim 23, wherein the CIP molecule is more effective in inducing the death of the cancer cell as compared to inducing death of a control cell, wherein the cancer cell exhibits a higher expression of the cancer driver molecule than the control cell.

32. The method of claim 23, wherein the contacting comprises administering a therapeutically effective amount of the CIP molecule to a subject comprising the cancer cell.

33. A method of treating cancer in a subject in need thereof, the method comprising:
administering to the subject an effective amount of a chemical inducer of proximity (CIP) molecule comprising a first moiety that is linked to a second moiety via a chemical linker,
wherein:
(a) the first moiety specifically binds to an endogenous molecule selectively expressed in a cancer cell; and
(b) the second moiety specifically binds to an endogenous regulator of a proapoptotic gene in the cancer cell, and
wherein, upon the administering, the endogenous molecule selectively expressed in the cancer cell and the endogenous regulator of the proapoptotic gene are associated in spatial proximity to form a new complex in the cancer cell of the subject, wherein the new complex is sufficient to selectively induce death of the cancer cell, thereby treating cancer in said subject.

34. The method of claim 33, wherein the new complex enhances expression of the proapoptotic gene.

35. The method of claim 33, wherein both the endogenous molecule selectively expressed in the cancer cell and the endogenous regulator of the proapoptotic gene are proteins.

36. The method of claim 33, wherein the endogenous molecule selectively expressed in the cancer cell has transcriptional activating capacity.

37. The method of claim 36, wherein the first moiety specifically binds to the endogenous molecule selectively expressed in the cancer cell without negatively impacting the transcriptional activating capacity of the endogenous molecule.

38. The method of claim 33, wherein the endogenous regulator of the proapoptotic gene is a protein.

39. The method of claim 33, wherein the endogenous molecule selectively expressed in the cancer cell is an oncogenic transcription factor.

40. The method of claim 33, wherein the regulator of the proapoptotic gene is a transcriptional repressor that binds to a promoter of the proapoptotic gene and represses expression of the proapoptotic gene.

41. The method of claim 33, wherein the second moiety is an inhibitor of the regulator of the proapoptotic gene.

42. The method of claim 33, wherein the CIP molecule is more effective in inducing the death of the cancer cell as compared to a control molecule lacking one of the first moiety and the second moiety.

43. The method of claim 33, wherein the CIP molecule is more effective in inducing the death of the cancer cell as compared to inducing death of a control cell, wherein the cancer cell exhibits a higher level of expression of the endogenous molecule selectively expressed in the cancer cell or the endogenous regulator of the proapoptotic gene than that of the control cell.


SEQUENCE LISTING (14 sequences)

SEQ ID NO: 1 - DNA, 289 bp, synthetic construct
  source 1..289: mol_type = other DNA
tacggccctcttgctttcgaggaatgtcggtgcaccctacgggcgtcctgctttctaggaacttcggtcctccttacgcgcctcctgctgtcgaggaatctcggtccactaccggcctctcgctttctaggaatttcggtccagcacgggcctcttgctgtcgaggaatttaggtccacgctaggggcctcttgctttctaggaaattcggaccaccttcgggcccctcgctttcgaggaatatcggtcctcctacgagcctcatgctttccaggaatatcggtcgacc

SEQ ID NO: 2 - DNA, 288 bp, synthetic construct
  source 1..288: mol_type = other DNA
aatgactcatctgctttctaggaatttcctgggcaagccatgtgatttgctttcgaggaatgcttttgtgcagttcagtctcctgctttctaggaatggagctgtcccttctcccaaaaagctttctaggaatgtaacttgtaagggctctgagctgctctctaggaattctggggaatcagccctgggttggctttctaggaattttgggggaaccagtaatttgtggctttctaggaatttttccatttccattcagcctgtgctttcgaggaattttatagtcct

SEQ ID NO: 3 - DNA, 212 bp, synthetic construct
  source 1..212: mol_type = other DNA
ttgcaagtgactgttttctaggtactatgactagaggtcagtataaatgctttccaataaagcatgtccagccgttttttcttttcctaaaaggatttttttttctcagctcccgccgtcctggacagccagtcacctggggtggaccgaaatcccgcgacagcaagaggcccgtacctctccggactgctttccagggagtagaagacaga

SEQ ID NO: 4 - DNA, 185 bp, synthetic construct
  source 1..185: mol_type = other DNA
taaaaaaattatttttccaagaagaaaataaaccctgtcttctactccctggaaagcagtccggagaggacttttttctaattcatagaaattatttgttgattcatacatgcaaattcttagaattcctggcatgcagagtaaattcattgcaagtgactgttttctaggtactatgactagag

SEQ ID NO: 5 - DNA, 206 bp, synthetic construct
  source 1..206: mol_type = other DNA
cctcctaagtatttctccaagaaacacgaaaacagagccgggggctgcctggaaagatcgctccaggagttaaaatgtcattttttaggaaggctttccgtaaacctgagaacttcttgaaaggctgtaagtggtaaaaacagtccagttttctaggagagggatgcccctacgggcctcttgctgtcgcgggatttcggtccacc

SEQ ID NO: 6 - DNA, 136 bp, synthetic construct
  source 1..136: mol_type = other DNA
aggtttctgttgcagtctaggaaaaatggagaagggtagctttttttcctgaaaataaaaagaaaaatcgttttttcttttcctaaaaggatttttttttctccaggtgactggctgtccaggacggcgggagctg

SEQ ID NO: 7 - DNA, 172 bp, synthetic construct
  source 1..172: mol_type = other DNA
taccacttacagcctttcaagaagttctcaggtttacgggcctcttgctgtcgcgggatttcggtccacctacggaaagccttcctaaaaaatgacattttaactcctggagcgatctttccaggcagcccccggctctgttttcgtgtttcttggagaaatacttaggagg

SEQ ID NO: 8 - DNA, 54 bp, synthetic construct
  source 1..54: mol_type = other DNA
ctccggtaaacacgccagtgtttgtaaacaaacccgcttgggtaaacaagccca

SEQ ID NO: 9 - DNA, 84 bp, synthetic construct
  source 1..84: mol_type = other DNA
agttactccggtaaacacgccagggacgcccattgtttgtaaacaaacccgccagaagagacttgggtaaacaagcccagaaaa

SEQ ID NO: 10 - DNA, 112 bp, synthetic construct
  source 1..112: mol_type = other DNA
agttactccggtaaacacgccagggacgcccattgtttgtaaacaaacccgccagatctggcgggtttgtttacaaacaatgggagagacttgggtaaacaagcccagaaaa

SEQ ID NO: 11 - DNA, 140 bp, synthetic construct
  source 1..140: mol_type = other DNA
agttactccggtaaacacgccagggacgcgtccctggcgtgtttaccggagtaactcccattgtttgtaaacaaacccgccagatctggcgggtttgtttacaaacaatgggagagacttgggtaaacaagcccagaaaa

SEQ ID NO: 12 - DNA, 108 bp, synthetic construct
  source 1..108: mol_type = other DNA
ctccggtaaacacgccagctggcgtgtttaccggagtgtttgtaaacaaacccgcgggtttgtttacaaacacttgggtaaacaagcccatgggcttgtttacccaag

SEQ ID NO: 13 - DNA, 168 bp, synthetic construct
  source 1..168: mol_type = other DNA
agttactccggtaaacacgccagggacgcgtccctggcgtgtttaccggagtaactcccattgtttgtaaacaaacccgccagatctggcgggtttgtttacaaacaatgggagagacttgggtaaacaagcccagaaaattttctgggcttgtttacccaagtctct

SEQ ID NO: 14 - DNA, 224 bp, synthetic construct
  source 1..224: mol_type = other DNA
agttactccggtaaacacgccagggacgcgtccctggcgtgtttaccggagtaactcccattgtttgtaaacaaacccgccagatctggcgggtttgtttacaaacaatgggagagacttgggtaaacaagcccagaaaattttctgggcttgtttacccaagtctctcagagaggctgtaaacaagccaacgggcgcccgttggcttgtttacagcctctctg